SEQUENCE LISTING (44 sequences)

SEQ ID NO: 1 - DNA, 20 bp, Artificial Sequence
  P5 primer
aatgatacggcgaccaccga

SEQ ID NO: 2 - DNA, 24 bp, Artificial Sequence
  P7 primer
caagcagaagacggcatacgagat

SEQ ID NO: 3 - protein, 20 aa, Artificial Sequence
  FA-PA
FAGVAMPGAEDDVVGSGSGK

SEQ ID NO: 4 - protein, 20 aa, Artificial Sequence
  AFA-PA
AFAGVAMPGAEDDVVGSGSK

SEQ ID NO: 5 - protein, 19 aa, Artificial Sequence
  AA-PA
AAGVAMPGAEDDVVGSGSK

SEQ ID NO: 6 - protein, 103 aa, Agrobacterium tumefaciens
  MISC_FEATURE (1)..(103): Scaffold for binder selection 1
MSDSPVDLKPKPKVKPKLERPKLYKVMLLNDDYTPREFVTVVLKAVFRMSEDTGRRVMMTAHRFGSAVVVVCERDIAETKAKEATDLGKEAGFPLMFTTEPEE

SEQ ID NO: 7 - protein, 164 aa, Homo sapiens
  MISC_FEATURE (1)..(164): Scaffold for binder selection 2
QVSVQPNFQQDKFLGRWFSAGLASNSSWLREKKAALSMAKSVVAPATDGGLNLTSTFLRKNQCETRTMLLQPAGSLGSYSYRSPHFGSTYSVSVVETDYDQYALLYSQGSKGPGEDFRMATLYSRTQTPRAELKEKFTAFSKAQGFTEDTIVFLPQTDKCMTEQ

SEQ ID NO: 8 - protein, 5 aa, Artificial Sequence
  Bait peptide for binder selection 1
  MISC_FEATURE (1)..(1): N-terminal pyrazole methaniminated
  MISC_FEATURE (2)..(2): Xaa is any natural amino acid
  MISC_FEATURE (4)..(5): an internal nine atom polyethylene glycol spacer
  MISC_FEATURE (5)..(5): C-terminal biotinylated
FXGGK

SEQ ID NO: 9 - protein, 174 aa, Homo sapiens
  MISC_FEATURE (1)..(174): Scaffold for binder selection 3
GPVPTPPDNIQVQENFNISRIYGKWYNLAIGSTSPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQ

SEQ ID NO: 10 - protein, 5 aa, Artificial Sequence
  Bait peptide for binder selection 2
  MISC_FEATURE (1)..(1): N-terminal pyrazole methaniminated
  MISC_FEATURE (2)..(2): Xaa is any natural amino acid
  MISC_FEATURE (4)..(5): An internal nine atom polyethylene glycol spacer
  MISC_FEATURE (5)..(5): C-terminal biotinylated
LXGGK

SEQ ID NO: 11 - DNA, 46 bp, Artificial Sequence
  amRT_Cs2 oligonucleotide
  misc_feature (21)..(30): n is a, c, g, or t
ttcgtagtccgcgacactagnnnnnnnnnngttaatggactgagtg

SEQ ID NO: 12 - DNA, 46 bp, Artificial Sequence
  amRT_Cs4 oligonucleotide
  misc_feature (21)..(30): n is a, c, g, or t
ttcgtagtccgcgacactagnnnnnnnnnncagtaccgactgagtg

SEQ ID NO: 13 - DNA, 46 bp, Artificial Sequence
  amRT_Cs5 oligonucleotide
  misc_feature (21)..(30): n is a, c, g, or t
ttcgtagtccgcgacactagnnnnnnnnnngttggttaactgagtg

SEQ ID NO: 14 - DNA, 46 bp, Artificial Sequence
  amRT_Cs1 oligonucleotide
  misc_feature (21)..(30): n is a, c, g, or t
ttcgtagtccgcgacactagnnnnnnnnnnttaagtcgactgagtg

SEQ ID NO: 15 - DNA, 25 bp, Artificial Sequence
  coding tag oligonucleotide
cactcagtcagactattcactcagt

SEQ ID NO: 16 - DNA, 20 bp, Artificial Sequence
  am-PL1 oligonucleotide
catagactagtagccggaac

SEQ ID NO: 17 - DNA, 20 bp, Artificial Sequence
  am-PL2oligonucleotide
tcgacgtaatgacaccgctc

SEQ ID NO: 18 - DNA, 45 bp, Artificial Sequence
  CT_PL1_S6 adaptor molecule
  misc_feature (20)..(21): an internal six atom polyethylene glycol spacer
gttccggctactagtctatgcactcagtttttcctgtcactcagt

SEQ ID NO: 19 - DNA, 45 bp, Artificial Sequence
  CT_PL2_S6 adaptor molecule
  misc_feature (20)..(21): an internal six atom polyethylene glycol spacer
gagcggtgtcattacgtcgacactcagtttttcctgtcactcagt

SEQ ID NO: 20 - DNA, 20 bp, Artificial Sequence
  coding tag barcode sequence 1
tggtagagccacaaacagcc

SEQ ID NO: 21 - DNA, 20 bp, Artificial Sequence
  coding tag barcode sequence 2
ggtacaagcaacgatctcca

SEQ ID NO: 22 - DNA, 20 bp, Artificial Sequence
  coding tag barcode sequence 3
ggaccatctgaatcatgcgc

SEQ ID NO: 23 - DNA, 20 bp, Artificial Sequence
  coding tag barcode sequence 4
ggatgacacgaactcacgac

SEQ ID NO: 24 - DNA, 20 bp, Artificial Sequence
  coding tag barcode sequence 5
ggcgatcacagacattaacc

SEQ ID NO: 25 - DNA, 20 bp, Artificial Sequence
  coding tag barcode sequence 6
cacagccgataattgcagac

SEQ ID NO: 26 - DNA, 20 bp, Artificial Sequence
  coding tag barcode sequence 7
ggtacagacactgcgacaac

SEQ ID NO: 27 - DNA, 20 bp, Artificial Sequence
  coding tag barcode sequence 8
gtggcaattcgtcgcaatac

SEQ ID NO: 28 - DNA, 20 bp, Artificial Sequence
  coding tag barcode sequence 9
gggtcatcacggctcatcat

SEQ ID NO: 29 - DNA, 20 bp, Artificial Sequence
  coding tag barcode sequence 10
gccagatgtcaacacagcta

SEQ ID NO: 30 - DNA, 20 bp, Artificial Sequence
  coding tag barcode sequence 11
ccgccaaacaaatgtgtgca

SEQ ID NO: 31 - DNA, 20 bp, Artificial Sequence
  coding tag barcode sequence 12
atacacgctcggaagactgc

SEQ ID NO: 32 - DNA, 20 bp, Artificial Sequence
  coding tag barcode sequence 13
atgatgaccgcactgactgg

SEQ ID NO: 33 - DNA, 20 bp, Artificial Sequence
  coding tag barcode sequence 14
ggacagcagatccacctaag

SEQ ID NO: 34 - DNA, 20 bp, Artificial Sequence
  coding tag barcode sequence 15
cctgtgagagaagcagacac

SEQ ID NO: 35 - DNA, 20 bp, Artificial Sequence
  coding tag barcode sequence 16
ccgacagatcaaggcagtta

SEQ ID NO: 36 - DNA, 20 bp, Artificial Sequence
  coding tag barcode sequence 17
aatcgcagccaagtgagtga

SEQ ID NO: 37 - DNA, 20 bp, Artificial Sequence
  coding tag barcode sequence 18
atagatgacgcaccacggtc

SEQ ID NO: 38 - DNA, 20 bp, Artificial Sequence
  coding tag barcode sequence 19
agacacgacacactggctta

SEQ ID NO: 39 - DNA, 20 bp, Artificial Sequence
  coding tag barcode sequence 20
aggagacgccacatcgtatc

SEQ ID NO: 40 - DNA, 41 bp, Artificial Sequence
  adaptor molecule 1
  misc_feature (20)..(21): an internal three carbon spacer
  misc_feature (41)..(41): a 3′-terminal three-carbon spacer
ggctgtttgtggctctaccaggtaagagcgactgtagtgtg

SEQ ID NO: 41 - DNA, 41 bp, Artificial Sequence
  adaptor molecule 2
  misc_feature (20)..(21): an internal three-carbon spacer
  misc_feature (41)..(41): a 3′-terminal three-carbon spacer
tggagatcgttgcttgtaccggtaagagccgatgtagtgtg

SEQ ID NO: 42 - DNA, 41 bp, Artificial Sequence
  adaptor molecule 3
  misc_feature (20)..(21): an internal three-carbon spacer
  misc_feature (41)..(41): a 3′-terminal three-carbon spacer
ggctgtttgtggctctaccaagagatggcacgtggtaagag

SEQ ID NO: 43 - DNA, 41 bp, Artificial Sequence
  adaptor molecule 4
  misc_feature (20)..(21): an internal three-carbon spacer
  misc_feature (41)..(41): a 3′-terminal three-carbon spacer
tggagatcgttgcttgtaccagagatggtgcgtggtaagag

SEQ ID NO: 44 - protein, 52 aa, Artificial Sequence
  Capping oligonucleotide
  MISC_FEATURE (52)..(52): a 3′-terminal three-carbon spacer
GACTGGAGTTCAGACGTGTGCTCTTCCGATCTTTCTACTCTTCTCACTCAGT

(19) United States
(12) Patent Application Publication
(10) Pub. No.: US 2023/0056532 A1
(43) Pub. Date: Feb. 23, 2023

(54) METHODS FOR INFORMATION TRANSFER AND RELATED KITS

(71) Applicant: ENCODIA, INC., San Diego, CA (US)

(72) Inventors: Kevin L. GUNDERSON, San Diego, CA (US); Norihito MURANAKA, San Diego, CA (US)

(73) Assignee: ENCODIA, INC., San Diego, CA (US)

(21) Appl. No.: 17/790,876

(22) PCT Filed: Jan. 5, 2021
(86) PCT No.: PCT/US2021/012220

§ 371 Date: Jul. 5, 2022

Related U.S. Application Data
(60) Provisional application No. 62/958,202, filed on Jan. 7, 2020.

(51) Int. Cl. G01N 33/543; C40B 20/04; C40B 70/00; C12Q 1/6813

(52) U.S. Cl. CPC G01N 33/54353; G01N 33/54306; C40B 20/04; C40B 70/00; C12Q 1/6813

(57) ABSTRACT

The present disclosure relates to methods and kits for analyzing a macromolecule including information transfer between molecules, such as transfer of identifying information between nucleic acid molecules. In some embodiments, the macromolecule for analysis comprises a peptide, a polypeptide, or a protein. In some embodiments, the present disclosure relates to macromolecule analysis methods which employ barcoding and nucleic acid encoding of molecular recognition events, and/or detectable labels. Provided herein is a programmable system for information transfer comprising one or more adaptor molecules.

### RELATED APPLICATION

The present application claims priority to U.S. provisional patent application No. 62/958,202, filed on Jan. 7, 2020, the disclosure and content of which are incorporated herein by reference in their entireties for all purposes.

### SEQUENCE LISTING ON ASCII TEXT

This patent application file contains a Sequence Listing submitted in computer readable ASCII text format (file name: 4614-2002040_SeqList.20210105_ST25.txt, recorded: 5 Jan. 2021, size: 15,298 bytes). The content of the Sequence Listing file is incorporated herein by reference in its entirety.

### TECHNICAL FIELD

The present disclosure relates to methods and kits for analyzing a macromolecule including information transfer between molecules, such as transfer of identifying information between nucleic acid molecules. In some embodiments, the macromolecule for analysis comprises a peptide, a polypeptide, or a protein. In some embodiments, the present disclosure relates to macromolecule analysis methods which employ barcoding and nucleic acid encoding of molecular recognition events, and/or detectable labels. Also provided herein is a programmable system for information transfer comprising, using or involving one or more adaptor molecules.

### BACKGROUND

Highly-parallel characterization and recognition of macromolecules such as proteins remains a challenge. Affinity-based assays may be used for this purpose but is often difficult due to several key challenges. One significant challenge is multiplexing the readout of a collection of affinity agents to a collection of cognate macromolecules, for example using affinity agents with detectable labels; another challenge is minimizing cross-reactivity between the affinity agents and off-target macromolecules; a third challenge is developing an efficient high-throughput read out platform. An example of this problem occurs in proteomics in which one goal is to identify and quantitate numerous proteins in a sample. Currently this is a formidable task to accomplish in a high-throughput way. Assays such as immunoassays and mass spectrometry based methods have been used but are limited at both the sample and analyte level, limited sensitivity and dynamic range, and cross-reactivity and background signals. Ligation-detection-reaction (LDR) based microarrays can be used for detection by utilizing the specificity of the ligase enzyme to covalently join two adjacently hybridized probes. In LDR, a zip-oligo is attached to the 3′-end of one probe and fluorescent label to the 5′-end of the other probe (See e.g., Hultman et al., BMC Microbiol. (2008) 8: 237). Other universal DNA microarray methods which use nucleic acid information transfer including polymerase chain reaction/ligase detection reaction (PCR/LDR) with “zip-code” hybridization have also been used to detect low abundance mutations (See e.g., Ladner et al., Lab Invest. (2001) 81(8):1079-86); Gerry et al., J Mol Biol (1999) 292:251-262).

However, there remains a need for improved techniques relating to macromolecule analysis, with applications to protein sequencing and/or analysis, as well as to products, methods and kits for accomplishing the same. There is a need for proteomics technology for performing macromolecule analysis that is flexible, highly-parallelized, accurate, sensitive, and high-throughput. The present disclosure fulfills these and other related needs.

These and other aspects of the invention will be apparent upon reference to the following detailed description. To this end, various references are set forth herein which describe in more detail certain background information, procedures, compounds and/or compositions, and are each hereby incorporated by reference in their entireties.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

Provided herein is method for analyzing a macromolecule comprising the steps of: (a) providing a macromolecule and an associated recording tag joined to a support; (b) contacting the macromolecule with a binding agent capable of binding to the macromolecule, wherein the binding agent comprises a coding tag with identifying information regarding the binding agent, to allow binding between the macromolecule and the binding agent; (c) providing an adaptor molecule comprising a first hybridization sequence and a secondary tag, wherein the first hybridization sequence is substantially complementary to at least a portion of the coding tag, to allow hybridization between the first hybridization sequence and the coding tag, wherein step (c) is performed before, after or simultaneously with step (b); (d) transferring information of the secondary tag to the recording tag to generate an extended recording tag, wherein the information of the secondary tag is transferred from the adaptor molecule to the recording tag after the coding tag associated with the binding agent hybridizes with the first hybridization sequence on the adaptor molecule; and (e) analyzing the extended recording tag.

In some embodiments, step (b) of the method comprises contacting a plurality of macromolecules with a plurality of binding agents and step (c) comprises providing a plurality of adaptor molecules, wherein the plurality of adaptor molecules comprises at least one adaptor molecule capable of hybridizing to at least one coding tag associated with the binding agent. In some embodiments, multiple coding tags associated with the binding agent are configured to hybridize to adaptor molecules comprising the same secondary tag. In some embodiments, the adaptor molecule further comprises a second hybridization sequence substantially complementary to a sequence at the 3′ terminus of the recording tag or substantially complementary to a region on the recording tag generated from a previous information transfer of the secondary tag from the adaptor molecule to the recording tag, and wherein information transfer of the secondary tag from the adaptor molecule to the recording tag occurs after: the first hybridization sequence on the adaptor molecule hybridizes to the coding tag of the binding agent; and the second hybridization sequence of the adaptor molecule hybridizes to a portion of the recording tag.

In some embodiments, the macromolecule is a polypeptide, analyzing the macromolecule comprises determining at least a portion of an amino acid sequence of the polypeptide, and recording tag, the coding tag and the adaptor molecule comprise a DNA molecule, an RNA molecule, a PNA molecule, a BNA molecule, an XNA, molecule, an LNA molecule, a γPNA molecule, or a combination thereof.

In some embodiments, the second hybridization sequence on the adaptor molecule comprises the secondary tag information or portion thereof on a different adaptor molecule.

In some embodiments, information transfer from the secondary tag to the recording tag is mediated by a DNA ligase or DNA polymerase.

In some embodiments, information transfer from the secondary tag to the recording tag is mediated by chemical ligation.

In some embodiments, the adaptor molecule comprises a spacer to stop extension after transfer of information from the secondary tag to the recording tag.

In some embodiments, the secondary tag comprises a binding cycle specific sequence.

In some embodiments, the adaptor molecule comprises from 5′ to 3′ direction: the first hybridization sequence, the secondary tag, and the second hybridization sequence, and the secondary tag comprises an identifying information regarding the binding agent, a binding cycle-specific barcode, a unique molecular identifier, or a combination thereof.

In some embodiments, the binding agent is configured to bind to an N-terminal amino acid (NTAA) residue of the polypeptide.

In some embodiments, the method further comprises the following step: (a′) modifying an N-terminal amino acid (NTAA) residue of the polypeptide, thereby producing a modified NTAA residue, and the binding agent is configured to bind to the modified NTAA residue of the polypeptide.

In some embodiments, the method further comprises the following steps after step (d) and before step (e): (i) removing the modified NTAA residue of the polypeptide to expose immediately adjacent amino acid residue of the polypeptide as a new NTAA residue; (ii) modifying the new NTAA residue, thereby producing a new modified NTAA residue; (iii) contacting the polypeptide with a second binding agent capable of binding to the new modified NTAA residue of the polypeptide, wherein the second binding agent comprises a second coding tag with identifying information regarding the second binding agent, to allow binding between the polypeptide and the binding agent; (iv) providing a second adaptor molecule comprising a second first hybridization sequence substantially complementary to at least a portion of the second coding tag, and a second secondary tag, to allow hybridization between the second first hybridization sequence and the second coding tag; (v) transferring an information of the second secondary tag to the extended recording tag, wherein the information of the second secondary tag is transferred from the second adaptor molecule to the extended recording tag after the second coding tag associated with the second binding agent hybridizes with the second first hybridization sequence on the second adaptor molecule.

In some embodiments, analyzing the extended recording tag comprises performing one of the following nucleic acid sequencing method: sequencing by synthesis, sequencing by ligation, sequencing by hybridization, polony sequencing, ion semiconductor sequencing, pyrosequencing, single molecule real-time sequencing, nanopore-based sequencing, or direct imaging of DNA using advanced microscopy.

In some embodiments, the binding agent is an aminopeptidase or variant, mutant, or modified protein thereof; an aminoacyl tRNA synthetase or variant, mutant, or modified protein thereof; an anticalin or variant, mutant, or modified protein thereof; a ClpS, ClpS2, or variant, mutant, or modified protein thereof; a UBR box protein or variant, mutant, or modified protein thereof; or a modified small molecule that binds amino acid(s), i.e. vancomycin or a variant, mutant, or modified molecule thereof; or an antibody or binding fragment thereof; or any combination thereof.

Provided herein is a kit for analyzing a macromolecule comprising: a binding agent comprising a coding tag, which comprises identifying information regarding the binding agent, wherein the binding agent is configured to bind the macromolecule associated with a recording tag joined to a support; an adaptor molecule comprising a first hybridization sequence substantially complementary to at least a portion of the coding tag, and a secondary tag, wherein an information of the secondary tag is configured for transfer from the adaptor molecule to the recording tag to generate an extended recording tag after the coding tag associated with the binding agent hybridizes with the first hybridization sequence on the adaptor molecule; optionally, a means for transfer the information of the secondary tag or a means for analyzing the extended recording tag. In some embodiments, the kit comprises a plurality of binding agents or a plurality of adaptor molecules, wherein the plurality of adaptor molecules comprises at least one adaptor molecule capable of hybridizing to at least one coding tag associated with the binding agent. In some embodiments, the adaptor molecule of the kit further comprises a second hybridization sequence substantially complementary to a sequence at the 3′ terminus of the recording tag or substantially complementary to a region on the recording tag generated from a previous information transfer of the secondary tag from the adaptor molecule to the recording tag, and wherein information transfer of the secondary tag from the adaptor molecule to the recording tag is configured to occur after: the first hybridization sequence on the adaptor molecule hybridizes to the coding tag of the binding agent; and the second hybridization sequence of the adaptor molecule hybridizes to a portion of the recording tag.

In some of embodiments of the kit, the macromolecule is a polypeptide, analyzing the macromolecule comprises determining at least a portion of an amino acid sequence of the polypeptide, and recording tag, the coding tag and the adaptor molecule comprise a DNA molecule, an RNA molecule, a PNA molecule, a BNA molecule, an XNA, molecule, an LNA molecule, a γPNA molecule, or a combination thereof.

In some of embodiments of the kit, the means for transfer the information of the secondary tag comprises DNA ligase or DNA polymerase, and the means for analyzing the extended recording tag comprises a sequencing primer.

In some of embodiments of the kit, the adaptor molecule comprises from 5′ to 3′ direction: the first hybridization sequence, the secondary tag, and the second hybridization sequence, and the secondary tag comprises an identifying information regarding the binding agent, a binding cycle-specific barcode, a unique molecular identifier, or a combination thereof.

In some of embodiments of the kit, the kit further comprises a support for immobilizing the polypeptide and/or the recording tag.

In some of embodiments of the kit, the binding agent is an aminopeptidase or variant, mutant, or modified protein thereof; an aminoacyl tRNA synthetase or variant, mutant, or modified protein thereof; an anticalin or variant, mutant, or modified protein thereof; a ClpS, ClpS2, or variant, mutant, or modified protein thereof; a UBR box protein or variant, mutant, or modified protein thereof; or a modified small molecule that binds amino acid(s), i.e. vancomycin or a variant, mutant, or modified molecule thereof; or an antibody or binding fragment thereof; or any combination thereof.

In some of embodiments of the kit, the adaptor molecule further comprises a second hybridization sequence substantially complementary to a portion of the recording tag.

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIG. 1A-FIG. 1D depicts an exemplary macromolecule analysis assay involving information transfer using an adaptor molecule with a first hybridization sequence and a secondary tag. In FIG. 1A, a peptide to be analyzed is joined to a recording tag immobilized on a support. In FIG. 1B, the peptide is contacted with a binding agent associated with a coding tag and the binding agent interacts with the peptide to be analyzed. In FIG. 1C, an adaptor molecule comprising a first hybridization sequence (1^{st} hyb sequence) and secondary tag is introduced. The first hybridization sequence contains a sequence complementary to the coding tag associated with the binding agent. Once the adaptor molecule is in place (via hybridization to the coding tag), information from the secondary tag on the adaptor molecule containing identifying information regarding the binding agent is transferred from the adaptor molecule to the recording tag via ligation, thereby generating an extended recording tag. After information transfer, the adaptor molecule (portion besides the secondary tag) may be released by digestion, such as by treating with USER Enzyme. After information transfer, the binding agent may be optionally removed, as shown in FIG. 1D. A cycle of steps shown in FIG. 1B-1D may be repeated one or more times to further extend the recording tag.

FIG. 2A-FIG. 2D depicts an exemplary macromolecule analysis assay involving information transfer using an adaptor molecule with a first hybridization sequence, a secondary tag, and a second hybridization sequence. In FIG. 2A, a peptide to be analyzed is joined to a recording tag immobilized on a support. In FIG. 2B, the peptide is contacted with a binding agent associated with a coding tag and the binding agent interacts with the peptide to be analyzed. In FIG. 2C, an adaptor molecule comprising a first hybridization sequence (1^{st} hyb sequence), secondary tag, and second hybridization sequence (2^{nd} hyb sequence) is introduced. The first hybridization sequence contains a sequence complementary to the coding tag associated with the binding agent. The second hybridization sequence contains a sequence complementary to a portion of the recording tag. Once the adaptor molecule is in place (via hybridization to the coding tag and recording tag), information from the secondary tag on the adaptor molecule containing identifying information regarding the binding agent is transferred from the adaptor molecule to the recording tag (e.g. via extension). The adaptor molecule contains a linker between the first hybridization sequence and the secondary tag, which is used to stop polymerase extension. After information transfer, the binding agent may be optionally removed, as shown in FIG. 2D. A cycle of steps shown in FIG. 2B-2D may be repeated one or more times to further extend the recording tag.

FIG. 3 depicts a model or exemplary assay system for information transfer using a splint adaptor molecule containing a first hybridization sequence complementary to a region on the coding tag and a second hybridization sequence complementary to a region on the recording tag.

FIG. 4 depicts an exemplary embodiment for information transfer based on hybridization and extension reactions. FIG. 4A shows a splint adaptor molecule containing a first hybridization sequence (Payload seq 1, PL1) complementary to a region on the coding tag (PL1′), followed by a PEG-based linker, a spacer sequence (Sp′), a barcode sequence (BC′) and another spacer sequence (Sp′) complementary to a region on the recording tag (Sp). FIG. 4B shows encoding yield in the information transfer assay utilizing an engineered F-binder. The encoding assay was performed with the F-binder conjugated with two different coding tags that contain oligonucleotides complementary to hybridization sequences Payload seq 1 (PL1) and Payload seq 1 (PL2); splint adaptor molecules as shown in FIG. 4A containing from 5′end to 3′ end a first hybridization sequence (either PL1 or PL2), a secondary tag (Sp′ and BC′) and a second hybridization sequence (Sp′) were added to allow hybridization between the splint adaptor molecule, the coding tag and the recording tag. Coding tags were fused to 3 peptides (AA-PA, AFA-PA or FA-PA, SEQ ID NOs: 3-5) or used without peptide. Encoding yield on each peptide was obtained by evaluating encoded recording tag corresponding to the attached peptide by a NGS readout. High encoding yield was observed only for the target peptide FA-PA having F as the N-terminal amino acid.

### DETAILED DESCRIPTION

Provided herein are methods and kits for analyzing a macromolecule. In some embodiments, the analysis employs barcoding and nucleic acid encoding of molecular recognition events, and/or detectable labels. The provided method comprises: (a) providing a macromolecule and an associated recording tag joined to a support; (b) contacting the macromolecule with a binding agent capable of binding to the macromolecule, wherein the binding agent comprises a coding tag with identifying information regarding the binding agent, to allow binding between the macromolecule and the binding agent; (c) providing an adaptor molecule comprising a first hybridization sequence substantially complementary to at least a portion of the coding tag, and a secondary tag, to allow hybridization between the adaptor molecule (or the first hybridization sequence) and the coding tag (or the portion of the coding tag); (d) transferring the information of the secondary tag to the recording tag to generate an extended recording tag; and analyzing the extended recording tag. Also provided are kits containing components and/or reagents for performing the provided methods for macromolecule sequencing and/or analysis. In some embodiments, the kits also include instructions for using the kit to perform any of the methods provided herein.

Highly-parallel characterization and recognition of macromolecules such as proteins has been challenging. Affinity-based assays may be used for this purpose but is often difficult due to several key challenges. One significant challenge is multiplexing the readout of a collection of affinity agents to a collection of cognate macromolecules, for example using affinity agents with detectable labels; another challenge is minimizing cross-reactivity between the affinity agents and off-target macromolecules; a third challenge is developing an efficient high-throughput read out platform. An example of this problem occurs in proteomics in which one goal is to identify and quantitate numerous proteins in a sample. Currently this is a formidable task to accomplish in a high-throughput way. Assays such as immunoassays and mass spectrometry based methods have been used but are limited at both the sample and analyte level, limited sensitivity and dynamic range, and cross-reactivity and background signals. Ligation-detection-reaction (LDR) based microarrays can be used for detection by utilizing the specificity of the ligase enzyme to covalently join two adjacently hybridized probes. In LDR, a zip-oligo is attached to the 3′-end of one probe and fluorescent label to the 5′-end of the other probe (See e.g., Hultman et al., BMC Microbiol. (2008) 8: 237). Other universal DNA microarray methods which use nucleic acid information transfer including polymerase chain reaction/ligase detection reaction (PCR/LDR) with “zip-code” hybridization have also been used to detect low abundance mutations (See e.g., Ladner et al., Lab Invest. (2001) 81(8):1079-86); Gerry et al., J Mol Biol (1999) 292:251-262). However, there remains a need for improved techniques relating to macromolecule sequencing and/or analysis, with applications to protein sequencing and/or analysis, as well as to products, methods and kits for accomplishing the same. There is a need for proteomics technology for performing macromolecule analysis that is flexible, highly-parallelized, accurate, sensitive, and high-throughput.

In some embodiments, the present disclosure provides, in part, methods for analyzing a macromolecule which includes information transfer, with direct applications to protein and peptide characterization, quantitation, and/or sequencing. In some examples, the information transferred comprises identifying information regarding a binding agent that is configured to bind to the macromolecule. The information transfer can be achieved by any suitable means such as by extension or ligation, and can be between nucleic acid molecules, e.g., between a nucleic acid tag associated with the binding agent and a secondary tag on an adaptor molecule. In some embodiments, provided herein are methods for transferring information from a secondary tag of an adaptor molecule to a recording tag associated with the macromolecule (e.g., polypeptide) bound by the binding agent. In some embodiments, a plurality of macromolecules obtained from a sample is analyzed. In some embodiments, the sample is obtained from a subject. In some embodiments, the macromolecule sequencing or analysis method includes using a plurality of binding agents associated with coding tags to detect a plurality of macromolecules to be analyzed.

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

An “individual” or “subject” includes a mammal. Mammals include, but are not limited to, domesticated animals (e.g., cows, sheep, cats, dogs, and horses), primates (e.g., humans and non-human primates such as monkeys), rabbits, and rodents (e.g., mice and rats). An “individual” or “subject” may include birds such as chickens, vertebrates such as fish and mammals such as mice, rats, rabbits, cats, dogs, pigs, cows, ox, sheep, goats, horses, monkeys and other non-human primates. In certain embodiments, the individual or subject is a human.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

The terms “level” or “levels” are used to refer to the presence and/or amount of a target, e.g., a substance or an organism that is part of the etiology of a disease or disorder, and can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a healthy control.

As used herein, the term “macromolecule” encompasses large molecules composed of smaller subunits. Examples of macromolecules include, but are not limited to peptides, polypeptides, proteins, nucleic acids, carbohydrates, lipids, macrocycles. A macromolecule also includes a chimeric macromolecule composed of a combination of two or more types of macromolecules, covalently linked together (e.g., a peptide linked to a nucleic acid). A macromolecule may also include a “macromolecule assembly”, which is composed of non-covalent complexes of two or more macromolecules. A macromolecule assembly may be composed of the same type of macromolecule (e.g., protein-protein) or of two more different types of macromolecules (e.g., protein-DNA).

As used herein, the term “polypeptide” encompasses peptides and proteins, and refers to a molecule comprising a chain of two or more amino acids joined by peptide bonds. In some embodiments, a polypeptide comprises 2 to 50 amino acids, e.g., having more than 20-30 amino acids. In some embodiments, a peptide does not comprise a secondary, tertiary, or higher structure. In some embodiments, the polypeptide is a protein. In some embodiments, a protein comprises 30 or more amino acids, e.g. having more than 50 amino acids. In some embodiments, in addition to a primary structure, a protein comprises a secondary, tertiary, or higher structure. The amino acids of the polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed. Polypeptides may be synthetically produced, isolated, recombinantly expressed, or be produced by a combination of methodologies as described above. Polypeptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, β-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “post-translational modification” refers to modifications that occur on a peptide after its translation, e.g., translation by ribosomes, is complete. A post-translational modification may be a covalent chemical modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini. The term post-translational modification can also include peptide modifications that include one or more detectable labels.

As used herein, the term “binding agent” refers to a nucleic acid molecule, a peptide, a polypeptide, a protein, carbohydrate, or a small molecule that binds to, associates, unites with, recognizes, or combines with a binding target, e.g., a polypeptide or a component or feature of a polypeptide. A binding agent may form a covalent association or non-covalent association with the polypeptide or component or feature of a polypeptide. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. A binding agent may bind to a single monomer or subunit of a polypeptide (e.g., a single amino acid of a polypeptide) or bind to a plurality of linked subunits of a polypeptide (e.g., a di-peptide, tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypeptide, or protein, or bind to a conformational peptide, polypeptide, or protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may preferably bind to a chemically modified or labeled amino acid (e.g., an amino acid that has been labeled by a chemical reagent) over a non-modified or unlabeled amino acid. For example, a binding agent may preferably bind to an amino acid that has been labeled or modified over an amino acid that is unlabeled or unmodified. A binding agent may bind to a post-translational modification of a peptide molecule. A binding agent may exhibit selective binding to a component or feature of a polypeptide (e.g., a binding agent may selectively bind to one of the 20 possible natural amino acid residues and bind with very low affinity or not at all to the other 19 natural amino acid residues). A binding agent may exhibit less selective binding, where the binding agent is capable of binding or configured to bind to a plurality of components or features of a polypeptide (e.g., a binding agent may bind with similar affinity to two or more different amino acid residues). A binding agent may comprise a coding tag, which may be joined to the binding agent by a linker.

As used herein, the term “linker” refers to one or more of a nucleotide, a nucleotide analog, an amino acid, a peptide, a polypeptide, a polymer, or a non-nucleotide chemical moiety that is used to join two molecules. A linker may be used to join a binding agent with a coding tag, a recording tag with a polypeptide, a polypeptide with a support, a recording tag with a solid support, etc. In certain embodiments, a linker joins two molecules via enzymatic reaction or chemistry reaction (e.g., click chemistry).

The term “ligand” as used herein refers to any molecule or moiety connected to the compounds or substances described herein. “Ligand” may refer to one or more ligands attached to a compound. In some embodiments, the ligand is a pendant group or binding site (e.g., the site to which the binding agent binds).

As used herein, the term “proteome” can include the entire set of proteins, polypeptides, or peptides (including conjugates or complexes thereof) expressed by a genome, cell, tissue, or organism at a certain time, of any organism. In one aspect, it is the set of expressed proteins in a given type of cell or organism, at a given time, under defined conditions. Proteomics is the study of the proteome. For example, a “cellular proteome” may include the collection of proteins found in a particular cell type under a particular set of environmental conditions, such as exposure to hormone stimulation. An organism's complete proteome may include the complete set of proteins from all of the various cellular proteomes. A proteome may also include the collection of proteins in certain sub-cellular biological systems. For example, all of the proteins in a virus can be called a viral proteome. As used herein, the term “proteome” include subsets of a proteome, including but not limited to a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any combination thereof. As used herein, the term “proteomics” refers to qualitative or quantitative analysis of the proteome within cells, tissues, and bodily fluids, and the corresponding spatial distribution of the proteome within the cell and within tissues. Additionally, proteomics studies include the dynamic state of the proteome, continually changing in time as a function of biology and defined biological or chemical stimuli.

The terminal amino acid at one end of a peptide or polypeptide chain that has a free amino group is referred to herein as the “N-terminal amino acid” (NTAA). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the n^{th} amino acid (also referred to herein as the “n NTAA”). Using this nomenclature, the next amino acid is the n−1 amino acid, then the n−2 amino acid, and so on down the length of the peptide from the N-terminal end to C-terminal end. In certain embodiments, an NTAA, CTAA, or both may be modified or labeled with a moiety or a chemical moiety.

As used herein, the term “barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for a polypeptide, a binding agent, a set of binding agents from a binding cycle, a sample polypeptides, a set of samples, polypeptides within a compartment (e.g., droplet, bead, or separated location), polypeptides within a set of compartments, a fraction of polypeptides, a set of polypeptide fractions, a spatial region or set of spatial regions, a library of polypeptides, or a library of binding agents. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error correcting barcodes. Barcodes can be used to computationally deconvolute the multiplexed sequencing data and identify sequence reads derived from an individual polypeptide, sample, library, etc. A barcode can also be used for deconvolution of a collection of polypeptides that have been distributed into small compartments for enhanced mapping. For example, rather than mapping a peptide back to the proteome, the peptide is mapped back to its originating protein molecule or protein complex.

As used herein, the term “coding tag” refers to a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety).

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag or adaptor molecule. In certain embodiments, a spacer sequence flanks the secondary tag sequence on one end or both ends. Following binding of a binding agent to a polypeptide, annealing between complementary spacer sequences on their associated adaptor molecule and recording tag, respectively, allows transfer of binding information through a primer extension reaction or ligation to the recording tag. Sp′ refers to spacer sequence complementary to Sp. Preferably, spacer sequences used with a library of binding agents possess the same number of bases. A common (shared or identical) spacer may be used with a library of binding agents. A spacer sequence may have a “cycle specific” sequence in order to track binding agents used in a particular binding cycle. The spacer sequence (Sp) can be constant across all binding cycles, be specific for a particular class of polypeptides, or be binding cycle number specific. Only the sequential binding of correct cognate pairs results in interacting spacer elements and effective primer extension. A spacer sequence may comprise sufficient number of bases to anneal to a complementary spacer sequence in a recording tag to initiate a primer extension (also referred to as polymerase extension) reaction, or provide a “splint” for a ligation reaction, or mediate a “sticky end” ligation reaction.

As used herein, the term “recording tag” refers to a moiety, e.g., a chemical coupling moiety, a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) to which identifying information of a coding tag can be transferred, either directly or indirectly (e.g., via an adaptor molecule). For example, information from a secondary tag of an adaptor molecule (e.g., as a proxy, representation or correlation of the information of a coding tag) can be transferred to the recording tag. In some embodiments, identifying information about the macromolecule (e.g., UMI information) associated with the recording tag can be transferred to the coding tag. Identifying information can comprise any information characterizing a molecule such as information pertaining to sample, fraction, partition, spatial location, interacting neighboring molecule(s), cycle number, etc. Additionally, the presence of UMI information can also be classified as identifying information. In certain embodiments, after a binding agent binds to a polypeptide, information from a secondary tag of the adaptor molecule can be transferred to the recording tag associated with the polypeptide. In other embodiments, after a binding agent binds to a polypeptide, information from a recording tag associated with the polypeptide can be transferred to the coding tag linked to the binding agent while the binding agent is bound to the polypeptide. A recording tag may be directly linked to a polypeptide, linked to a polypeptide via a multifunctional linker, or associated with a polypeptide by virtue of its proximity (or co-localization) on a support. A recording tag may be linked via its 5′ end or 3′ end or at an internal site, as long as the linkage is compatible with the method used to transfer information to the recording tag. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer secondary tag information to the recording tag.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases) in length providing a unique identifier tag for each macromolecule, polypeptide or binding agent to which the UMI is linked. A polypeptide UMI can be used to computationally deconvolute sequencing data from a plurality of extended recording tags to identify extended recording tags that originated from an individual polypeptide. A polypeptide UMI can be used to accurately count originating polypeptide molecules by collapsing NGS reads to unique UMIs. A binding agent UMI can be used to identify each individual molecular binding agent that binds to a particular polypeptide. For example, a UMI can be used to identify the number of individual binding events for a binding agent specific for a single amino acid that occurs for a particular peptide molecule. It is understood that when UMI and barcode are both referenced in the context of a binding agent or polypeptide, that the barcode refers to identifying information other that the UMI for the individual binding agent or polypeptide (e.g., sample barcode, compartment barcode, binding cycle barcode).

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. For example, extended recording tag molecules may be circularized and a universal priming site used for rolling circle amplification to form DNA nanoballs that can be used as sequencing templates (Drmanac et al., 2009, Science 327:78-81). Alternatively, recording tag molecules may be circularized and sequenced directly by polymerase extension from universal priming sites (Korlach et al., 2008, Proc. Natl. Acad. Sci. 105:1176-1181). The term “forward” when used in context with a “universal priming site” or “universal primer” may also be referred to as “5” or “sense”. The term “reverse” when used in context with a “universal priming site” or “universal primer” may also be referred to as “3′” or “antisense”.

As used herein, the term “extended recording tag” refers to a recording tag to which information of (or representing or correlating to) at least one binding agent has been transferred following binding of the binding agent to a polypeptide. Information may be transferred to the recording tag directly (e.g., ligation) or indirectly (e.g., primer extension) from a secondary tag of an adaptor molecule. Information of may be transferred to the recording tag enzymatically or chemically. An extended recording tag may comprise binding agent information of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 125, 150, 175, 200 or more binding agents. The base sequence of an extended recording tag may reflect the temporal and sequential order of binding of the binding agents, may reflect a partial sequential order of binding of the binding agents, or may not reflect any order of binding of the binding agents. In certain embodiments, the information of the binding agents present in the extended recording tag represents with at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity the polypeptide sequence being analyzed. In certain embodiments where the extended recording tag does not represent the polypeptide sequence being analyzed with 100% identity, errors may be due to off-target binding by a binding agent, or to a “missed” binding cycle (e.g., because a binding agent fails to bind to a polypeptide during a binding cycle, because of a failed primer extension reaction), or both.

As used herein, the term “solid support”, “solid surface”, or “solid substrate”, or “sequencing substrate”, or “substrate” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), γPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, “nucleic acid sequencing” means the determination of the order of nucleotides in a nucleic acid molecule or a sample of nucleic acid molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays (See e.g., Service, Science (2006) 311:1544-1546).

As used herein, “single molecule sequencing” or “third generation sequencing” refers to next-generation sequencing methods wherein reads from single molecule sequencing instruments are generated by sequencing of a single molecule of DNA. Unlike next generation sequencing methods that rely on amplification to clone many DNA molecules in parallel for sequencing in a phased approach, single molecule sequencing interrogates single molecules of DNA and does not require amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing (Pacific Biosciences), nanopore-based sequencing (Oxford Nanopore), duplex interrupted nanopore sequencing, and direct imaging of DNA using advanced microscopy.

As used herein, “analyzing” the polypeptide means to identify, detect, quantify, characterize, distinguish, or a combination thereof, all or a portion of the components of the polypeptide. For example, analyzing a peptide, polypeptide, or protein includes determining all or a portion of the amino acid sequence (contiguous or non-continuous) of the peptide. Analyzing a polypeptide also includes partial identification of a component of the polypeptide. For example, partial identification of amino acids in the polypeptide protein sequence can identify an amino acid in the protein as belonging to a subset of possible amino acids. Analysis typically begins with analysis of the n NTAA, and then proceeds to the next amino acid of the peptide (i.e., n−1, n−2, n−3, and so forth). This is accomplished by removing the n NTAA, thereby converting the n−1 amino acid of the peptide to an N-terminal amino acid (referred to herein as the “n−1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

It is understood that aspects and embodiments of the invention described herein include “consisting of” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### I. INFORMATION TRANSFER

Provided herein are methods and kits for analysis of macromolecules, e.g., peptides, polypeptides, and proteins, which includes a step of transferring information to a recording tag. In some embodiments, the analysis employs barcoding and nucleic acid encoding of molecular recognition events, and/or detectable labels. In some examples, the information transferred comprises identifying information regarding a binding agent that is configured to bind to the macromolecule. The information transfer can be achieved by any suitable means such as by extension or ligation, and can be between nucleic acid molecules, e.g., between a nucleic acid tag associated with the macromolecule for analysis and a secondary tag on an adaptor molecule. The provided method for information transfer comprises: (a) providing a macromolecule and an associated recording tag joined to a support; (b) contacting the macromolecule with a binding agent capable of binding to the macromolecule, wherein the binding agent comprises a coding tag with identifying information regarding the binding agent, to allow binding between the macromolecule and the binding agent; (c) providing an adaptor molecule comprising a first hybridization sequence substantially complementary to at least a portion of the coding tag (or portion thereof), and a secondary tag, to allow hybridization between the adaptor molecule (or the first hybridization sequence) and the coding tag (or the portion of the coding tag); (d) transferring the information of the secondary tag to the recording tag to generate an extended recording tag; and analyzing the extended recording tag. The methods provided herein may include providing a plurality of binding agents and a plurality of macromolecules and allowing the binding agents and macromolecules to interact. In some embodiments, a plurality of adaptor molecules are provided. In some embodiments, the present methods comprise contacting a single macromolecule with a single binding agent, contacting a plurality of macromolecules with a single binding agent, or contacting a plurality of macromolecules with a plurality of binding agents.

In a preferred embodiment, macromolecule comprises a polypeptide.

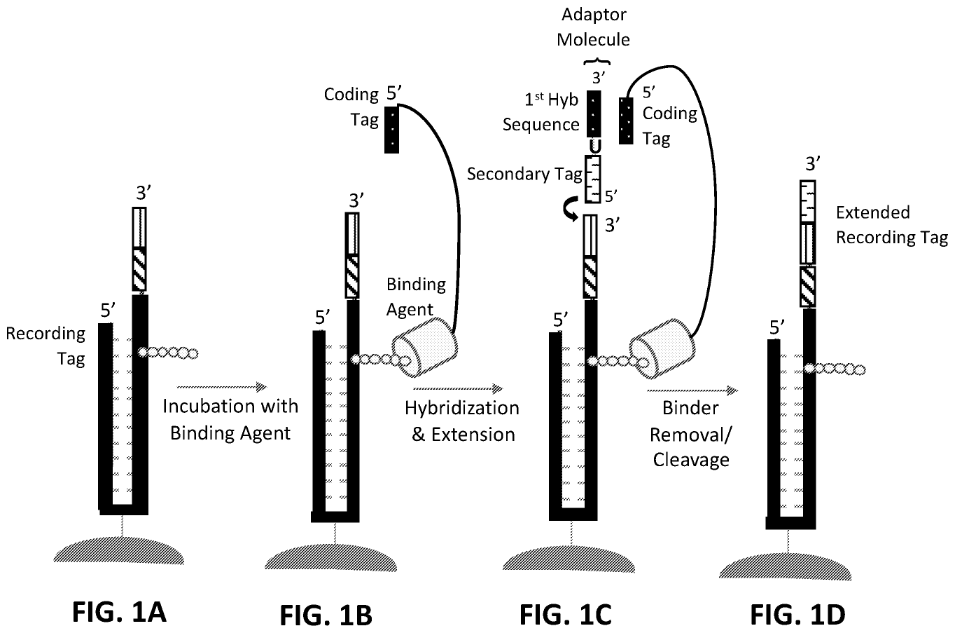

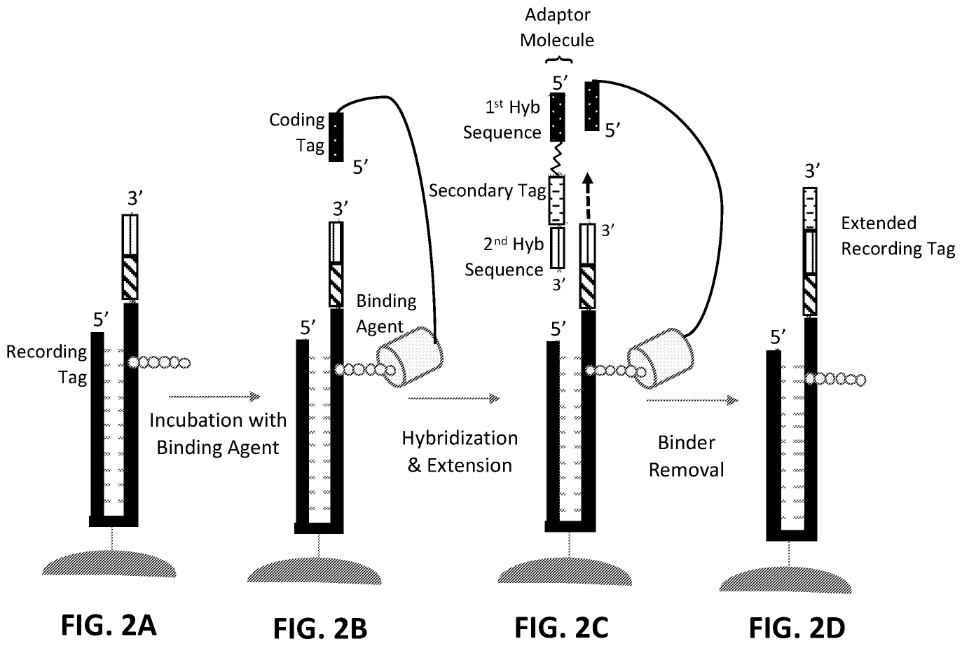

In some embodiments, the present disclosure provides, in part, methods for analyzing a macromolecule which includes information transfer, with direct applications to protein and peptide characterization, quantitation, and/or sequencing. Provided herein are methods for transferring information from a secondary tag of an adaptor molecule to a recording tag associated with the macromolecule (e.g., polypeptide) bound by the binding agent. Transfer of information may be performed via ligation, extension or other methods known in the art. The information transferred from the secondary tag of an adaptor molecule includes identifying information regarding the identity of the binding agent, the macromolecule or portion thereof bound by the binding agent. For example, if a protein macromolecule is bound by the binding agent, the identifying information may comprise information regarding the identity of the one or more amino acid(s) on the peptide bound by the binding agent (see FIG. 1A-1D and FIG. 2A-2D). In some embodiments, the information regarding the identity of the macromolecule bound by the binding agent is from the coding tag associated with said binding agent, and transferred to the recording tag via the hybridized adaptor molecule. The macromolecule analysis assay may include one or more cycles of transferring identifying information of a binding agent to a recording tag associated with the macromolecule to be analyzed. The extended recording tag associated with the macromolecule for analysis can comprise the information from one or more secondary tags. If multiple cycles are performed, the resulting extended recording tag then contains information built up from a series of binding events and multiple information transfer events using adaptor molecules comprising secondary tags. In general, improvements for the transfer of information may provide certain benefits to the macromolecule analysis assay.

In particular, the adaptor molecules provided in step (c) used in this method for analyzing macromolecules provides certain advantages to the overall design of the assay. In this system, the adaptor molecule serves as an intermediate between the information on the coding tag associated with the binding agent and the transferred information on the recording tag. The adaptor molecules comprise a first hybridization sequence and a secondary tag, wherein the first hybridization sequence or portion thereof is substantially complementary or complementary to the coding tag or a region therein. The first hybridization sequence allows each coding tag to be associated with an adaptor molecule and its contained secondary tag. In some aspects, the use of the adaptor molecules provides the ability to adjust the information transferred to the recording tag (via the secondary tag) quickly and conveniently, by obviating the need to remake binding agent-coding tag conjugates, which may be a time consuming process. In some cases, the use of the adaptor molecule provides some flexibility such as the ability to collapse information at the level of the secondary tag. In some embodiments, the adaptor molecules may be designed to contain suitable barcodes (e.g. as part of the secondary tag) based on the sequencing system used for the readout. For example, the barcode may be suitable for less accurate NGS such as nanopore sequencing (e.g., more error correction).

In some embodiments related, for example, to polypeptide sequencing, multiple cycle of transferring information from a binding agent-fused coding tag to the polypeptide-associated recording tag occur, similar to the cycles shown in FIG. 1 and FIG. 2. In preferred embodiments, at the end of each cycle, the terminal amino acid of the polypeptide gets cleaved off, so the next amino acid of the polypeptide becomes a new terminal amino acid and a target for binding agents on the next cycle.

Provided herein are methods for transferring information from a secondary tag of an adaptor molecule to a recording tag associated with the macromolecule (e.g., polypeptide) bound by the binding agent. Transfer of information may be performed via ligation, extension or other methods known in the art. The information transferred from the secondary tag of an adaptor molecule includes identifying information regarding the identity of the binding agent, the macromolecule or portion thereof bound by the binding agent. For example, if a protein macromolecule is bound by the binding agent, the identifying information may comprise information regarding the identity of the one or more amino acid(s) on the peptide bound by the binding agent

For the multicycle ProteoCode assay, which can comprise >15 cycles of cycle-specific binding and encoding events, the use of cycle-specific adapter molecules rather than cycle-specific DNA-tagged binding agents recognizing a particular NTAA greatly decreases the manufacturing burden of creating and maintaining a set of 20 or more binders (this set would recognize all 20 NTAA and/or post-translationally modified NTAA) multiply by 15+ cycles. For 20 binders and 15 cycles, this equates to a set of 300 binders. Rather, than a new pool of binders for each cycle, a new pool of cycle-specific adapter molecules can be used to translate NTAA binding information into cycle-specific NTAA binding information. As such, a single universal set of 15-20 DNA-conjugated binding agents would be employed in each cycle, and cycle-specific information would be conferred by the use of cycle-specific pools of adapter molecules.

The analysis assay includes the use of a plurality of binding agents and each binding agent is associated with a coding tag containing identifying information regarding the identity of the macromolecule. An example of collapsing information from multiple binding agents is as follows: the macromolecule “X” may be bound by two binding agents (such as at different motifs on the molecule) with corresponding coding tags B1 and B2, two adaptor molecules which contain a secondary tag and B1′ and B2′ as the first hybridization region, respectively, can both be associated with “X”. Thus the information from two binding agents is collapsed at the level of the secondary tag and transferred as the same information to the recording tag. In this case, the option also remains to switch in adaptor molecules that retain the information of the B1 and B2 binding agents while using the same binding agents and associated coding tags, simply by using different adaptor molecules. In some cases, the adaptor molecules can be exchanged or modified without the need for modifying the binding agent and associated coding tags.

In some embodiments, a spacer is added to the end of the recording tag with the secondary tag, and the spacer comprises a sequence that is capable of hybridizing with a sequence on the adaptor molecule to facilitate transfer of the identifying information.

In some embodiments, the adaptor molecule further comprises a second hybridization sequence substantially complementary or complementary to a portion of the recording tag. The second hybridization sequence on the adaptor molecule may be substantially complementary or complementary to a sequence at the 3′ terminus of the recording tag, such as a region on the recording tag generated from a previous information transfer of the secondary tag from the adaptor molecule to the recording tag. In some cases, information transfer of the secondary tag from the adaptor molecule to the recording tag occurs if both the first hybridization sequence on the adaptor molecule hybridizes to the coding tag of a binding agent and the second hybridization sequence of the adaptor molecule hybridizes to a portion of the recording tag. In this case, a set of adaptor molecules is used such that combinations of the first hybridization sequence and the second hybridization sequence covers all combinations needed to hybridize to potential complementary sequences on the recording tags and coding tags. In some embodiments, the provided method for information transfer using the adaptor molecule comprising a second hybridization sequence enables a “spacer-less” approach of transferring information from an adaptor molecule to a recording tag. For example, this may remove the need for inserting a spacer sequence for hybridization purposes into the extended recording tag. In some cases, since each cycle of the method includes extending the recording tag, a spacer-less approach provides the benefit that the extended recording tag length can be reduced.

Identifying information associated with a specific binding agent may be transferred to a recording tag using a variety of methods. The transfer in the methods provided herein are from the secondary tag of an adaptor molecule to the recording tag to generate an extended recording tag. In some embodiments, the transfer of identifying information (e.g., from a secondary tag to a recording tag) can be accomplished by ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof.

Identifying information associated with a specific binding agent may be transferred to a nucleic acid on the recording tag associated with the immobilized macromolecule via ligation (FIG. 1A-1D). Ligation may be a blunt end ligation or sticky end ligation. Ligation may be an enzymatic ligation reaction. Examples of ligases include, but are not limited to CV DNA ligase, T4 DNA ligase, T7 DNA ligase, T3 DNA ligase, Taq DNA ligase, E. coli DNA ligase, 9° N DNA ligase (See e.g., U.S. Patent Publication No. US20140378315). After ligation of the secondary tag to the recording tag, the other portions of the adaptor molecule (e.g., the first hybridization sequence) if no longer needed may be cleaved and released. In some embodiments, the adaptor molecule comprises one or more uracil bases, which can be targeted for digestion with a uracil-specific excision reagent (e.g., USER™).

In another embodiment, transfer of PNAs can be accomplished with chemical ligation using published techniques. The structure of PNA is such that it has a 5′ N-terminal amine group and an unreactive 3′ C-terminal amide. Chemical ligation of PNA requires that the termini be modified to be chemically active. This is typically done by derivatizing the 5′ N-terminus with a cysteinyl moiety and the 3′ C-terminus with a thioester moiety. Such modified PNAs easily couple using standard native chemical ligation conditions (Roloff et al., (2013) Bioorgan. Med. Chem. 21:3458-3464).

In some embodiments, identifying information from a secondary tag can be transferred to a recording tag using topoisomerase. Topoisomerase can be used be used to ligate a topo-charged 3′ phosphate on the recording tag (or extensions thereof or any nucleic acids attached) to the 5′ end of the coding tag, or complement thereof (Shuman et al., 1994, J. Biol. Chem. 269:32678-32684).

In certain embodiments, information is transferred to a recording tag via primer extension (Chan et al. (2015) Curr Opin Chem Biol 26: 55-61). A sequence on the 3′-terminus of a recording tag or an extended recording tag anneals with complementary sequence on the 3′ terminus of an adaptor molecule and a polymerase (e.g., strand-displacing polymerase) extends the recording tag sequence, using the secondary tag of the annealed adaptor molecule as a template (FIG. 2A-2D). In some cases, the complementary sequence on the 3′ terminus of an adaptor molecule may be a spacer sequence. In some cases, the complementary sequence on the 3′ terminus of an adaptor molecule may be the second hybridization sequence. In some examples, the adaptor molecule comprises a spacer or linker to stop extension after transfer of information from the secondary tag to the recording tag.

In some embodiments, a DNA polymerase that is used for primer extension possesses strand-displacement activity and has limited or is devoid of 3′-5 exonuclease activity. Several of many examples of such polymerases include Klenow exo- (Klenow fragment of DNA Pol 1), T4 DNA polymerase exo-, T7 DNA polymerase exo (Sequenase 2.0), Pfu exo-, Vent exo-, Deep Vent exo-, Bst DNA polymerase large fragment exo-, Bca Pol, 9° N Pol, and Phi29 Pol exo-. In a preferred embodiment, the DNA polymerase is active at room temperature and up to 45° C. In another embodiment, a “warm start” version of a thermophilic polymerase is employed such that the polymerase is activated and is used at about 40° C.-50° C. An exemplary warm start polymerase is Bst 2.0 Warm Start DNA Polymerase (New England Biolabs).

Additives useful in strand-displacement replication include any of a number of single-stranded DNA binding proteins (SSB proteins) of bacterial, viral, or eukaryotic origin, such as SSB protein of E. coli, phage T4 gene 32 product, phage T7 gene 2.5 protein, phage Pf3 SSB, replication protein A RPA32 and RPA14 subunits (Wold, Annu. Rev. Biochem. (1997) 66:61-92); other DNA binding proteins, such as adenovirus DNA-binding protein, herpes simplex protein ICP8, BMRF1 polymerase accessory subunit, herpes virus UL29 SSB-like protein; any of a number of replication complex proteins known to participate in DNA replication, such as phage T7 helicase/primase, phage T4 gene 41 helicase, E. coli Rep helicase, E. coli recBCD helicase, recA, E. coli and eukaryotic topoisomerases (Annu Rev Biochem. (2001) 70:369-413).

Mis-priming or self-priming events, such as when the terminal spacer sequence of the recording tag primes extension self-extension may be minimized by inclusion of single stranded binding proteins (T4 gene 32, E. coli SSB, etc.), DMSO (1-10%), formamide (1-10%), BSA(10-100 ug/ml), TMACl (1-5 mM), ammonium sulfate (10-50 mM), betaine (1-3 M), glycerol (5-40%), or ethylene glycol (5-40%), in the primer extension reaction.

Most type A polymerases devoid of 3′ exonuclease activity (endogenous or engineered removal), such as Klenow exo-, T7 DNA polymerase exo- (Sequenase 2.0), and Taq polymerase catalyze non-templated addition of a nucleotide, preferably an adenosine base (to lesser degree a G base, dependent on sequence context) to the 3′ blunt end of a duplex extension product. For Taq polymerase, a 3′ pyrimidine (C>T) minimizes non-templated adenosine addition, whereas a 3′ purine nucleotide (G>A) favours non-templated adenosine addition. Alternatively, addition of non-templated base can be reduced by employing a mutant polymerase (mesophilic or thermophilic) in which non-templated terminal transferase activity has been greatly reduced by one or more point mutations, especially in the 0-helix region (see U.S. Pat. No. 7,501,237) (Yang et al., Nucleic Acids Res. (2002) 30(19): 4314-4320). Pfu exo-, which is 3′ exonuclease deficient and has strand-displacing ability, also does not have non-templated terminal transferase activity.

In another embodiment, polymerase extension buffers are comprised of 40-120 mM buffering agent such as Tris-Acetate, Tris-HCl, HEPES, etc. at a pH of 6-9.

Self-priming/mis-priming events initiated by self-annealing of the terminal spacer sequence of the extended recording tag with internal regions of the extended recording tag may be minimized by including pseudo-complementary bases in the recording/extended recording tag (Lahoud, Timoshchuk et al. 2008), (Hoshika, Chen et al. 2010). Pseudo-complementary bases show significantly reduced hybridization affinities for the formation of duplexes with each other due the presence of chemical modification. However, many pseudo-complementary modified bases can form strong base pairs with natural DNA or RNA sequences. In certain embodiments, the coding tag spacer sequence is comprised of multiple A and T bases, and commercially available pseudo-complementary bases 2-aminoadenine and 2-thiothymine are incorporated in the recording tag using phosphoramidite oligonucleotide synthesis. Additional pseudocomplementary bases can be incorporated into the extended recording tag during primer extension by adding pseudo-complementary nucleotides to the reaction (Gamper, Arar et al. 2006).

In certain embodiments, the binding event information of the binding agent to the macromolecule (e.g., peptide) is transferred from a secondary tag of an adaptor molecule to the recording tag associated with the immobilized macromolecule in a cyclic fashion. In some embodiments, steps repeated one or more times include: (b) contacting the macromolecule with a binding agent capable of binding to the macromolecule, wherein the binding agent comprises a coding tag with identifying information regarding the binding agent; (c) providing an adaptor molecule comprising a first hybridization sequence substantially complementary or complementary to at least a portion of the coding tag or the entire coding tag, and a secondary tag; and (d) transferring the information of the secondary tag to the recording tag to generate an extended recording tag. In some cases, the method further includes one or more wash steps between any of steps (b), (c), and (d). In some cases, the method further includes removing the binding agent and/or the adaptor molecule.

In some embodiments, a set of adaptor molecules used in a cycle of binding and information transfer may include cycle information, such as using cycle specific sequences. In one embodiment, the adaptor molecules comprise binding cycle-specific sequences. In one embodiment, the secondary tags comprise binding cycle-specific sequences. Binding cycle-specific sequences may be accomplished either via the use of completely unique binding cycle barcodes or through unique combinations of sub-barcodes. In some aspects, embedding binding cycle information directly in the secondary tag sequence may allow the total length of the coding tag to be minimized when employing error-correcting barcodes. The use of error-tolerant barcodes allows highly accurate barcode identification using sequencing platforms and approaches that are more error-prone, but have other advantages such as rapid speed of analysis, lower cost, and/or more portable instrumentation.

In some aspects, the provided methods also allow the use of adaptor molecules with secondary tags that are of preferred lengths, such as a length suitable for a particular sequencing method. In some embodiments, adaptor molecules may comprise secondary tags containing error-tolerant barcodes. In some embodiments, various libraries or sets of adaptor molecules can be designed to be compatible with a particular sequencing method and switched interchangeably if another sequencing method is preferred for the downstream analysis step.

A. Adaptor Molecules

Provided herein are methods for analyzing a macromolecule including use of an adaptor molecule comprising a first hybridization sequence and a secondary tag. The methods provided may include preparing, selecting, and providing a single adaptor molecule or a plurality of adaptor molecules. The library or set of adaptor molecules used in the provided methods comprises at least one adaptor molecule configured to hybridize to at least one coding tag associated with the binding agents used for analyzing the macromolecule (or a portion of the coding tag). In some embodiments, the adaptor molecule further comprises a second hybridization sequence. The first hybridization sequence comprises a sequence substantially complementary or complementary to particular coding tags, allowing adaptor molecules to hybridize to corresponding coding tags and associate the secondary tag with the corresponding binding agents. In some embodiments, the information from the secondary tag can be transferred from the adaptor molecule to the recording tag after the coding tag associated with the binding agent hybridizes with the first hybridization sequence on the adaptor molecule. The adaptor molecule may comprise any suitable nucleic acid molecule including a DNA molecule, an RNA molecule, a PNA molecule, a BNA molecule, an XNA, molecule, an LNA molecule, a γPNA molecule, or a combination thereof. In some examples, the adaptor molecule comprises a spacer or linker. In some examples, the linker or spacer is provided to stop extension after transfer of information from the secondary tag to the recording tag. In some examples, a linker may be a flexible linker (e.g., PEG linker). In some embodiments, the adaptor molecule may further comprise a universal priming site, a binding cycle specific spacer, binding cycle-specific barcode, a UMI or any combination thereof.

In some embodiments, multiple coding tags associated with a binding agent is configured to hybridize to adaptor molecules comprising the same secondary tag. For example, two binding agents that provide the same identifying information regarding a binding agent and the corresponding cognate target may be designed such that each binding agent is associated with the same secondary tag. In some examples for analyzing peptides, a binding agent that binds peptides with a terminal alanine (P1)-alanine (P2) and a binding agent that binds peptides with a terminal alanine (P1)-arginine (P2) both provide information that a peptide has a NTAA (P1) that is alanine, regardless of the amino acid at the penultimate position (P2). The coding tag associated with both binding agents hybridize with adaptor molecules which share the same secondary tag providing information of the terminal alanine on the peptide, thus collapsing the information of multiple binders into one piece of information at the level of the secondary tag.

In some embodiments, the first hybridization sequence (1^{st} Hyb Sequence in FIGS. 1C and 2C) of the adaptor molecule comprises a single stranded region for hybridizing to the coding tag (or region therein) associated with the binding agent. In some embodiments, the first hybridization sequence comprises at least one nucleic acid region which is substantially complementary to a coding tag or portion thereof. In some embodiments, the first hybridization sequence in the adaptor molecule is complementary to a portion of the coding tag. In some embodiments, the first hybridization sequence in the adaptor molecule is complementary to the entire coding tag. In some examples, the first hybridization sequence comprises a sequence of nucleotides that binds selectively to the coding tag sequence or portion thereof. In some embodiments, the first hybridization sequence comprises a single stranded region which is substantially complementary to the coding tag sequence. “Substantially complementary” refers to sequences that are capable of hybridizing to a target nucleic acid sequence under the conditions employed. In preferred embodiments, a “substantially complementary” single-stranded region is exactly complementary to a target nucleic acid sequence. For example, the single-stranded region of the first hybridization sequence complementary to the coding tag may have at least 4 bases, at least 5 bases, at least 6 bases, at least 7 bases, at least 8 bases, at least 9 bases, at least 10 bases, at least 12 bases, at least 14 bases, at least 16 bases, at least 20 bases, at least 24 bases, at least 30 bases, or at least 34 bases. In some embodiments, the single-stranded region of the first hybridization sequence complementary to the coding tag has fewer than 40 bases, fewer than 30 bases, or fewer than 25 bases. One skilled in the art may select complementary regions with number of bases that is sufficient for forming stable hybridization regions between the first hybridization sequence and the coding tag. In some embodiments, the first hybridization sequence for hybridizing to the coding tag is located at the 3′ or the 5′ terminus of the adaptor molecule. In some specific embodiments, the first hybridization sequence for hybridizing to the coding tag is located at the 5′ terminus of the adaptor molecule. In some examples, the first hybridization sequence is 5′ to the secondary tag in the adaptor molecule.

In some embodiments, the second hybridization sequence (2^{nd} Hyb Sequence in FIG. 2C) of the adaptor molecule comprises a single stranded region for hybridizing to a portion of the recording tag associated with the macromolecule for analysis. For example, the second hybridization sequence on the adaptor molecule is substantially complementary or complementary to a region on the recording tag generated from a previous information transfer of the secondary tag from the adaptor molecule to the recording tag. In some aspects, the second hybridization sequence on the adaptor molecule comprises the secondary tag information or portion thereof on a different adaptor molecule. In some embodiments, the second hybridization sequence comprises at least one nucleic acid region which is substantially complementary to a portion of the recording tag or portion thereof. In some embodiments, the second hybridization sequence is substantially complementary or complementary to a sequence at the 3′ terminus of the recording tag. In some examples, the second hybridization sequence comprises a sequence of nucleotides that binds selectively to a portion of the recording tag associated with the macromolecule for analysis. In some embodiments, the second hybridization sequence comprises a single stranded region which is substantially complementary to a portion of the recording tag associated with the macromolecule for analysis. For example, the single-stranded region of the second hybridization sequence complementary to the recording tag or portion thereof may have at least 4 bases, at least 5 bases, at least 6 bases, at least 7 bases, at least 8 bases, at least 9 bases, at least 10 bases, at least 12 bases, at least 14 bases, at least 16 bases, at least 20 bases, at least 24 bases, at least 30 bases, or at least 34 bases. In some embodiments, the single-stranded region of the second hybridization sequence complementary to the recording tag or portion thereof has fewer than 40 bases, fewer than 30 bases, or fewer than 25 bases. One skilled in the art may select complementary regions with number of bases that is sufficient for forming stable hybridization regions between the second hybridization sequence and the portion of the recording tag. In some embodiments, the second hybridization sequence for hybridizing to the portion of the recording tag is located at the 3′ or the 5′ terminus of the adaptor molecule. In some specific embodiments, the second hybridization sequence is located at the 3′ terminus of the adaptor molecule.

The secondary tag of an adaptor molecule can be a polynucleotide of any suitable length, e.g., a nucleic acid molecule of about 2 bases to about 100 bases, including any integer including 2 and 100 and in between, that comprises identifying information for a binding agent. Information of a secondary tag can be associated to a binding agent via the first hybridization sequence (which is substantially complementary or complementary to the coding tag (or portion thereof) associated with a binding agent) comprised by the same adaptor molecule. A secondary tag can made from a sequenceable polymer. The secondary tag may be optionally flanked by one spacer on one side or optionally flanked by a spacer on each side. A secondary tag may also be comprised of an optional UMI and/or an optional binding cycle-specific barcode. In certain embodiments, a secondary tag may be flanked by a binding cycle specific spacer. A secondary tag may be single stranded or double stranded. A double stranded secondary tag may comprise blunt ends, overhanging ends, or both. A secondary tag may refer to the secondary tag that is part of the adaptor molecule or to a complementary sequence that is capable of hybridizing to the secondary tag, or to the information present in an extended recording tag transferred from the secondary tag. In certain embodiments, a secondary tag may further comprise a binding cycle specific barcode, a unique molecular identifier, or both. In some specific embodiments, the secondary tag is a binding cycle specific sequence. For example, one set or plurality of adaptor molecules is used with a first cycle and a second set or plurality of adaptor molecules is used with a second cycle, etc.

In some aspects, a secondary tag comprises a sequence that provides identifying information regarding the binding agent associated via the first hybridization region on the adaptor molecule. The sequence providing identifying information is about 3 bases to about 30 bases, about 3 bases to about 20 bases, about 3 bases to about 10 bases, or about 3 bases to about 8 bases. In some embodiments, an sequence providing identifying information is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. The length of the sequence providing identifying information may be adjusted based on the size of the binding agent library used.

In some embodiments, each unique binding agent within a library of binding agents is associated with a sequence providing identifying information regarding the binding agent. The secondary tag comprises this sequence providing identifying information regarding the binding agent. For example, 20 unique sequences may be used as secondary tags for providing identifying information for a library of 20 binding agents that bind to the 20 standard amino acids. Additional sequences may be used to identify modified amino acids (e.g., post-translationally modified amino acids). In another example, 30 unique sequence may be used as secondary tags for a library of 30 binding agents that bind to the 20 standard amino acids and 10 post-translational modified amino acids (e.g., phosphorylated amino acids, acetylated amino acids, methylated amino acids). In other embodiments, two or more different binding agents may be associated with the same sequence (secondary tags) providing identifying information regarding the binding agent. In some cases, 20 unique sequences may be used as secondary tags for a library of 30 binding agents that bind to the 20 standard amino acids in an overlapping manner.

In some embodiments, the secondary tag sequence can be optimized for a particular sequencing analysis platform. In a particular embodiment, the sequencing platform is nanopore sequencing. In some embodiments, the sequencing platform has a per base error rate of >1%, >5%, >10%, >15%, >20%, >25%, or >30%. For example, if the extended nucleic acid is to be analyzed using a nanopore sequencing instrument, the barcode sequences (e.g., sequences comprising information from the secondary tag) can be designed to be optimally electrically distinguishable in transit through a nanopore. In some embodiments, the length of the barcode sequences is optimally designed to implement an appropriate level of error detection and/or correction.

In certain embodiments, the adaptor molecule further comprises a spacer sequence at one end or both ends. In some embodiments, the adaptor molecule comprises a spacer at the 3′ terminus. A spacer sequence is about 1 base to about 20 bases, about 1 base to about 10 bases, about 5 bases to about 9 bases, or about 4 bases to about 8 bases. In some embodiments, a spacer is about 1 base, 2 bases, 3 bases, 4 bases, 5 bases, 6 bases, 7 bases, 8 bases, 9 bases, 10 bases, 11 bases, 12 bases, 13 bases, 14 bases, 15 bases or 20 bases in length. In some embodiments, a spacer is shorter than the sequence providing identifying information comprised in the secondary tag, e.g., at least 1 base, 2, bases, 3 bases, 4 bases, 5 bases, 6, bases, 7 bases, 8 bases, 9 bases, 10 bases, 11 bases, 12 bases, 13 bases, 14 bases, 15 bases, 20 bases, or 25 bases shorter than the sequence providing identifying information comprised in the secondary tag. In other embodiments, a spacer is the same length as the sequence providing identifying information comprised in the secondary tag. In certain embodiments, the spacer is specific to the adaptor molecule. In some cases, a spacer is designed such that a spacer from a previous binding cycle only interacts with a spacer from the appropriate adaptor molecule in a current binding cycle. A spacer sequence may be used as the primer annealing site for a primer extension reaction, or a splint or sticky end in a ligation reaction. A 5′ spacer on an adaptor molecule may optionally contain pseudo complementary bases to a 3′ spacer on the recording tag to increase T_{M} (Lehoud et al., 2008, Nucleic Acids Res. 36:3409-3419). In other embodiments, the adaptor molecules do not have a binding cycle specific spacer sequence. In some embodiments, the adaptor molecules do not have a spacer sequence.

In some embodiments, a set (e.g. a library) or plurality of adaptor molecules share a common spacer sequence used in an assay (e.g. the entire library of adaptor molecules used in a multiple binding cycle method possess a common spacer). In another embodiment, the adaptor molecules are comprised of a binding cycle tags, identifying a particular binding cycle. In other embodiments, the adaptor molecules within a library or set of adaptor molecules have a binding cycle specific spacer sequence. In some embodiments, adaptor molecules comprises one binding cycle specific spacer sequence. For example, adaptor molecules used in the first binding cycle comprise a “cycle 1” specific spacer sequence, adaptor molecules used in the second binding cycle comprise a “cycle 2” specific spacer sequence, and so on up to “n” binding cycles. In some embodiments, a spacer sequence comprises a sufficient number of bases to anneal to a complementary spacer sequence in a recording tag or extended recording tag to initiate a primer extension reaction or sticky end ligation reaction.

In some embodiments, adaptor molecules used to bind in an alternating cycles comprises different binding cycle specific spacer sequences. For example, adaptor molecules used in the first binding cycle comprise a “cycle 1” specific spacer sequence, adaptor molecules used in the second binding cycle comprise a “cycle 2” specific spacer sequence, adaptor molecules used in the third binding cycle also comprises the “cycle 1” specific spacer sequence, adaptor molecules used in the fourth binding cycle comprises the “cycle 2” specific spacer sequence. In this manner, alternating spacers can be used and cycle specific spacers are not needed for every cycle.

The adaptor molecules may also be designed to contain palindromic sequences. Inclusion of a palindromic sequence into the adaptor molecule allows a nascent, growing, extended recording tag to fold upon itself as information is transferred from the secondary tag. The extended recording tag is folded into a more compact structure, effectively decreasing undesired inter-molecular binding and primer extension events.

The adaptor molecules may include a terminator nucleotide incorporated at the 3′ end of the 3′ spacer sequence. After a binding agent binds to a polypeptide, an adaptor molecule hybridizes, and their corresponding hybridization sequences and recording tags anneal via complementary spacer sequences, it is possible for primer extension to transfer information from the secondary tag to the recording tag. Addition of a terminator nucleotide on the 3′ end of the secondary tag prevents transfer of recording tag information to the secondary tag.

In some specific embodiments, the adaptor molecule comprises from 5′ to 3′ direction the secondary tag and the first hybridization sequence, optionally with a linker in between said components. In some specific embodiments, the adaptor molecule comprises from 5′ to 3′ direction: the first hybridization sequence, the secondary tag, and the second hybridization sequence. In some specific embodiments, the adaptor molecule comprises from 5′ to 3′ direction: the first hybridization sequence, the secondary tag, and a spacer sequence. In some specific embodiments, the adaptor molecule comprises from 5′ to 3′ direction: the first hybridization sequence, a spacer or linker to stop extension, the secondary tag, and the second hybridization sequence. In some specific embodiments, the adaptor molecule comprises from 5′ to 3′ direction: the first hybridization sequence, a spacer or linker to stop extension, the secondary tag, and a spacer sequence.

In some embodiments, the method further includes adding a universal priming site from an adaptor molecule to the extended recording tag, prior to or during the last binding cycle. In some embodiments, the universal reverse priming site is added to the recording tag (e.g., extended recording tag) from an adaptor molecule. The adaptor molecules used in the final binding cycle may comprise a universal priming site. After transfer of the final secondary tag information to the extended recording tag, the tag can be capped by addition of a universal reverse priming site via ligation, primer extension or other methods known in the art. In some embodiments, a capping reaction may be performed in any binding cycle to add a capping sequence (e.g., a universal priming site) to recording tags that did not extend with information from a secondary tag in that cycle. In some cases, such a step is useful to indicate that an information transfer event did not happen in a particular binding cycle.

B. Recording Tag

In some embodiments, the macromolecule (e.g., protein or polypeptide) for analysis may be labeled with a nucleic acid molecule or an oligonucleotide (e.g., DNA recording tag). In some aspects, a plurality of macromolecules in the sample is provided with recording tags. The recording tags may be associated or attached, directly or indirectly to the macromolecules using any suitable means. In some embodiments, a macromolecule may be associated with one or more recording tags. In some aspects, the recording tag may be any suitable sequenceable moiety to which identifying information can be transferred (e.g., information from one or more secondary tags). In some aspects, the recording tags may be associated or attached, directly or indirectly to the macromolecules prior to contacting with a binding agent.

In some embodiments, at least one recording tag is associated or co-localized directly or indirectly with the macromolecule (e.g., polypeptide). In a particular embodiment, a single recording tag is attached to a polypeptide, such as via the attachment to a N- or C-terminal amino acid. In another embodiment, multiple recording tags are attached to the polypeptide, such as to the lysine residues or peptide backbone. In some embodiments, a polypeptide labeled with multiple recording tags is fragmented or digested into smaller peptides, with each peptide labeled on average with one recording tag.

A recording tag may comprise DNA, RNA, or polynucleotide analogs including PNA, gPNA, GNA, HNA, BNA, XNA, TNA, or a combination thereof. A recording tag may be single stranded, or partially or completely double stranded. A recording tag may have a blunt end or overhanging end. In certain embodiments, all or a substantial amount of the macromolecules (e.g., at least 50%, 55%, 60%, 65%, 70%, 75%, 80%, 85%, 90%, 95%, 96%, 97%, 98%, 99%, or 100%) within a sample are labeled with a recording tag. In other embodiments, a subset of macromolecules within a sample are labeled with recording tags. In a particular embodiment, a subset of macromolecules from a sample undergo targeted (analyte specific) labeling with recording tags. For example, targeted recording tag labeling of proteins may be achieved using target protein-specific binding agents (e.g., antibodies, aptamers, etc.). In some embodiments, the recording tags are attached to the macromolecules prior to providing the sample on a support. In some embodiments, the recording tags are attached to the macromolecules after providing the sample on the support.

In some embodiments, the recording tag may comprise other nucleic acid components. In some embodiments, the recording tag may comprise a unique molecular identifier, a compartment tag, a partition barcode, sample barcode, a fraction barcode, a spacer sequence, a universal priming site, or any combination thereof. In some embodiments, the recording tag may comprise a blocking group, such as at the 3′-terminus of the recording tag. In some cases, the 3′-terminus of the recording tag is blocked to prevent extension of the recording tag by a polymerase.

In some embodiments, the recording tag can include a sample identifying barcode. A sample barcode is useful in the multiplexed analysis of a set of samples in a single reaction vessel or immobilized to a single solid substrate or collection of solid substrates (e.g., a planar slide, population of beads contained in a single tube or vessel, etc.). For example, macromolecules from many different samples can be labeled with recording tags with sample-specific barcodes, and then all the samples pooled together prior to immobilization to a support, cyclic binding of the binding agent, and recording tag analysis. Alternatively, the samples can be kept separate until after creation of a DNA-encoded library, and sample barcodes attached during PCR amplification of the DNA-encoded library, and then mixed together prior to sequencing. This approach could be useful when assaying analytes (e.g., proteins) of different abundance classes.

In certain embodiments, a recording tag comprises an optional, unique molecular identifier (UMI), which provides a unique identifier tag for each macromolecules (e.g., polypeptide) to which the UMI is associated with. A UMI can be about 3 to about 40 bases, about 3 to about 30 bases, about 3 to about 20 bases, or about 3 to about 10 bases, or about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases in length. A UMI can be used to de-convolute sequencing data from a plurality of extended recording tags to identify sequence reads from individual macromolecules. In some embodiments, within a library of macromolecules, each macromolecule is associated with a single recording tag, with each recording tag comprising a unique UMI. In other embodiments, multiple copies of a recording tag are associated with a single macromolecule, with each copy of the recording tag comprising the same UMI. In some embodiments, a UMI has a different base sequence than the spacer or secondary tags to facilitate distinguishing these components during sequence analysis. In some embodiments, the UMI may provide function as a location identifier and also provide information in the macromolecule analysis assay. For example, the UMI may be used to identify molecules that are identical by descent, and therefore originated from the same initial molecule. In some aspects, this information can be used to correct for variations in amplification, and to detect and correct sequencing errors.

In some embodiments, the recording tag comprises a spacer polymer. In certain embodiments, a recording tag comprises a spacer at its terminus, e.g., 3′ end. As used herein reference to a spacer sequence in the context of a recording tag includes a spacer sequence that is identical to the spacer sequence associated with its cognate binding agent, or a spacer sequence that is complementary to the spacer sequence associated with its cognate binding agent. The terminal, e.g., 3′, spacer on the recording tag permits transfer of identifying information of a cognate binding agent from a secondary tag to the recording tag during the first binding cycle (e.g., via annealing of complementary spacer sequences for primer extension or sticky end ligation). In one embodiment, the spacer sequence is about 1-20 bases in length, about 2-12 bases in length, or 5-10 bases in length. The length of the spacer may depend on factors such as the temperature and reaction conditions of the primer extension reaction for transferring secondary tag information to the recording tag.

In some embodiments using spacer sequences, the recording tags associated with a library of polypeptides share a common spacer sequence. In other embodiments, the recording tags associated with a library of polypeptides have binding cycle specific spacer sequences that are complementary to the binding cycle specific spacer sequences of adaptor molecules. In some aspects, the spacer sequence in the recording tag is designed to have minimal complementarity to other regions in the recording tag; likewise, the spacer sequence in the adaptor molecules should have minimal complementarity to other regions in the adaptor molecule. In some cases, the spacer sequence of the recording tags and adaptor molecules should have minimal sequence complementarity to components such unique molecular identifiers, barcodes (e.g., compartment, partition, sample, spatial location), universal primer sequences, secondary tag sequences, cycle specific sequences, etc. present in the tags.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′-SEQ ID NO:1) or an Illumina P7 primer (5′-CAAGCAGAAGACGGCATACGAGAT-3′-SEQ ID NO:2).

In certain embodiments, a recording tag comprises a compartment tag. In some embodiments, the compartment tag is a component within a recording tag. In some embodiments, the recording tag can also include a barcode which represents a compartment tag in which a compartment, such as a droplet, microwell, physical region on a support, etc. is assigned a unique barcode. The association of a compartment with a specific barcode can be achieved in any number of ways such as by encapsulating a single barcoded bead in a compartment, e.g., by direct merging or adding a barcoded droplet to a compartment, by directly printing or injecting a barcode reagents to a compartment, etc. The barcode reagents within a compartment are used to add compartment-specific barcodes to the macromolecule or fragments thereof within the compartment. Applied to protein partitioning into compartments, the barcodes can be used to map analyzed peptides back to their originating protein molecules in the compartment. This can greatly facilitate protein identification. Compartment barcodes can also be used to identify protein complexes. In other embodiments, multiple compartments that represent a subset of a population of compartments may be assigned a unique barcode representing the subset. In some embodiments, the recording tag comprises fraction barcode which contains identifying information for the macromolecules within a fraction.

In some embodiments, one or more of the tags (e.g., compartment tag, a partition barcode, sample barcode, a fraction barcode, etc.) further comprise a functional moiety capable of reacting with an internal amino acid, the peptide backbone, or N-terminal amino acid on the plurality of protein complexes, proteins, or polypeptides. In some embodiments, the functional moiety is a click chemistry moiety, an aldehyde, an azide/alkyne, or a maleimide/thiol, or an epoxide/nucleophile, an inverse electron demand Diels-Alder (iEDDA) group, or a moiety for a Staudinger reaction. In some specific embodiments, a plurality of compartment tags is formed by printing, spotting, ink-jetting the compartment tags into the compartment, or a combination thereof. In some embodiments, the tag is attached to a polypeptide to link the tag to the macromolecule via a polypeptide-polypeptide linkage. In some embodiments, the tag-attached polypeptide comprises a protein ligase recognition sequence.

In certain embodiments, a peptide or polypeptide macromolecule can be immobilized to a support by an affinity capture reagent (and optionally covalently crosslinked), wherein the recording tag is associated with the affinity capture reagent directly, or alternatively, the macromolecule can be directly immobilized to the support with a recording tag. In one embodiment, the macromolecule is attached to a bait nucleic acid which hybridizes to a capture nucleic acid and is ligated to a capture nucleic acid which comprises a reactive coupling moiety for attaching to the support. In some examples, the bait or capture nucleic acid may serve as a recording tag to which information regarding the polypeptide can be transferred. In some embodiments, the macromolecule is attached to a bait nucleic acid to form a nucleic acid-macromolecule chimera. In some embodiments, the immobilization methods comprise bringing the nucleic acid-macromolecule chimera into proximity with a support by hybridizing the bait nucleic acid to a capture nucleic acid attached to the support, and covalently coupling the nucleic acid-macromolecule chimera to the solid support. In some cases, the nucleic acid-macromolecule chimera is coupled indirectly to the solid support, such as via a linker. In some embodiments, a plurality of the nucleic acid-macromolecule chimeras is coupled on the solid support and any adjacently coupled nucleic acid-macromolecule chimeras are spaced apart from each other at an average distance of about 50 nm or greater.

In some embodiments, the density or number of macromolecules provided with a recording tag is controlled or titrated. In some examples, the desired spacing, density, and/or amount of recording tags in the sample may be titrated by providing a diluted or controlled number of recording tags. In some examples, the desired spacing, density, and/or amount of recording tags may be achieved by spiking a competitor or “dummy” competitor molecule when providing, associating, and/or attaching the recording tags. In some cases, the “dummy” competitor molecule reacts in the same way as a recording tag being associated or attached to a macromolecule in the sample but the competitor molecule does not function as a recording tag. In some specific examples, if a desired density is 1 functional recording tag per 1,000 available sites for attachment in the sample, then spiking in 1 functional recording tag for every 1,000 “dummy” competitor molecules is used to achieve the desired spacing. In some examples, the ratio of functional recording tags is adjusted based on the reaction rate of the functional recording tags compared to the reaction rate of the competitor molecules.

In some examples, the labeling of the macromolecule with a recording tag is performed using standard amine coupling chemistries. For example, the e-amino group (e.g., of lysine residues) and the N-terminal amino group may be susceptible to labeling with amine-reactive coupling agents, depending on the pH of the reaction (Mendoza et al., Mass Spectrom Rev (2009) 28(5): 785-815). In a particular embodiment, the recording tag comprises a reactive moiety (e.g., for conjugation to a solid surface, a multifunctional linker, or a macromolecule), a linker, a universal priming sequence, a barcode (e.g., compartment tag, partition barcode, sample barcode, fraction barcode, or any combination thereof), an optional UMI, and a spacer (Sp) sequence for facilitating information transfer. In another embodiment, the protein can be first labeled with a universal DNA tag, and the barcode-Sp sequence (representing a sample, a compartment, a physical location on a slide, etc.) are attached to the protein later through and enzymatic or chemical coupling step. A universal DNA tag comprises a short sequence of nucleotides that are used to label a protein or polypeptide macromolecule and can be used as point of attachment for a barcode (e.g., compartment tag, recording tag, etc.). For example, a recording tag may comprise at its terminus a sequence complementary to the universal DNA tag. In certain embodiments, a universal DNA tag is a universal priming sequence. Upon hybridization of the universal DNA tags on the labeled protein to complementary sequence in recording tags (e.g., bound to beads), the annealed universal DNA tag may be extended via primer extension, transferring the recording tag information to the DNA tagged protein. In a particular embodiment, the protein is labeled with a universal DNA tag prior to proteinase digestion into peptides. The universal DNA tags on the labeled peptides from the digest can then be converted into an informative and effective recording tag.

The recording tags may comprise a reactive moiety for a cognate reactive moiety present on the macromolecule, e.g., protein, (e.g., click chemistry labeling, photoaffinity labeling). For example, recording tags may comprise an azide moiety for interacting with alkyne-derivatized proteins, or recording tags may comprise a benzophenone for interacting with native proteins, etc. Upon binding of the target protein by the target protein specific binding agent, the recording tag and target protein are coupled via their corresponding reactive moieties. After the target protein is labeled with the recording tag, the target-protein specific binding agent may be removed by digestion of the DNA capture probe linked to the target-protein specific binding agent. For example, the DNA capture probe may be designed to contain uracil bases, which are then targeted for digestion with a uracil-specific excision reagent (e.g., USER™), and the target-protein specific binding agent may be dissociated from the target protein. In some embodiments, other types of linkages besides hybridization can be used to link the recording tag to a macromolecule. A suitable linker can be attached to various positions of the recording tag, such as the 3′ end, at an internal position, or within the linker attached to the 5′ end of the recording tag.

In some aspects, the spacer sequence in the recording is designed to have minimal complementarity to other regions in the recording tag. In some aspects, the spacer sequence of the recording tags and adaptor molecules should have minimal sequence complementarity to components such unique molecular identifiers, barcodes (e.g., compartment, partition, sample, spatial location), universal primer sequences, secondary tag sequences, cycle specific sequences, etc. present in the recording tags, adaptor molecules, and/or coding tags.

The information from one or more secondary tags of adaptor molecules is transferred to the recording tag to generate an extended recording tag. In some embodiments, an extended recording tag comprises from 5′ to 3′ direction: a universal forward (or 5′) priming sequence, information transferred from one or more secondary tag(s), and a spacer sequence. In some embodiments, an extended recording tag comprises from 5′ to 3′ direction: a universal forward (or 5′) priming sequence, information transferred from one or more secondary tag(s), optionally other barcodes (e.g., sample barcode, partition barcode, compartment barcode, or any combination thereof), a spacer sequence, and a universal reverse (or 3′) priming sequence. In some other embodiments, a recording tag comprises from 5′ to 3′ direction: a universal forward (or 5′) priming sequence, information transferred from one or more secondary tag(s), optionally other barcodes (e.g., sample barcode, partition barcode, compartment barcode, or any combination thereof), an optional UMI, a spacer sequence, and a universal reverse (or 3′) priming sequence.

C. Binding Agent

The methods described herein use a binding agent configured for interacting with the macromolecules to be analyzed (e.g., polypeptides, peptides, proteins). The assay can include contacting a plurality of binding agents to a plurality of macromolecules. In some embodiments, the present methods comprise contacting a single macromolecule with a single binding agent, contacting a plurality of macromolecules with a single binding agent, or contacting a plurality of macromolecules with a plurality of binding agents. In some embodiments, the plurality of binding agents includes a mixture of binding agents.

A binding agent can be any molecule (e.g., peptide, polypeptide, protein, nucleic acid, carbohydrate, small molecule, and the like) capable of binding to a component or feature of a polypeptide. A binding agent can be a naturally occurring, synthetically produced, or recombinantly expressed molecule. In some embodiments, the scaffold used to engineer a binding agent can be from any species, e.g., human, non-human, transgenic. A binding agent may bind to a portion of a target macromolecule or a motif. A binding agent may bind to a single monomer or subunit of a polypeptide (e.g., a single amino acid) or bind to multiple linked subunits of a polypeptide (e.g., dipeptide, tripeptide, or higher order peptide of a longer polypeptide molecule).

In some examples, the binding agent comprises an antibody, an antigen-binding antibody fragment, a single-domain antibody (sdAb), a recombinant heavy-chain-only antibody (VHH), a single-chain antibody (scFv), a shark-derived variable domain (vNARs), a Fv, a Fab, a Fab′, a F(ab′)2, a linear antibody, a diabody, an aptamer, a peptide mimetic molecule, a fusion protein, a reactive or non-reactive small molecule, or a synthetic molecule.

In certain embodiments, a binding agent may be designed to bind covalently. Covalent binding can be designed to be conditional or favored upon binding to the correct moiety. For example, an target and its cognate binding agent may each be modified with a reactive group such that once the target-specific binding agent is bound to the target, a coupling reaction is carried out to create a covalent linkage between the two. Non-specific binding of the binding agent to other locations that lack the cognate reactive group would not result in covalent attachment. In some embodiments, the target comprises a ligand that is capable of forming a covalent bond to a binding agent. In some embodiments, the target comprises a ligand group that is capable of covalent binding to a binding agent. Covalent binding between a binding agent and its target may allow for more stringent washing to be used to remove binding agents that are non-specifically bound, thus increasing the specificity of the assay. In some embodiments, the method includes a wash step after contacting the binding agent to the macromolecule to remove non-specifically bound binding agents. The stringency of the wash step may be tuned depending on the affinity of the binding agent to the target and/or the strength and stability of the complex formed.

In some embodiments, the binding agents are configured to provide specificity for binding of the binding agent to the macromolecule. In certain embodiments, a binding agent may be a selective binding agent. As used herein, selective binding refers to the ability of the binding agent to preferentially bind to a specific ligand (e.g., amino acid or class of amino acids) relative to binding to a different ligand (e.g., amino acid or class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding, hydrophobic binding, and Van der Waals forces (non-covalent interactions) or by reversible or non-reversible covalent attachment to the binding agent. It should also be understood that selectivity may be relative, and as opposed to absolute, and that different factors can affect the same, including ligand concentration. Thus, in one example, a binding agent selectively binds one of the twenty standard amino acids. In some examples, a binding agent binds to an N-terminal amino acid residue, a C-terminal amino acid residue, or an internal amino acid residue.

In some embodiments, the binding agent is partially specific or selective. In some aspects, the binding agent preferentially binds one or more amino acids. In some examples, a binding agent may bind to or is capable of binding to two or more of the twenty standard amino acids. For example, a binding agent may preferentially bind the amino acids A, C, and G over other amino acids. In some other examples, the binding agent may selectively or specifically bind more than one amino acid. In some aspects, the binding agent may also have a preference for one or more amino acids at the second, third, fourth, fifth, etc. positions from the terminal amino acid. In some cases, the binding agent preferentially binds to a specific terminal amino acid and a penultimate amino acid. For example, a binding agent may preferentially bind AA, AC, and AG or a binding agent may preferentially bind AA, CA, and GA. In some specific examples, binding agents with different specificities can be associated with coding tags that hybridize to adaptor molecules comprising the same secondary tag. In some embodiments, a binding agent may exhibit flexibility and variability in target binding preference in some or all of the positions of the targets. In some examples, a binding agent may have a preference for one or more specific target terminal amino acids and have a flexible preference for a target at the penultimate position. In some other examples, a binding agent may have a preference for one or more specific target amino acids in the penultimate amino acid position and have a flexible preference for a target at the terminal amino acid position. In some embodiments, a binding agent is selective for a target comprising a terminal amino acid and other components of a macromolecule. In some examples, a binding agent is selective for a target comprising a terminal amino acid and at least a portion of the peptide backbone. In some particular examples, a binding agent is selective for a target comprising a terminal amino acid and an amide peptide backbone. In some cases, the peptide backbone comprises a natural peptide backbone or a post-translational modification. In some embodiments, the binding agent exhibits allosteric binding.

In some embodiments, the method comprises contacting a mixture of binding agents with a mixture of macromolecules and selectivity need only be relative to the other binding agents to which the target is exposed. It should also be understood that selectivity of a binding agent need not be absolute to a specific molecule but could be to a portion of a molecule. In some examples, selectivity of a binding agent need not be absolute to a specific amino acid, but could be selective to a class of amino acids, such as amino acids with polar or non-polar side chains, or with electrically (positively or negatively) charged side chains, or with aromatic side chains, or some specific class or size of side chains, and the like. In some embodiments, the ability of a binding agent to selectively bind a feature or component of a macromolecule is characterized by comparing binding abilities of binding agents. For example, the binding ability of a binding agent to the target can be compared to the binding ability of a binding agent which binds to a different target, for example, comparing a binding agent selective for a class of amino acids to a binding agent selective for a different class of amino acids. In some examples, a binding agent selective for non-polar side chains is compared to a binding agent selective for polar side chains. In some embodiments, a binding agent selective for a feature, component of a peptide, or one or more amino acid exhibits at least 1×, at least 2×, at least 5×, at least 10×, at least 50×, at least 100×, or at least 500× more binding compared to a binding agent selective for a different feature, component of a peptide, or one or more amino acid.

In a particular embodiment, the binding agent has a high affinity and high selectivity for the macromolecule, e.g., the polypeptide, of interest. In particular, a high binding affinity with a low off-rate may be efficacious for hybridization of the adaptor molecule to the coding tag. In certain embodiments, a binding agent has a Kd of about <500 nM, <200 nM, <100 nM, <50 nM, <10 nM, <5 nM, <1 nM, <0.5 nM, or <0.1 nM. In a particular embodiment, the binding agent is added to the polypeptide at a concentration >1×, >5×, >10×, >100×, or >1000× its Kd to drive binding to completion. For example, binding kinetics of an antibody to a single protein molecule is described in Chang et al., J Immunol Methods (2012) 378(1-2): 102-115.

In certain embodiments, a binding agent may bind to a terminal amino acid of a peptide, an intervening amino acid, dipeptide (sequence of two amino acids), tripeptide (sequence of three amino acids), or higher order peptide of a peptide molecule. In some embodiments, each binding agent in a library of binding agents selectively binds to a particular amino acid, for example one of the twenty standard naturally occurring amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the binding agent binds to an unmodified or native (e.g., natural) amino acid. In some examples, the binding agent binds to an unmodified or native dipeptide (sequence of two amino acids), tripeptide (sequence of three amino acids), or higher order peptide of a peptide molecule. A binding agent may be engineered for high affinity for a native or unmodified N-terminal amino acid (NTAA), high specificity for a native or unmodified NTAA, or both. In some embodiments, binding agents can be developed through directed evolution of promising affinity scaffolds using phage display.

In certain embodiments, a binding agent may bind to a post-translational modification of an amino acid. In some embodiments, a peptide comprises one or more post-translational modifications, which may be the same of different. The NTAA, CTAA, an intervening amino acid, or a combination thereof of a peptide may be post-translationally modified. Post-translational modifications to amino acids include acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation (see, also, Seo and Lee, 2004, J. Biochem. Mol. Biol. 37:35-44).

In certain embodiments, a lectin is used as a binding agent for detecting the glycosylation state of a protein, polypeptide, or peptide. Lectins are carbohydrate-binding proteins that can selectively recognize glycan epitopes of free carbohydrates or glycoproteins. A list of lectins recognizing various glycosylation states (e.g., core-fucose, sialic acids, N-acetyl-D-lactosamine, mannose, N-acetyl-glucosamine) include: A, AAA, AAL, ABA, ACA, ACG, ACL, AOL, ASA, BanLec, BC2L-A, BC2LCN, BPA, BPL, Calsepa, CGL2, CNL, Con, ConA, DBA, Discoidin, DSA, ECA, EEL, F17AG, Gal1, Gal1-S, Gal2, Gal3, Gal3C-S, Gal7-S, Gal9, GNA, GRFT, GS-I, GS-II, GSL-I, GSL-II, HHL, HIHA, HPA, I, II, Jacalin, LBA, LCA, LEA, LEL, Lentil, Lotus, LSL-N, LTL, MAA, MAH, MAL_I, Malectin, MOA, MPA, MPL, NPA, Orysata, PA-IIL, PA-IL, PALa, PHA-E, PHA-L, PHA-P, PHAE, PHAL, PNA, PPL, PSA, PSL1a, PTL, PTL-I, PWM, RCA120, RS-Fuc, SAMB, SBA, SJA, SNA, SNA-I, SNA-II, SSA, STL, TJA-I, TJA-II, TxLCI, UDA, UEA-I, UEA-II, VFA, VVA, WFA, WGA (see, Zhang et al., 2016, MABS 8:524-535).

In some embodiments, a binding agent may bind to a native or unmodified or unlabeled terminal amino acid. Moreover, in some cases, these natural amino acid binders don't recognize N-terminal labels. Directed evolution of aaRS scaffolds can be used to generate higher affinity, higher specificity binding agents that recognized the N-terminal amino acids in the context of an N-terminal label. In another example, Havranak et al. (U.S. Patent Publication No. US 2014/0273004) describes engineering aminoacyl tRNA synthetases (aaRSs) as specific NTAA binders. The amino acid binding pocket of the aaRSs has an intrinsic ability to bind cognate amino acids, but generally exhibits poor binding affinity and specificity. Moreover, these natural amino acid binders don't recognize N-terminal labels. Directed evolution of aaRS scaffolds can be used to generate higher affinity, higher specificity binding agents that recognized the N-terminal amino acids in the context of an N-terminal label.

In certain embodiments, a binding agent may bind to a modified or labeled terminal amino acid (e.g., an NTAA that has been functionalized or modified). In some embodiments, a binding agent may bind to a chemically or enzymatically modified terminal amino acid. A modified or labeled NTAA can be one that is functionalized with phenylisothiocyanate, PITC, 1-fluoro-2,4-dinitrobenzene (Sanger's reagent, DNFB), benzyloxycarbonyl chloride or carbobenzoxy chloride (Cbz-Cl), N-(Benzyloxycarbonyloxy)succinimide (Cbz-OSu or Cbz-O—NHS), dansyl chloride (DNS-Cl, or 1-dimethylaminonaphthalene-5-sulfonyl chloride), 4-sulfonyl-2-nitrofluorobenzene (SNFB), N-Acetyl-Isatoic Anhydride, Isatoic Anhydride, 2-Pyridinecarboxaldehyde, 2-Formylphenylboronic acid, 2-Acetylphenylboronic acid, 1-Fluoro-2,4-dinitrobenzene, Succinic anhydride, 4-Chloro-7-nitrobenzofurazan, Pentafluorophenylisothiocyanate, 4-(Trifluoromethoxy)-phenylisothiocyanate, 4-(Trifluoromethyl)-phenylisothiocyanate, 3-(Carboxylic acid)-phenylisothiocyanate, 3-(Trifluoromethyl)-phenylisothiocyanate, 1-Naphthylisothiocyanate, N-nitroimidazole-1-carboximidamide, N,N,Ä≤-Bis(pivaloyl)-1H-pyrazole-1-carboxamidine, N,N,Ä≤-Bis(benzyloxycarbonyl)-1H-pyrazole-1-carboxamidine, an acetylating reagent, a guanidinylation reagent, a thioacylation reagent, a thioacetylation reagent, or a thiobenzylation reagent, or a diheterocyclic methanimine reagent. In some examples, the binding agent binds an amino acid labeled by contacting with a reagent or using a method as described in International Patent Publication No. WO 2019/089846. In some cases, the binding agent binds an amino acid labeled by an amine modifying reagent.

A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may bind to an N-terminal or C-terminal diamino acid moiety. An N-terminal diamino acid is comprised of the N-terminal amino acid and the penultimate N-terminal amino acid. A C-terminal diamino acid is similarly defined for the C-terminus. In some embodiments, the binding agent binds to a chemically modified N-terminal amino acid residue or a chemically modified C-terminal amino acid residue. To increase the affinity of a binding agent to small N-terminal amino acids (NTAAs) of peptides, the NTAA may be modified with an “immunogenic” hapten, such as dinitrophenol (DNP). This can be implemented in a cyclic sequencing approach using Sanger's reagent, dinitrofluorobenzene (DNFB), which attaches a DNP group to the amine group of the NTAA. Commercial anti-DNP antibodies have affinities in the low nM range (˜8 nM, LO-DNP-2) (Bilgicer et al., J Am Chem Soc (2009) 131(26): 9361-9367); as such it stands to reason that it should be possible to engineer high-affinity NTAA binding agents to a number of NTAAs modified with DNP (via DNFB) and simultaneously achieve good binding selectivity for a particular NTAA. In another example, an NTAA may be modified with sulfonyl nitrophenol (SNP) using 4-sulfonyl-2-nitrofluorobenzene (SNFB). Similar affinity enhancements may also be achieved with alternative NTAA modifiers, such as an acetyl group or an amidinyl (guanidinyl) group.

In certain embodiments, a binding agent can be an aptamer (e.g., peptide aptamer, DNA aptamer, or RNA aptamer), a peptoid, an antibody or a specific binding fragment thereof, an amino acid binding protein or enzyme, an antibody binding fragment, an antibody mimetic, a peptide, a peptidomimetic, a protein, or a polynucleotide (e.g., DNA, RNA, peptide nucleic acid (PNA), a gPNA, bridged nucleic acid (BNA), xeno nucleic acid (XNA), glycerol nucleic acid (GNA), or threose nucleic acid (TNA), or a variant thereof).

As used herein, the terms antibody and antibodies are used in a broad sense, to include not only intact antibody molecules, for example but not limited to immunoglobulin A, immunoglobulin G, immunoglobulin D, immunoglobulin E, and immunoglobulin M, but also any immunoreactive component(s) of an antibody molecule or portion thereof that immuno-specifically bind to at least one epitope. An antibody may be naturally occurring, synthetically produced, or recombinantly expressed. An antibody may be a fusion protein. An antibody may be an antibody mimetic. Examples of antibodies include but are not limited to, Fab fragments, Fab′ fragments, F(ab)₂ fragments, single chain antibody fragments (scFv), miniantibodies, nanobodies, diabodies, crosslinked antibody fragments, Affibody™, nanobodies, single domain antibodies, DVD-Ig molecules, alphabodies, affimers, affitins, cyclotides, molecules, and the like. Immunoreactive products derived using antibody engineering or protein engineering techniques are also expressly within the meaning of the term antibodies. Detailed descriptions of antibody and/or protein engineering, including relevant protocols, can be found in, among other places, J. Maynard and G. Georgiou, 2000, Ann. Rev. Biomed. Eng. 2:339-76; Antibody Engineering, R. Kontermann and S. Dubel, eds., Springer Lab Manual, Springer Verlag (2001); U.S. Pat. No. 5,831,012; and S. Paul, Antibody Engineering Protocols, Humana Press (1995).

As with antibodies, nucleic acid and peptide aptamers that specifically recognize a macromolecule, e.g., a peptide or a polypeptide, can be produced using known methods. Aptamers bind target molecules in a highly specific, conformation-dependent manner, typically with very high affinity, although aptamers with lower binding affinity can be selected if desired. Aptamers have been shown to distinguish between targets based on very small structural differences such as the presence or absence of a methyl or hydroxyl group and certain aptamers can distinguish between D- and L-enantiomers. Aptamers have been obtained that bind small molecular targets, including drugs, metal ions, and organic dyes, peptides, biotin, and proteins, including but not limited to streptavidin, VEGF, and viral proteins. Aptamers have been shown to retain functional activity after biotinylation, fluorescein labeling, and when attached to glass surfaces and microspheres. (see, e.g., Jayasena, 1999, Clin Chem 45:1628-50; Kusser2000, J. Biotechnol. 74: 27-39; Colas, 2000, Curr Opin Chem Biol 4:54-9). Aptamers which specifically bind arginine and AMP have been described as well (see, Patel and Suri, 2000, J. Biotech. 74:39-60). Oligonucleotide aptamers that bind to a specific amino acid have been disclosed in Gold et al. (1995, Ann. Rev. Biochem. 64:763-97). RNA aptamers that bind amino acids have also been described (Ames and Breaker, 2011, RNA Biol. 8; 82-89; Mannironi et al., 2000, RNA 6:520-27; Famulok, 1994, J. Am. Chem. Soc. 116:1698-1706).

A binding agent can be made by modifying naturally-occurring or synthetically-produced proteins by genetic engineering to introduce one or more mutations in the amino acid sequence to produce engineered proteins that bind to a specific component or feature of a polypeptide (e.g., NTAA, CTAA, or post-translationally modified amino acid or a peptide). For example, exopeptidases (e.g., aminopeptidases, carboxypeptidases, dipeptidyl peptidase, dipeptidyl aminopeptidase), exoproteases, mutated exoproteases, mutated anticalins, mutated ClpSs, antibodies, or tRNA synthetases can be modified to create a binding agent that selectively binds to a particular NTAA. In another example, carboxypeptidases can be modified to create a binding agent that selectively binds to a particular CTAA. A binding agent can also be designed or modified, and utilized, to specifically bind a modified NTAA or modified CTAA, for example one that has a post-translational modification (e.g., phosphorylated NTAA or phosphorylated CTAA) or one that has been modified with a label (e.g., PTC, 1-fluoro-2,4-dinitrobenzene (using Sanger's reagent, DNFB), dansyl chloride (using DNS-Cl, or 1-dimethylaminonaphthalene-5-sulfonyl chloride), or using a thioacylation reagent, a thioacetylation reagent, an acetylation reagent, an amidination (guanidinylation) reagent, or a thiobenzylation reagent). Strategies for directed evolution of proteins are known in the art (e.g., Yuan et al., 2005, Microbiol. Mol. Biol. Rev. 69:373-392), and include phage display, ribosomal display, mRNA display, CIS display, CAD display, emulsions, cell surface display method, yeast surface display, bacterial surface display, etc.

In some embodiments, a binding agent that selectively binds to a labeled or functionalized NTAA can be utilized. For example, the NTAA may be reacted with phenylisothiocyanate (PITC) to form a phenylthiocarbamoyl-NTAA derivative. In this manner, the binding agent may be fashioned to selectively bind both the phenyl group of the phenylthiocarbamoyl moiety as well as the alpha-carbon R group of the NTAA. Use of PITC in this manner allows for subsequent elimination of the NTAA by Edman degradation as discussed below. In another embodiment, the NTAA may be reacted with Sanger's reagent (DNFB), to generate a DNP-labeled NTAA. Optionally, DNFB is used with an ionic liquid such as 1-ethyl-3-methylimidazolium bis[(trifluoromethyl)sulfonyl]imide ([emim][Tf2N]), in which DNFB is highly soluble. In this manner, the binding agent may be engineered to selectively bind the combination of the DNP and the R group on the NTAA. The addition of the DNP moiety provides a larger “handle” for the interaction of the binding agent with the NTAA, and should lead to a higher affinity interaction.

In yet another embodiment, a binding agent may be a modified aminopeptidase. In some embodiments, the binding agent may be a modified aminopeptidase that has been engineered to recognize the DNP-labeled NTAA providing cyclic control of aminopeptidase degradation of the peptide. Once the DNP-labeled NTAA is eliminated, another cycle of DNFB derivatization is performed in order to bind and eliminate the newly exposed NTAA. In preferred particular embodiment, the aminopeptidase is a monomeric metallo-protease, such an aminopeptidase activated by zinc (Calcagno et al., Appl Microbiol Biotechnol. (2016) 100(16):7091-7102). In another example, a binding agent may selectively bind to an NTAA that is modified with sulfonyl nitrophenol (SNP), e.g., by using 4-sulfonyl-2-nitrofluorobenzene (SNFB). Other reagents that may be used to functionalize the NTAA include trifluoroethyl isothiocyanate, allyl isothiocyanate, and dimethylaminoazobenzene isothiocyanate, or a reagent as described in International Patent Publication No. WO 2019/089846.

In some embodiments, the binding agent binds to an unmodified or native amino acid. In some examples, the binding agent binds to an unmodified or native dipeptide (sequence of two amino acids), tripeptide (sequence of three amino acids), or higher order peptide of a peptide molecule. A binding agent may be engineered for high affinity for a modified NTAA, high specificity for a modified NTAA, or both. In some embodiments, binding agents can be developed through directed evolution of promising affinity scaffolds using phage display.

In another example, highly-selective engineered ClpSs have also been described in the literature. Emili et al. describe the directed evolution of an E. coli. ClpS protein via phage display, resulting in four different variants with the ability to selectively bind NTAAs for aspartic acid, arginine, tryptophan, and leucine residues (U.S. Pat. No. 9,566,335, incorporated by reference in its entirety). In one embodiment, the binding moiety of the binding agent comprises a member of the evolutionarily conserved ClpS family of adaptor proteins involved in natural N-terminal protein recognition and binding or a variant thereof. (See e.g., Schuenemann et al., (2009) EMBO Reports 10(5); Roman-Hernandez et al., (2009) PNAS 106(22):8888-93; Guo et al., (2002) JBC 277(48): 46753-62; Wang et al., (2008) Molecular Cell 32: 406-414). In some embodiments, the amino acid residues corresponding to the ClpS hydrophobic binding pocket identified in Schuenemann et al. are modified in order to generate a binding moiety with the desired selectivity.

In one embodiment, the binding moiety comprises a member of the UBR box recognition sequence family, or a variant of the UBR box recognition sequence family. UBR recognition boxes are described in Tasaki et al., (2009), JBC 284(3): 1884-95. For example, the binding moiety may comprise UBR1, UBR2, or a mutant, variant, or homologue thereof.

In certain embodiments, the binding agent further comprises one or more detectable labels such as fluorescent labels, in addition to the binding moiety. In some embodiments, the binding agent does not comprise a polynucleotide such as a coding tag. Optionally, the binding agent comprises a synthetic or natural antibody. In some embodiments, the binding agent comprises an aptamer. In one embodiment, the binding agent comprises a polypeptide, such as a modified member of the ClpS family of adaptor proteins, such as a variant of an E. coli ClpS binding polypeptide, and a detectable label. In one embodiment, the detectable label is optically detectable. In some embodiments, the detectable label comprises a fluorescently moiety, a color-coded nanoparticle, a quantum dot or any combination thereof. In one embodiment the label comprises a polystyrene dye encompassing a core dye molecule such as a FluoSphere™, Nile Red, fluorescein, rhodamine, derivatized rhodamine dyes, such as TAMRA, phosphor, polymethadine dye, fluorescent phosphoramidite, TEXAS RED, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA or a derivative or modification of any of the foregoing. In one embodiment, the detectable label is resistant to photobleaching while producing lots of signal (such as photons) at a unique and easily detectable wavelength, with high signal-to-noise ratio.

In a particular embodiment, anticalins are engineered for both high affinity and high specificity to labeled NTAAs (e.g. PTC, modified-PTC, Cbz, DNP, SNP, acetyl, guanidinyl, amino guanidinyl, heterocyclic methanimine, etc.). Certain varieties of anticalin scaffolds have suitable shape for binding single amino acids, by virtue of their beta barrel structure. An N-terminal amino acid (either with or without modification) can potentially fit and be recognized in this “beta barrel” bucket. High affinity anticalins with engineered novel binding activities have been described (reviewed by Skerra, 2008, FEBS J. 275: 2677-2683). For example, anticalins with high affinity binding (low nM) to fluorescein and digoxygenin have been engineered (Gebauer et al., 2012, Methods Enzymol 503: 157-188.). Engineering of alternative scaffolds for new binding functions has also been reviewed by Banta et al. (2013, Annu. Rev. Biomed. Eng. 15:93-113).

The functional affinity (avidity) of a given monovalent binding agent may be increased by at least an order of magnitude by using a bivalent or higher order multimer of the monovalent binding agent (Vauquelin et al., 2013, Br J Pharmacol 168(8): 1771-1785. 2013). Avidity refers to the accumulated strength of multiple, simultaneous, non-covalent binding interactions. An individual binding interaction may be easily dissociated. However, when multiple binding interactions are present at the same time, transient dissociation of a single binding interaction does not allow the binding protein to diffuse away and the binding interaction is likely to be restored.

In some embodiments, the binding agent is linked, directly or indirectly, to a multimerization domain. Thus, monomeric, dimeric, and higher order (e.g., 3, 4, 5, or more) multimeric polypeptides comprising one or more binding agents are provided herein. In some specific embodiments, the binding agent is dimeric. In some examples, two polypeptides of the invention can be covalently or non-covalently attached to each other to form a dimer.

In some embodiments, the binding agent is derived from a biological, naturally occurring, non-naturally occurring, or synthetic source. In some examples, the binding agent is derived from de novo protein design (Huang et al., (2016) 537(7620):320-327). In some examples, the binding agent has a structure, sequence, and/or activity designed from first principles.

In some embodiments, a binding agent can be utilized that selectively binds a modified C-terminal amino acid (CTAA). Carboxypeptidases are proteases that cleave/eliminate terminal amino acids containing a free carboxyl group. A number of carboxypeptidases exhibit amino acid preferences, e.g., carboxypeptidase B preferentially cleaves at basic amino acids, such as arginine and lysine. A carboxypeptidase can be modified to create a binding agent that selectively binds to particular amino acid. In some embodiments, the carboxypeptidase may be engineered to selectively bind both the modification moiety as well as the alpha-carbon R group of the CTAA. Thus, engineered carboxypeptidases may specifically recognize 20 different CTAAs representing the standard amino acids in the context of a C-terminal label. Control of the stepwise degradation from the C-terminus of the peptide is achieved by using engineered carboxypeptidases that are only active (e.g., binding activity or catalytic activity) in the presence of the label. In one example, the CTAA may be modified by a para-Nitroanilide or 7-amino-4-methylcoumarinyl group.

Other potential scaffolds that can be engineered to generate binding agents for use in the methods described herein include: an anticalin, a lipocalin, an amino acid tRNA synthetase (aaRS), ClpS, an Affilin®, an Adnectin™, a T cell receptor, a zinc finger protein, a thioredoxin, GST A1-1, DARPin, an affimer, an affitin, an alphabody, an avimer, a monobody, an antibody, a single domain antibody, a nanobody, EETI-II, HPSTI, intrabody, PHD-finger, V(NAR) LDTI, evibody, Ig(NAR), knottin, maxibody, microbody, neocarzinostatin, pVIII, tendamistat, VLR, protein A scaffold, MTI-II, ecotin, GCN4, Im9, kunitz domain, PBP, trans-body, tetranectin, WW domain, CBM4-2, DX-88, GFP, iMab, Ldl receptor domain A, Min-23, PDZ-domain, avian pancreatic polypeptide, charybdotoxin/10Fn3, domain antibody (Dab), a2p8 ankyrin repeat, insect defensing A peptide, Designed AR protein, C-type lectin domain, staphylococcal nuclease, Src homology domain 3 (SH3), or Src homology domain 2 (SH2). See e.g., El-Gebali et al., (2019) Nucleic Acids Research 47:D427-D432 and Finn et al., (2013) Nucleic Acids Res. 42(Database issue):D222-D230. In some embodiments, a binding agent is derived from an enzyme which binds one or more amino acids (e.g., an aminopeptidase). In certain embodiments, a binding agent can be derived from an anticalin or a Clp protease adaptor protein (ClpS).

A binding agent may preferably bind to a modified or labeled amino acid, by chemical or enzymatic means, (e.g., an amino acid that has been functionalized by a reagent (e.g., a compound)) over a non-modified or unlabeled amino acid. For example, a binding agent may preferably bind to an amino acid that has been functionalized with an acetyl moiety, Cbz moiety, guanyl moiety, dansyl moiety, PTC moiety, DNP moiety, SNP moiety, dihaerocyclic methanimine moiety, etc., over an amino acid that does not possess said moiety. In some embodiments, a binding agent may preferably bind to an amino acid that has been functionalized or modified as described in International Patent Publication No. WO 2019/089846. The N-terminal amino acid (or labeled N-terminal amino acid, e.g., PITC-NTAA, Cbz-NTAA, DNP-NTAA, SNP-NTAA, acetyl-NTAA, guanidinylated-NTAA, heterocyclic methanimine-NTAA) of each immobilized peptide is bound by a cognate NTAA binding agent which is attached to a coding tag.

In some cases, a binding agent may bind to a post-translationally modified amino acid. In some embodiments, detection of internal post-translationally modified amino acids (e.g., phosphorylation, glycosylation, succinylation, ubiquitination, S-Nitrosylation, methylation, N-acetylation, lipidation, etc.) is be accomplished prior to detection and elimination of terminal amino acids (e.g., NTAA or CTAA). In one example, a peptide is contacted with binding agents for PTM modifications, and information from a corresponding secondary tag is transferred to the recording tag associated with the immobilized peptide. Once the detection and transfer of information relating to amino acid modifications is complete, the PTM modifying groups can be removed before detection and transfer of secondary tag information for the primary amino acid sequence using N-terminal or C-terminal degradation methods. Thus, resulting extended nucleic acids indicate the presence of post-translational modifications in a peptide sequence, though not the sequential order, along with primary amino acid sequence information.

In some embodiments, detection of internal post-translationally modified amino acids may occur concurrently with detection of primary amino acid sequence. In one example, an NTAA (or CTAA) is contacted with a binding agent specific for a post-translationally modified amino acid, either alone or as part of a library of binding agents (e.g., library composed of binding agents for the 20 standard amino acids and selected post-translational modified amino acids). Successive cycles of terminal amino acid elimination and contact with a binding agent (or library of binding agents) follow. Thus, resulting extended nucleic acids on the recording tag associated with the immobilized peptide indicate the presence and order of post-translational modifications in the context of a primary amino acid sequence.

In certain embodiments, a macromolecule, e.g., a polypeptide, is also contacted with a non-cognate binding agent. As used herein, a non-cognate binding agent is referring to a binding agent that is selective for a different target (e.g. polypeptide feature or component) than the particular target being considered. For example, if the n NTAA is phenylalanine, and the peptide is contacted with three binding agents selective for phenylalanine, tyrosine, and asparagine, respectively, the binding agent selective for phenylalanine would be first binding agent capable of selectively binding to the n^{th} NTAA (i.e., phenylalanine), while the other two binding agents would be non-cognate binding agents for that peptide (since they are selective for NTAAs other than phenylalanine). The tyrosine and asparagine binding agents may, however, be cognate binding agents for other peptides in the sample. If the n NTAA (phenylalanine) was then cleaved from the peptide, thereby converting the n−1 amino acid of the peptide to the n−1 NTAA (e.g., tyrosine), and the peptide was then contacted with the same three binding agents, the binding agent selective for tyrosine would be second binding agent capable of selectively binding to the n−1 NTAA (i.e., tyrosine), while the other two binding agents would be non-cognate binding agents (since they are selective for NTAAs other than tyrosine).

Thus, it should be understood that whether an agent is a binding agent or a non-cognate binding agent will depend on the nature of the particular polypeptide feature or component currently available for binding. Also, if multiple polypeptides are analyzed in a multiplexed reaction, a binding agent for one polypeptide may be a non-cognate binding agent for another, and vice versa. According, it should be understood that the following description concerning binding agents is applicable to any type of binding agent described herein (i.e., both cognate and non-cognate binding agents).

In certain embodiments, the concentration of the binding agents in a solution is controlled to reduce background and/or false positive results of the assay.

In some embodiments, the concentration of a binding agent can be at any suitable concentration, e.g., at about 0.0001 nM, about 0.001 nM, about 0.01 nM, about 0.1 nM, about 1 nM, about 2 nM, about 5 nM, about 10 nM, about 20 nM, about 50 nM, about 100 nM, about 200 nM, about 500 nM, or about 1,000 nM. In other embodiments, the concentration of a soluble conjugate used in the assay is between about 0.0001 nM and about 0.001 nM, between about 0.001 nM and about 0.01 nM, between about 0.01 nM and about 0.1 nM, between about 0.1 nM and about 1 nM, between about 1 nM and about 2 nM, between about 2 nM and about 5 nM, between about 5 nM and about 10 nM, between about 10 nM and about 20 nM, between about 20 nM and about 50 nM, between about 50 nM and about 100 nM, between about 100 nM and about 200 nM, between about 200 nM and about 500 nM, between about 500 nM and about 1000 nM, or more than about 1,000 nM.

In some embodiments, the ratio between the soluble binding agent molecules and the immobilized macromolecule, e.g., polypeptides, can be at any suitable range, e.g., at about 0.00001:1, about 0.0001:1, about 0.001:1, about 0.01:1, about 0.1:1, about 1:1, about 2:1, about 5:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 10⁴:1, about 10⁵:1, about 10⁶:1, or higher, or any ratio in between the above listed ratios. Higher ratios between the soluble binding agent molecules and the immobilized polypeptide(s) and/or the nucleic acids can be used to drive the binding and/or the secondary tag information transfer to completion. This may be particularly useful for detecting and/or analyzing low abundance polypeptides in a sample.

D. Coding Tag

The coding tag associated with the binding agent is or comprises a polynucleotide with any suitable length, e.g., a nucleic acid molecule of about 2 bases to about 100 bases, including any integer including 2 and 100 and in between, that comprises identifying information for its associated binding agent. A coding tag comprises a sequence for hybridizing to a first hybridization sequence of an adaptor molecule. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety). A coding tag may comprise an encoder sequence or a sequence with identifying information. The coding tag or portion thereof may comprise a sequence substantially complementary or complementary to a first hybridization sequence of an adaptor molecule, thus the coding tag can be associated with the secondary tag which contains identifying information regarding the binding agent. The first hybridization sequence on an adaptor molecule comprises a substantially complementary or complementary sequence configured to hybridize to the coding tag (or portion thereof) directly attached to a binding agent. In some aspects, the coding tag is used in the provided methods to hybridize to said first hybridization on the adaptor molecule and localize the appropriate adaptor molecule for information transfer.

A coding tag may be a single stranded molecule, a double stranded molecule, or a partially double stranded. A coding tag may comprise blunt ends, overhanging ends, or one of each. In some embodiments, a coding tag is partially double stranded. In some embodiments, the coding tag may comprise a hairpin. In certain embodiments, the hairpin comprises mutually complementary nucleic acid regions are connected through a nucleic acid strand. In some embodiments, the nucleic acid hairpin can also further comprise 3′ and/or 5′ single-stranded region(s) extending from the double-stranded stem segment. In some examples, the hairpin comprises a single strand of nucleic acid.

In some embodiments, a binding agent described comprises a coding tag containing identifying information regarding (e.g., representing or correlating to) the binding agent. In some embodiments, the identifying information from the secondary tag corresponds to the coding tag and comprises information regarding the identity of the target bound by the binding agent. In some embodiments, the identifying information from the coding tag comprises or is associated with information regarding the identity of the one or more amino acid(s) on the peptide bound by the binding agent.

A coding tag is a nucleic acid molecule of about 3 bases to about 100 bases that provides unique identifying information for its associated binding agent. A coding tag may comprise about 3 to about 90 bases, about 3 to about 80 bases, about 3 to about 70 bases, about 3 to about 60 bases, about 3 bases to about 50 bases, about 3 bases to about 40 bases, about 3 bases to about 30 bases, about 3 bases to about 20 bases, about 3 bases to about 10 bases, or about 3 bases to about 8 bases. In some embodiments, a coding tag is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, 40 bases, 55 bases, 60 bases, 65 bases, 70 bases, 75 bases, 80 bases, 85 bases, 90 bases, 95 bases, or 100 bases in length. A coding tag may be composed of DNA, RNA, polynucleotide analogs, or a combination thereof. Polynucleotide analogs include PNA, gPNA, BNA, GNA, TNA, LNA, morpholino polynucleotides, 2′-O-Methyl polynucleotides, alkyl ribosyl substituted polynucleotides, phosphorothioate polynucleotides, and 7-deaza purine analogs.

A coding tag can be joined to a binding agent directly or indirectly, by any means known in the art, including covalent and non-covalent interactions. In some embodiments, a coding tag may be joined to binding agent enzymatically or chemically. In some embodiments, a coding tag may be joined to a binding agent via ligation. In other embodiments, a coding tag is joined to a binding agent via affinity binding pairs (e.g., biotin and streptavidin). In some cases, a coding tag may be joined to a binding agent to an unnatural amino acid, such as via a covalent interaction with an unnatural amino acid.

In some embodiments, a binding agent is joined to a coding tag via SpyCatcher-SpyTag interaction. The SpyTag peptide forms an irreversible covalent bond to the SpyCatcher protein via a spontaneous isopeptide linkage, thereby offering a genetically encoded way to create peptide interactions that resist force and harsh conditions (Zakeri et al., 2012, Proc. Natl. Acad. Sci. 109:E690-697; Li et al., 2014, J. Mol. Biol. 426:309-317). A binding agent may be expressed as a fusion protein comprising the SpyCatcher protein. In some embodiments, the SpyCatcher protein is appended on the N-terminus or C-terminus of the binding agent. The SpyTag peptide can be coupled to the coding tag using standard conjugation chemistries (Hermanson, Bioconjugate Techniques, (2013) Academic Press).

In some embodiments, an enzyme-based strategy is used to join the binding agent to a coding tag. For example, the binding agent may be joined to a coding tag using a formylglycine (FGly)-generating enzyme (FGE). In one example, a protein, e.g., SpyLigase, is used to join the binding agent to the coding tag (Fierer et al., Proc Natl Acad Sci USA. 2014; 111(13): E1176-E1181).

In other embodiments, a binding agent is joined to a coding tag via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A binding agent may be expressed as a fusion protein comprising the SnoopCatcher protein. In some embodiments, the SnoopCatcher protein is appended on the N-terminus or C-terminus of the binding agent. The SnoopTag peptide can be coupled to the coding tag using standard conjugation chemistries.

In yet other embodiments, a binding agent is joined to a coding tag via the HaloTag® protein fusion tag and its chemical ligand. HaloTag is a modified haloalkane dehalogenase designed to covalently bind to synthetic ligands (HaloTag ligands) (Los et al., 2008, ACS Chem. Biol. 3:373-382). The synthetic ligands comprise a chloroalkane linker attached to a variety of useful molecules. A covalent bond forms between the HaloTag and the chloroalkane linker that is highly specific, occurs rapidly under physiological conditions, and is essentially irreversible.

In some cases, a binding agent is joined to a coding tag by attaching (conjugating) using an enzyme, such as sortase-mediated labeling (See e.g., Antos et al., Curr Protoc Protein Sci. (2009) CHAPTER 15: Unit-15.3; International Patent Publication No. WO2013003555). The sortase enzyme catalyzes a transpeptidation reaction (See e.g., Falck et al, Antibodies (2018) 7(4):1-19). In some aspects, the binding agent is modified with or attached to one or more N-terminal or C-terminal glycine residues.

In some embodiments, a binding agent is joined to a coding tag using a cysteine bioconjugation method. In some embodiments, a binding agent is joined to a coding tag using π-clamp-mediated cysteine bioconjugation (See e.g., Zhang et al., Nat Chem. (2016) 8(2):120-128). In some cases, a binding agent is joined to a coding tag using 3-arylpropiolonitriles (APN)-mediated tagging (e.g. Koniev et al., Bioconjug Chem. 2014; 25(2):202-206).

### II. MACROMOLECULE ANALYSIS ASSAY

The provided methods for analysis of macromolecules, e.g., peptides, polypeptides, and proteins, which includes a step of transferring information to a recording tag may include additional steps, treatments, and reactions. In some embodiments, the macromolecule analysis assay is a next generation protein assay (NGPA) using multiple binding agents and enzymatically-mediated sequential information transfer. In some cases, the analysis assay is performed on immobilized target molecules bound a cognate binding agent (e.g., antibody) and forming a stable complex, then hybridizing an adaptor molecule comprising a sequence that is complementary to the coding tag and a secondary tag, and transferring information from the secondary tags of bound antibodies to the recording tag associated with the macromolecule. In some cases, the analysis assay is performed on immobilized macromolecules bound by two or more cognate binding agents (e.g., antibodies). After a cognate antibody binding event, an adaptor molecule hybridizes, and a combined primer extension and DNA nicking step is used to transfer information from the secondary tags of the adaptor molecules to the recording tag. In some cases, polyclonal antibodies (or mixed population of monoclonal antibody) to multivalent epitopes on a protein can be used for the assay. See e.g., International Patent Publication No. WO 2017/192633.

In some embodiments, the macromolecule is a polypeptide and a polypeptide analysis assay is performed. In some embodiments, the sequence (or a portion of the sequence thereof) and/or the identity of a target protein is determined using a polypeptide analysis assay. In some examples, the polypeptide analysis assay includes assessing at least a partial sequence or identity of the polypeptide using suitable techniques or procedures. For example, at least a partial sequence of the polypeptide can be assessed by N-terminal amino acid analysis or C-terminal amino acid analysis. In some embodiments, at least a partial sequence of the polypeptide can be assessed using a ProteoCode assay. In some examples, at least a partial sequence of the polypeptide can be assessed by the techniques or procedures disclosed and/or claimed in U.S. Provisional Patent Application Nos. 62/330,841, 62/339,071, 62/376,886, 62/579,844, 62/582,312, 62/583,448, 62/579,870, 62/579,840, and 62/582,916, and International Patent Publication Nos. WO 2017/192633, and WO/2019/089836, and WO 2019/089851.

In embodiments relating to methods of analyzing peptides or polypeptides, the method generally includes contacting a binding agent to terminal amino acid (e.g., NTAA or CTAA) of a polypeptide, protein or peptide, providing an adaptor molecule and allowing hybridization of complementary sequences, and transferring the information from the secondary tag to the recording tag associated with the polypeptide, protein or peptide, thereby generating a first order extended recording tag (see FIG. 1A-1D and FIG. 2A-2D). In some further embodiment, the method comprises labeling or modifying the macromolecule (e.g. peptide) prior to or after the polypeptide, protein or peptide is contacted with the binding agent. For example, the terminal amino acid of the polypeptide, protein or peptide bound by the binding agent may be a chemically labeled or modified terminal amino acid. In some further embodiments, the method further includes removing or eliminating the terminal amino acid (e.g., NTAA or CTAA) from the polypeptide, protein or peptide after the information transfer step. The terminal amino acid eliminated may be a chemically labeled or modified terminal amino acid. Removal of the NTAA by contacting with an enzyme or chemical reagents converts the penultimate amino acid of the polypeptide, protein or peptide to a terminal amino acid. The polypeptide analysis may include one or more cycles of binding with additional binding agents to the terminal amino acid, providing a plurality of adaptor molecules and allow hybridization between the coding tag and first hybridization sequence of the adaptor molecule, and transferring information from the secondary tags to the extended nucleic acid thereby generating a higher order extended recording tag containing information regarding two or more binding agents, and eliminating the terminal amino acid in a cyclic manner. Additional binding, hybridizing, transferring information, and removal, can occur as described above up to n amino acids to generate an n^{th} order extended nucleic acid, which collectively represent the polypeptide, protein or peptide. In some of any provided embodiments, steps including the NTAA in the described exemplary approach can be performed instead with a C terminal amino acid (CTAA).

In some embodiments, the order of the steps in the process for a degradation-based peptide or polypeptide sequencing assay can be reversed or be performed in various orders. For example, in some embodiments, the terminal amino acid labeling can be conducted before and/or after the polypeptide is bound to the binding agent.

In some embodiments, the polypeptide analysis assay includes performing an assay which utilizes the recording tag associated with the macromolecule, e.g., the polypeptide. The recording tag is used to record information gathered from one or more binding events between a binding agent and the macromolecule to be analyzed.

Provided herein is a method for analyzing a macromolecule comprising the steps of: (a) providing a macromolecule and an associated recording tag joined to a support; (b) contacting the macromolecule with a binding agent capable of binding to the macromolecule, wherein the binding agent comprises a coding tag with identifying information regarding the binding agent, to allow binding between the macromolecule and the binding agent; (c) providing an adaptor molecule comprising a first hybridization sequence substantially complementary or complementary to at least a portion of the coding tag, and a secondary tag, to allow hybridization between the adaptor molecule (or the first hybridization sequence) and the coding tag (or the portion of the coding tag); (d) transferring the information of the secondary tag to the recording tag to generate an extended recording tag; and analyzing the extended recording tag. In some cases, the binding agent is removed after step (d). In some embodiments, the method further includes adding a universal priming site to the extended recording tag, prior to analyzing the extended recording tag.

In some examples, step (a) is performed before steps (b), (c), and (d). In some embodiments, step (b) is performed before step (c) and step (d). In some cases, step (c) is performed before step (d). In some particular embodiments, the steps are performed in the order: (a), (b), (c), and (d), optionally repeating steps (b), (c), and (d) one or more times.

In some embodiments, the method is performed with one or more macromolecules and the method further includes step (e) removing the terminal amino acid (e.g., N-terminal amino acid (NTAA)) of the polypeptide, protein or peptide to expose a new terminal amino acid of the polypeptide, protein or peptide. In some cases, a cycle of steps (b), (c), (d) and (e) is repeated one or more times prior to analyzing the extended recording tag. In some particular embodiments, the steps are performed in the order: (a), (b), (c), (d) and (e), optionally repeating steps (b), (c), (d) and (e) one or more times.

In some embodiments, the method includes treating the target polypeptide, protein or peptide with a reagent for modifying a terminal amino acid of the polypeptide, protein or peptide. In some aspects, the reagent for modifying a terminal amino acid of a polypeptide comprises a chemical agent or an enzymatic agent. In some embodiments, the target polypeptide, protein or peptide is contacted with the reagent for modifying a terminal amino acid before step (b). In some embodiments, the target polypeptide, protein or peptide is contacted with the reagent for modifying a terminal amino acid before removing the terminal amino acid.

In some embodiments, the method further includes removing the binding agent after transferring information from the secondary tag to the recording tag. In some aspects, the binding agent is removed after step (d). In some aspects, the binding agent is removed before step (e). In some aspects, removing the binding agent is performed after transferring information from the secondary tag of the adaptor molecule to the recording tag associated with the target.

In some embodiments, the provided methods for analysis of macromolecules can be used in combination with a method for performing a binding reaction that forms a stable complex. In some aspects, the step of contacting the macromolecule with a binding agent capable of binding to the macromolecule further includes components and/or steps for stabilizing the complex. For example, the binding reaction comprises contacting a binding agent with a target macromolecule, wherein the binding agent and the target each comprises or is associated with a stabilizing component, allowing the binding agent to interact with the target, and allowing linking of the stabilizing components to form a stable complex. In some aspects, the stable complex may include the binding agent and associated coding tag, the adaptor molecule, the stabilizing component associated with the binding agent, the target and associated stabilizing component, and optionally a linking agent. In some embodiments, the stabilizing component associated with the binding agent is joined to the coding tag associated with the binding agent.

In some cases, the stabilizing components are linked upon introduction to a linking agent. For example, the linking agent comprises a chemical reagent, a non-biological reagent, a biological reagent, or a combination thereof. In some examples, the linking agent comprises a protein and/or an oligonucleotide. Once activated, the linking of the stabilizing components, either directly with each other or indirectly via a linker (e.g., a linking agent) or other components, allows formation of a stable complex with the binding agent and target. In some embodiments, a stabilizing component is joined or attached (directly or indirectly via a linker) to a nucleic acid molecule or oligonucleotide.

In some embodiments, the linking agent comprises an oligonucleotide comprising a sequence complementary to a nucleic acid joined to the binding agent (e.g., stabilizing component associated with the coding tag) and the adaptor molecule comprises a first hybridization region complementary to the coding tag. In this aspect, a stable complex is formed that includes a recording tag joined to the macromolecule target, the macromolecule target bound by the binding agent associated with a coding tag, the coding tag hybridized to both the adaptor molecule and the linking agent (hybridized to the stabilizing component), and the linking agent is also hybridized to the stabilizing component of the recording tag.

In some embodiments, information is transferred from the coding tag to the linking agent, and this information is subsequently transferred to the recording tag. In some aspects, information is transferred from an adaptor molecule by ligation, wherein the adaptor molecule is part of the linking agent joining the stabilizing component associated with the recording tag with the stabilizing component associated with the binding agent.

In a specific example, a linking agent comprises two DNA pieces joined by a linker (a first DNA for hybridizing to the stabilizing component associated with the recording tag and a second DNA comprising the adaptor molecule with a secondary tag for transferring information to the recording tag). In this example, the secondary tag of the adaptor molecule is ligated to the recording tag, USER enzyme can be used to cleave the U residue and remove the remaining portion of the linking agent from the extended recording tag. In some cases, this approach may be useful for reducing or removing the requirement for spacers in the information transfer step. In some embodiments, single stranded DNA ligation is used for information transfer.

In some embodiments, the macromolecule analysis assay comprises: providing a macromolecule and an associated recording tag joined to a support, wherein the macromolecule is also associated with a stabilizing component; contacting the macromolecule with a binding agent capable of binding to the macromolecule, wherein the binding agent comprises a coding tag with identifying information regarding the binding agent, wherein the binding agent is also associated with a stabilizing component; providing a linking agent and linking the stabilizing components to form a stable complex comprising the macromolecule, the binding agent and the stabilizing components; providing an adaptor molecule comprising a first hybridization sequence substantially complementary to at least a portion of the coding tag, and a secondary tag, to allow hybridization between the adaptor molecule (or the first hybridization sequence) and the coding tag (or the portion of the coding tag); and transferring the information of the secondary tag to the recording tag to generate an extended recording tag; and analyzing the extended recording tag. In some cases, information is transferred from the secondary tag of the adaptor molecule to the recording tag. In some cases, information is transferred from the adaptor molecule to the linking agent, then from the linking agent to the recording tag.

A. Sample and Macromolecule

In some embodiments, the analysis assay is performed on one or more macromolecules of unknown identity that is obtained from a sample. In some cases, the macromolecules are from a mixture of molecules obtained from a sample. A macromolecule can be a large molecule composed of smaller subunits. In certain embodiments, a macromolecule is a protein, a protein complex, polypeptide, peptide, nucleic acid molecule, carbohydrate, lipid, macrocycle, or a chimeric macromolecule. A macromolecule (e.g., protein, polypeptide, peptide) in the methods disclosed herein may be obtained from any suitable source or sample.

The methods disclosed herein can be used for analysis, including detection, identification, quantitation and/or sequencing, of a plurality of macromolecules simultaneously (multiplexing). Multiplexing as used herein refers to analysis of a plurality of macromolecules (e.g. polypeptides) in the same assay. The plurality of macromolecules can be derived from the same sample or different samples. The plurality of macromolecules can be derived from the same subject or different subjects. The plurality of macromolecules that are analyzed can be different macromolecules, or the same macromolecule derived from different samples. A plurality of macromolecules includes 2 or more macromolecules, 5 or more macromolecules, 10 or more macromolecules, 50 or more macromolecules, 100 or more macromolecules, 500 or more macromolecules, 1000 or more macromolecules, 5,000 or more macromolecules, 10,000 or more macromolecules, 50,000 or more macromolecules, 100,000 or more macromolecules, 500,000 or more macromolecules, or 1,000,000 or more macromolecules.

In some embodiments, the target is or comprises macromolecules (e.g., proteins, polypeptides, or peptides) obtained from a sample that is a biological sample. In some embodiments, the sample comprises but is not limited to, mammalian or human cells, yeast cells, and/or bacterial cells. In some embodiments, the sample contains cells that are from a sample obtained from a multicellular organism. For example, the sample may be isolated from an individual. In some embodiments, the sample may comprise a single cell type or multiple cell types. In some embodiments, the sample may be obtained from a mammalian organism or a human, for example by puncture, or other collecting or sampling procedures. In some embodiments, the sample comprises two or more cells.

In some embodiments, the biological sample may contain whole cells and/or live cells and/or cell debris. In some examples, a suitable source or sample, may include but is not limited to: biological samples, such as biopsy samples, cell cultures, cells (both primary cells and cultured cell lines), sample comprising cell organelles or vesicles, tissues and tissue extracts; of virtually any organism. For example, a suitable source or sample, may include but is not limited to: biopsy; fecal matter; bodily fluids (such as blood, whole blood, serum, plasma, urine, lymph, bile, aqueous humor, breast milk, cerumen (earwax), chyle, chyme, endolymph, perilymph, exudates, cerebrospinal fluid, interstitial fluid, aqueous or vitreous humor, colostrum, sputum, amniotic fluid, saliva, anal and vaginal secretions, gastric acid, gastric juice, lymph, mucus (including nasal drainage and phlegm), pericardial fluid, peritoneal fluid, pleural fluid, pus, rheum, saliva, sebum (skin oil), sputum, synovial fluid, perspiration and semen, a transudate, vomit and mixtures of one or more thereof, an exudate (e.g., fluid obtained from an abscess or any other site of infection or inflammation) or fluid obtained from a joint (normal joint or a joint affected by disease such as rheumatoid arthritis, osteoarthritis, gout or septic arthritis) of virtually any organism, with mammalian-derived samples, including microbiome-containing samples, being preferred and human-derived samples, including microbiome-containing samples, being particularly preferred; environmental samples (such as air, agricultural, water and soil samples); microbial samples including samples derived from microbial biofilms and/or communities, as well as microbial spores; tissue samples including tissue sections, research samples including extracellular fluids, extracellular supernatants from cell cultures, inclusion bodies in bacteria, cellular components including mitochondria and cellular periplasm. In some embodiments, the biological sample comprises a body fluid or is derived from a body fluid, wherein the body fluid is obtained from a mammal or a human. In some embodiments, the sample includes bodily fluids, or cell cultures from bodily fluids.

In some embodiments, the macromolecules (e.g., polypeptides and proteins) may be obtained and prepared from a single cell type or multiple cell types. In some embodiments, the sample comprises a population of cells. In some embodiments, the macromolecules (e.g., proteins, polypeptides, or peptides) are from a cellular or subcellular component, an extracellular vesicle, an organelle, or an organized subcomponent thereof. The macromolecules (e.g., proteins, polypeptides, or peptides) may be from organelles, for example, mitochondria, nuclei, or cellular vesicles. In one embodiment, one or more specific types of single cells or subtypes thereof may be isolated. In some embodiments, the sample may include but are not limited to cellular organelles, (e.g., nucleus, golgi apparatus, ribosomes, mitochondria, endoplasmic reticulum, chloroplast, cell membrane, vesicles, etc.).

In certain embodiments, the macromolecule is or comprises a protein, a protein complex, a polypeptide, or peptide. Amino acid sequence information and post-translational modifications of a peptide, polypeptide, or protein are transduced into a nucleic acid encoded library that can be analyzed via next generation sequencing methods. A peptide may comprise L-amino acids, D-amino acids, or both. A peptide, polypeptide, protein, or protein complex may comprise a standard, naturally occurring amino acid, a modified amino acid (e.g., post-translational modification), an amino acid analog, an amino acid mimetic, or any combination thereof. In some embodiments, a peptide, polypeptide, or protein is naturally occurring, synthetically produced, or recombinantly expressed. In any of the aforementioned peptide embodiments, a peptide, polypeptide, protein, or protein complex may further comprise a post-translational modification. Standard, naturally occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). Non-standard amino acids include selenocysteine, pyrrolysine, and N-formylmethionine, β-amino acids, homo-amino acids, Proline and Pyruvic acid derivatives, 3-substituted Alanine derivatives, Glycine derivatives, ring-substituted Phenylalanine and Tyrosine Derivatives, linear core amino acids, and N-methyl amino acids.

A post-translational modification (PTM) of a peptide, polypeptide, or protein may be a covalent modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation (e.g., N-linked, O-linked, C-linked, phosphoglycosylation), glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide, polypeptide, or protein. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini of a peptide, polypeptide, or protein. Post-translational modification can regulate a protein's “biology” within a cell, e.g., its activity, structure, stability, or localization. For example, phosphorylation plays an important role in regulation of protein, particularly in cell signaling (Prabakaran et al., 2012, Wiley Interdiscip Rev Syst Biol Med 4: 565-583). In another example, the addition of sugars to proteins, such as glycosylation, has been shown to promote protein folding, improve stability, and modify regulatory function and the attachment of lipids to proteins enables targeting to the cell membrane. A post-translational modification can also include peptide, polypeptide, or protein modifications to include one or more detectable labels.

In certain embodiments, a peptide, polypeptide, or protein can be fragmented. Peptides, polypeptides, or proteins can be fragmented by any means known in the art, including fragmentation by a protease or endopeptidase. In some embodiments, fragmentation of a peptide, polypeptide, or protein is targeted by use of a specific protease or endopeptidase. A specific protease or endopeptidase binds and cleaves at a specific consensus sequence (e.g., TEV protease). In other embodiments, fragmentation of a peptide, polypeptide, or protein is non-targeted or random by use of a non-specific protease or endopeptidase. A non-specific protease may bind and cleave at a specific amino acid residue rather than a consensus sequence (e.g., proteinase K is a non-specific serine protease). In some embodiments, proteinases and endopeptidases, such as those known in the art, can be used to cleave a protein or polypeptide into smaller peptide fragments include proteinase K, trypsin, chymotrypsin, pepsin, thermolysin, thrombin, Factor Xa, furin, endopeptidase, papain, pepsin, subtilisin, elastase, enterokinase, Genenase™ I, Endoproteinase LysC, Endoproteinase AspN, Endoproteinase GluC, etc. (Granvogl et al., 2007, Anal Bioanal Chem 389: 991-1002). In certain embodiments, a peptide, polypeptide, or protein is fragmented by proteinase K, or optionally, a thermolabile version of proteinase K to enable rapid inactivation. In some cases, Proteinase K is stable in denaturing reagents, such as urea and SDS, and enables digestion of completely denatured proteins. Protein and polypeptide fragmentation into peptides can be performed before or after attachment of a DNA tag or DNA recording tag.

Chemical reagents can also be used to digest proteins into peptide fragments. A chemical reagent may cleave at a specific amino acid residue (e.g., cyanogen bromide hydrolyzes peptide bonds at the C-terminus of methionine residues). Chemical reagents for fragmenting polypeptides or proteins into smaller peptides include cyanogen bromide (CNBr), hydroxylamine, hydrazine, formic acid, BNPS-skatole [2-(2-nitrophenylsulfenyl) methylindole], iodosobenzoic acid, ⋅NTCB+Ni (2-nitro-5-thiocyanobenzoic acid), etc.

In certain embodiments, following enzymatic or chemical cleavage, the resulting peptide fragments are approximately the same desired length, e.g., from about 10 amino acids to about 70 amino acids, from about 10 amino acids to about 60 amino acids, from about 10 amino acids to about 50 amino acids, about 10 to about 40 amino acids, from about 10 to about 30 amino acids, from about 20 amino acids to about 70 amino acids, from about 20 amino acids to about 60 amino acids, from about 20 amino acids to about 50 amino acids, about 20 to about 40 amino acids, from about 20 to about 30 amino acids, from about 30 amino acids to about 70 amino acids, from about 30 amino acids to about 60 amino acids, from about 30 amino acids to about 50 amino acids, or from about 30 amino acids to about 40 amino acids. A cleavage reaction may be monitored, preferably in real time, by spiking the protein or polypeptide sample with a short test FRET (fluorescence resonance energy transfer) peptide comprising a peptide sequence containing a proteinase or endopeptidase cleavage site. In the intact FRET peptide, a fluorescent group and a quencher group are attached to either end of the peptide sequence containing the cleavage site, and fluorescence resonance energy transfer between the quencher and the fluorophore leads to low fluorescence. Upon cleavage of the test peptide by a protease or endopeptidase, the quencher and fluorophore are separated giving a large increase in fluorescence. A cleavage reaction can be stopped when a certain fluorescence intensity is achieved, allowing a reproducible cleavage endpoint to be achieved.

In some aspects, a sample of macromolecules (e.g., peptides, polypeptides, or proteins) can undergo protein fractionation methods where proteins or peptides are separated by one or more properties such as cellular location, molecular weight, hydrophobicity, isoelectric point, or protein enrichment methods. In some embodiments, a subset of macromolecules (e.g., proteins) within a sample is fractionated such that a subset of the macromolecules is sorted from the rest of the sample. For example, the sample may undergo fractionation methods prior to attachment to a support. Alternatively, or additionally, protein enrichment methods may be used to select for a specific protein or peptide (see, e.g., Whiteaker et al., 2007, Anal. Biochem. 362:44-54, incorporated by reference in its entirety) or to select for a particular post translational modification (see, e.g., Huang et al., 2014. J. Chromatogr. A 1372:1-17, incorporated by reference in its entirety). Alternatively, a particular class or classes of proteins such as immunoglobulins, or immunoglobulin (Ig) isotypes such as IgG, can be affinity enriched or selected for analysis. In the case of immunoglobulin molecules, analysis of the sequence and abundance or frequency of hypervariable sequences involved in affinity binding are of particular interest, particularly as they vary in response to disease progression or correlate with healthy, immune, and/or or disease phenotypes. Overly abundant proteins can also be subtracted from the sample using standard immunoaffinity methods. Depletion of abundant proteins can be useful for plasma samples where over 80% of the protein constituent is albumin and immunoglobulins. Several commercial products are available for depletion of plasma samples of overly abundant proteins, including depletion spin columns that remove top 2-20 plasma proteins (Pierce, Agilent), or PROTIA and PROT20 (Sigma-Aldrich).

In certain embodiments, a protein sample dynamic range can be modulated by fractionating the protein sample using standard fractionation methods, including electrophoresis and liquid chromatography (Zhou et al., 2012, Anal Chem 84(2): 720-734), or partitioning the fractions into compartments (e.g., droplets) loaded with limited capacity protein binding beads/resin (e.g. hydroxylated silica particles) (McCormick, 1989, Anal Biochem 181(1): 66-74) and eluting bound protein. Excess protein in each compartmentalized fraction is washed away. Examples of electrophoretic methods include capillary electrophoresis (CE), capillary isoelectric focusing (CIEF), capillary isotachophoresis (CITP), free flow electrophoresis, gel-eluted liquid fraction entrapment electrophoresis (GELFrEE). Examples of liquid chromatography protein separation methods include reverse phase (RP), ion exchange (IE), size exclusion (SE), hydrophilic interaction, etc. Examples of compartment partitions include emulsions, droplets, microwells, physically separated regions on a flat substrate, etc. Exemplary protein binding beads/resins include silica nanoparticles derivatized with phenol groups or hydroxyl groups (e.g., StrataClean Resin from Agilent Technologies, RapidClean from LabTech, etc.). By limiting the binding capacity of the beads/resin, highly-abundant proteins eluting in a given fraction will only be partially bound to the beads, and excess proteins removed.

In some embodiments, a partition barcode is used which comprises assignment of a unique barcode to a subsampling of macromolecules from a population of macromolecules within a sample. This partition barcode may be comprised of identical barcodes arising from the partitioning of macromolecules within compartments labeled with the same barcode (e.g., a barcoded bead population in which multiple beads share the same barcode). The use of physical compartments effectively subsamples the original sample to provide assignment of partition barcodes. For instance, a set of beads labeled with 10,000 different compartment barcodes is provided. Furthermore, suppose in a given assay, that a population of 1 million beads are used in the assay. On average, there are 100 beads per compartment barcode (Poisson distribution). Further suppose that the beads capture an aggregate of 10 million macromolecules. On average, there are 10 macromolecules per bead, with 100 compartments per compartment barcode, there are effectively 1,000 macromolecules per partition barcode (comprised of 100 compartment barcodes for 100 distinct physical compartments).

In another embodiment, single molecule partitioning and partition barcoding of polypeptides is accomplished by labeling polypeptides (chemically or enzymatically) with an amplifiable DNA UMI tag (e.g., recording tag) at the N or C terminus, or both. DNA tags are attached to the body of the polypeptide (internal amino acids) via non-specific photo-labeling or specific chemical attachment to reactive amino acids such as lysines. Information from the recording tag attached to the terminus of the peptide is transferred to the DNA tags via an enzymatic emulsion PCR (Williams et al., Nat Methods, (2006) 3(7):545-550; Schutze et al., Anal Biochem. (2011) 410(1):155-157) or emulsion in vitro transcription/reverse transcription (IVT/RT) step. In the preferred embodiment, a nanoemulsion is employed such that, on average, there is fewer than a single polypeptide per emulsion droplet with size from 50 nm-1000 nm (Nishikawa et al., J Nucleic Acids. (2012) 2012: 923214; Gupta et al., Soft Matter. (2016) 12(11):2826-41; Sole et al., Langmuir (2006, 22(20):8326-8332). Additionally, all the components of PCR are included in the aqueous emulsion mix including primers, dNTPs, Mg2+, polymerase, and PCR buffer. If IVT/RT is used, then the recording tag is designed with a T7/SP6 RNA polymerase promoter sequence to generate transcripts that hybridize to the DNA tags attached to the body of the polypeptide (Ryckelynck et al., RNA. (2015) 21(3):458-469). A reverse transcriptase (RT) copies the information from the hybridized RNA molecule to the DNA tag. In this way, emulsion PCR or IVT/RT can be used to effectively transfer information from the terminus recording tag to multiple DNA tags attached to the body of the polypeptide.

In some embodiments, a sample of macromolecule targets (e.g., peptides, polypeptides, or proteins) can be processed into a physical area or volume e.g., into a compartment. Various processing and/or labeling steps may be performed on the sample. In some embodiments, the compartment separates or isolates a subset of macromolecules from a sample of macromolecules. In some examples, the compartment may be an aqueous compartment (e.g., microfluidic droplet), a solid compartment (e.g., picotiter well or microtiter well on a plate, tube, vial, bead), or a separated region on a surface. In some cases, a compartment may comprise one or more beads to which macromolecules may be immobilized. In some embodiments, macromolecules in a compartment is labeled with a compartment tag including a barcode. For example, the macromolecules in one compartment can be labeled with the same barcode or macromolecules in multiple compartments can be labeled with the same barcode. See e.g., Valihrach et al., Int J Mol Sci. 2018 Mar. 11; 19(3). pii: E807. Encapsulation of cellular contents via gelation in beads is a useful approach to single cell analysis (Tamminen et al., Front Microbiol (2015) 6: 195; Spencer et al., ISME J (2016) 10(2): 427-436). Barcoding single cell droplets enables all components from a single cell to be labeled with the same identifier (Klein et al., Cell (2015) 161(5): 1187-1201; Zilionis et al., Nat Protoc (2017) 12(1): 44-73; International Patent Publication No. WO 2016/130704). Compartment barcoding can be accomplished in a number of ways including direct incorporation of unique barcodes into each droplet by droplet joining (Bio-Rad Laboratories), by introduction of barcoded beads into droplets (10× Genomics), or by combinatorial barcoding of components of the droplet post encapsulation and gelation using and split-pool combinatorial barcoding as described by Gunderson et al. (International Patent Publication No. WO 2016/130704, incorporated by reference in its entirety). A similar combinatorial labeling scheme can also be applied to nuclei (Vitak et al., Nat Methods (2017) 14(3):302-308).

In some embodiments, the macromolecule is joined to a support before contacting with a binding agent. In some cases, it is desirable to use a support with a large carrying capacity to immobilize a large number of macromolecules in a sample. In some embodiments, it is preferred to immobilize the macromolecules using a three-dimensional support (e.g., a porous matrix or a bead). In some examples, the preparation includes joining the macromolecule to nucleic acid molecule or a oligonucleotide prior to or after immobilizing the macromolecule. In some embodiments, a plurality of macromolecules is attached to a support prior to contacting with a binding agent.

A support can be any solid or porous support including, but not limited to, a bead, a microbead, an array, a glass surface, a silicon surface, a plastic surface, a filter, a membrane, a PTFE membrane, nylon, a microtiter well, an ELISA plate, a spinning interferometry disc, a nitrocellulose membrane, a nitrocellulose-based polymer surface, a nanoparticle, or a microsphere. Materials for a support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, silica, polyanhydrides, polyglycolic acid, polyvinylchloride, polylactic acid, polyorthoesters, functionalized silane, polypropylfumerate, collagen, glycosaminoglycans, polyamino acids, or any combination thereof. In certain embodiments, a support is a bead, for example, a polystyrene bead, a polymer bead, a polyacrylate bead, an agarose bead, a cellulose bead, a dextran bead, an acrylamide bead, a solid core bead, a porous bead, a paramagnetic bead, a glass bead, a silica-based bead, or a controlled pore bead, or any combinations thereof. In some specific embodiments, the support is a porous agarose bead.

In some embodiments, the support may comprise any suitable solid material, including porous and non-porous materials, to which a macromolecule, e.g., a polypeptide, can be associated directly or indirectly, by any means known in the art, including covalent and non-covalent interactions, or any combination thereof. A support may be two-dimensional (e.g., planar surface) or three-dimensional (e.g., gel matrix or bead). A support can be any support surface including, but not limited to, a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a microtiter well, an ELISA plate, a spinning interferometry disc, a polymer matrix, a nanoparticle, or a microsphere. Materials for a support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, or a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

Various reactions may be used to attach the macromolecules to a support (e.g., a solid or a porous support The macromolecules may be attached directly or indirectly to the support. In some cases, the macromolecules are attached to the support via a nucleic acid. Exemplary reactions include the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO)); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing macromolecules (e.g., polypeptides) to a support since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction. In one case, a polypeptide is labeled with a bifunctional click chemistry reagent, such as alkyne-NHS ester (acetylene-PEG-NETS ester) reagent or alkyne-benzophenone to generate an alkyne-labeled polypeptide. In some embodiments, an alkyne can also be a strained alkyne, such as cyclooctynes including Dibenzocyclooctyl (DBCO), etc.

In certain embodiments where multiple macromolecules are immobilized on the same support, the macromolecules can be spaced appropriately to accommodate the analysis steps to be used to assess the target. For example, it may be advantageous to space the macromolecules that optimally to allow a nucleic acid-based method for assessing and sequencing the proteins to be performed. In some cases, spacing of the macromolecules on the support is determined based on the consideration that information transfer from an adaptor molecule hybridized to the coding tag of a binding agent bound to one immobilized macromolecule may reach a neighboring macromolecule.

In some embodiments, the surface of the support is passivated (blocked). A “passivated” surface refers to a surface that has been treated with outer layer of material. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), diamond-like carbon (DLC), DLC+PEG (Stavis et al., 2011, Proc. Natl. Acad. Sci. USA 108:983-988), and zwitterionic moiety (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol (PVA), and proteins like BSA and casein. Alternatively, density of macromolecules (e.g., proteins, polypeptide, or peptides) can be titrated on the surface or within the volume of a solid substrate by spiking a competitor or “dummy” reactive molecule when immobilizing the proteins, polypeptides or peptides to the solid substrate.

To control spacing of the immobilized targets on the support, the density of functional coupling groups for attaching the target (e.g., TCO or carboxyl groups (COOH)) may be titrated on the substrate surface. In some embodiments, multiple target molecules (e.g., macromolecules) are spaced apart on the surface or within the volume (e.g., porous supports) of a support such that adjacent molecules are spaced apart at a distance of about 50 nm to about 500 nm, or about 50 nm to about 400 nm, or about 50 nm to about 300 nm, or about 50 nm to about 200 nm, or about 50 nm to about 100 nm. In some embodiments, multiple molecules are spaced apart on the surface of a support with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm. In some embodiments, multiple molecules are spaced apart on the surface of a support with an average distance of at least 50 nm. In some embodiments, molecules are spaced apart on the surface or within the volume of a support such that, empirically, the relative frequency of inter- to intra-molecular events (e.g. transfer of information) is <1:10; <1:100; <1:1,000; or <1:10,000.

In some embodiments, the plurality of macromolecules is coupled on the support spaced apart at an average distance between two adjacent molecules which ranges from about 50 to 100 nm, from about 50 to 250 nm, from about 50 to 500 nm, from about 50 to 750 nm, from about 50 to 1,000 nm, from about 50 to 1,500 nm, from about 50 to 2,000 nm, from about 100 to 250 nm, from about 100 to 500 nm, from about 200 to 500 nm, from about 300 to 500 nm, from about 100 to 1000 nm, from about 500 to 600 nm, from about 500 to 700 nm, from about 500 to 800 nm, from about 500 to 900 nm, from about 500 to 1,000 nm, from about 500 to 2,000 nm, from about 500 to 5,000 nm, from about 1,000 to 5,000 nm, or from about 3,000 to 5,000 nm.

In some embodiments, appropriate spacing of the macromolecules on the support is accomplished by titrating the ratio of available attachment molecules on the substrate surface. In some examples, the substrate surface (e.g., bead surface) is functionalized with a carboxyl group (COOH) which is treated with an activating agent (e.g., activating agent is EDC and Sulfo-NHS). In some examples, the substrate surface (e.g., bead surface) comprises NHS moieties. In some embodiments, a mixture of mPEGₙ-NH₂ and NH₂-PEGₙ-mTet is added to the activated beads (wherein n is any number, such as 1-100). The ratio between the mPEG₃-NH₂ (not available for coupling) and NH₂-PEG₂₄-mTet (available for coupling) is titrated to generate an appropriate density of functional moieties available to attach the polypeptides on the substrate surface. In certain embodiments, the mean spacing between coupling moieties (e.g., NH₂-PEG₄-mTet) on the solid surface is at least 50 nm, at least 100 nm, at least 250 nm, or at least 500 nm. In some specific embodiments, the ratio of NH₂-PEGₙ-mTet to mPEG₃-NH₂ is about or greater than 1:1000, about or greater than 1:10,000, about or greater than 1:100,000, or about or greater than 1:1,000,000. In some further embodiments, the recording tag attaches to the NH₂-PEGₙ-mTet. In some embodiments, the spacing of the macromolecules on the support is achieved by controlling the concentration and/or number of available COOH or other functional groups on the support.

B. Amino Acid Cleavage

In embodiments relating to methods of analyzing peptides or polypeptides using a degradation based approach, following contacting and binding of a first binding agent to an n NTAA of a peptide of n amino acids, hybridizing with adaptor molecules, and transferring of the secondary tag information to a nucleic acid associated with the peptide thereby generating a first order extended nucleic acid (e.g., on the recording tag), the n NTAA is eliminated. Removal of the n labeled NTAA by contacting with an enzyme or chemical reagents converts the n−1 amino acid of the peptide to an N-terminal amino acid, which is referred to herein as an n−1 NTAA. A second binding agent is contacted with the peptide or polypeptides and binds to the n−1 NTAA, adaptor molecules are hybridized, and the second binding agent's information is transferred from the secondary tag to the first order extended nucleic acid thereby generating a second order extended nucleic acid (e.g., for generating a concatenated n^{th} order extended nucleic acid representing the peptide). Elimination of the n−1 labeled NTAA converts the n−2 amino acid of the peptide or polypeptides to an N-terminal amino acid, which is referred to herein as n−2 NTAA. Additional binding, hybridizing, transferring information, and removal, can occur as described above up to n amino acids to generate an n^{th} order extended nucleic acid or n separate extended nucleic acids, which collectively represent the peptide or polypeptides. As used herein, an n “order” when used in reference to a binding agent, coding tag, or extended nucleic acid, refers to the n binding cycle, wherein the binding agent and its associated coding tag is used or the n binding cycle where the extended nucleic acid is created (e.g. on recording tag). In some embodiments, steps including the NTAA in the described exemplary approach can be performed instead with a C terminal amino acid (CTAA).

In certain embodiments relating to analyzing peptides or polypeptides, the terminal amino acid is removed or cleaved from the peptide or polypeptides to expose a new terminal amino acid. In some embodiments, the terminal amino acid is an NTAA. In other embodiments, the terminal amino acid is a CTAA. Cleavage of a terminal amino acid can be accomplished by any number of known techniques, including chemical cleavage and enzymatic cleavage. In some embodiments, an engineered enzyme that catalyzes or reagent that promotes the removal of the PITC-derivatized or other labeled N-terminal amino acid is used. In some embodiments, the terminal amino acid is removed or eliminated using any of the methods as described in International Patent Publications No. WO 2020/223133, WO 2020/198264 or in US 20200348307 A1.

In some embodiments, the reagent for removing a terminal amino acid includes a carboxypeptidase or an aminopeptidase or a variant, mutant, or modified protein thereof a hydrolase or a variant, mutant, or modified protein thereof; a mild Edman degradation reagent; an Edmanase enzyme; anhydrous TFA, a base; or any combination thereof. In some embodiments, the mild Edman degradation uses a dichloro or monochloro acid; the mild Edman degradation uses TFA, TCA, or DCA; or the mild Edman degradation uses triethylamine, triethanolamine, or triethylammonium acetate (Et₃NHOAc).

In some cases, the reagent for removing the amino acid comprises a base. In some embodiments, the base is a hydroxide, an alkylated amine, a cyclic amine, a carbonate buffer, trisodium phosphate buffer, or a metal salt. In some examples, the hydroxide is sodium hydroxide; the alkylated amine is selected from methylamine, ethylamine, propylamine, dimethylamine, diethylamine, dipropylamine, trimethylamine, triethylamine, tripropylamine, cyclohexylamine, benzylamine, aniline, diphenylamine, N,N-Diisopropylethylamine (DIPEA), and lithium diisopropylamide (LDA); the cyclic amine is selected from pyridine, pyrimidine, imidazole, pyrrole, indole, piperidine, prolidine, 1,8-diazabicyclo[5.4.0]undec-7-ene (DBU), and 1,5-diazabicyclo[4.3.0]non-5-ene (DBN); the carbonate buffer comprises sodium carbonate, potassium carbonate, calcium carbonate, sodium bicarbonate, potassium bicarbonate, or calcium bicarbonate; the metal salt comprises silver; or the metal salt is AgClO₄.

Enzymatic cleavage of a NTAA may be accomplished by an aminopeptidase or other peptidases. Aminopeptidases naturally occur as monomeric and multimeric enzymes, and may be metal or ATP-dependent. Natural aminopeptidases have very limited specificity, and generically cleave N-terminal amino acids in a processive manner, cleaving one amino acid off after another. For the methods described here, aminopeptidases (e.g., metalloenzymatic aminopeptidase) may be engineered to possess specific binding or catalytic activity to the NTAA only when modified with an N-terminal label. For example, an aminopeptidase may be engineered such than it only cleaves an N-terminal amino acid if it is modified by a group such as PTC, modified-PTC, Cbz, DNP, SNP, acetyl, guanidinyl, diheterocyclic methanimine, etc. In this way, the aminopeptidase cleaves only a single amino acid at a time from the N-terminus, and allows control of the degradation cycle. In some embodiments, the modified aminopeptidase is non-selective as to amino acid residue identity while being selective for the N-terminal label. In other embodiments, the modified aminopeptidase is selective for both amino acid residue identity and the N-terminal label. Engineered aminopeptidase mutants that bind to and cleave individual or small groups of labelled (biotinylated) NTAAs have been described (see, PCT Publication No. WO2010/065322).

Engineered aminopeptidase mutants that bind to and cleave individual or small groups of labelled (biotinylated) NTAAs have been described (see, PCT Publication No. WO2010/065322, incorporated by reference in its entirety). Aminopeptidases are enzymes that cleave amino acids from the N-terminus of proteins or peptides. Natural aminopeptidases have very limited specificity, and generically eliminate N-terminal amino acids in a processive manner, cleaving one amino acid off after another (Kishor et al., 2015, Anal. Biochem. 488:6-8). However, residue specific aminopeptidases have been identified (Eriquez et al., J. Clin. Microbiol. 1980, 12:667-71; Wilce et al., 1998, Proc. Natl. Acad. Sci. USA 95:3472-3477; Liao et al., 2004, Prot. Sci. 13:1802-10). Aminopeptidases may be engineered to specifically bind to 20 different NTAAs representing the standard amino acids that are labeled with a specific moiety (e.g., PTC, DNP, SNP, etc.). Control of the stepwise degradation of the N-terminus of the peptide is achieved by using engineered aminopeptidases that are only active (e.g., binding activity or catalytic activity) in the presence of the label. In another example, Havranak et al. (U.S. Patent Publication No. US 2014/0273004) describes engineering aminoacyl tRNA synthetases (aaRSs) as specific NTAA binders. The amino acid binding pocket of the aaRSs has an intrinsic ability to bind cognate amino acids, but generally exhibits poor binding affinity and specificity. Moreover, these natural amino acid binders don't recognize N-terminal labels. Directed evolution of aaRS scaffolds can be used to generate higher affinity, higher specificity binding agents that recognized the N-terminal amino acids in the context of an N-terminal label.

In certain embodiments, the aminopeptidase may be engineered to be non-specific, such that it does not selectively recognize one particular amino acid over another, but rather just recognizes the labeled N-terminus. In yet another embodiment, cyclic cleavage is attained by using an engineered acylpeptide hydrolase (APH) to cleave an acetylated NTAA. In yet another embodiment, amidination (guanidinylation) of the NTAA is employed to enable mild cleavage of the labeled NTAA using NaOH (Hamada, (2016) Bioorg Med Chem Lett 26(7): 1690-1695).

In some embodiments, the method further comprises contacting the polypeptide with a proline aminopeptidase under conditions suitable to cleave an N-terminal proline before step (b). In some examples, a proline aminopeptidase (PAP) is an enzyme that is capable of specifically cleaving an N-terminal proline from a polypeptide. PAP enzymes that cleave N-terminal prolines are also referred to as proline iminopeptidases (PIPs). Known monomeric PAPs include family members from B. coagulans, L. delbrueckii, L. gonorrhoeae, F. meningosepticum, S. marcescens, T. acidophilum, L. plantarum (MEROPS S33.001) Nakajima et al., J Bacteriol. (2006) 188(4):1599-606; Kitazono et al., Bacteriol (1992) 174(24):7919-7925). Known multimeric PAPs including D. hansenii (Bolumar et al., (2003) 86(1-2):141-151) and similar homologues from other species (Basten et al., Mol Genet Genomics (2005) 272(6):673-679). Either native or engineered variants/mutants of PAPs may be employed.

For embodiments relating to CTAA binding agents, methods of cleaving CTAA from peptides or polypeptides are also known in the art. For example, U.S. Pat. No. 6,046,053 discloses a method of reacting the peptide or protein with an alkyl acid anhydride to convert the carboxy-terminal into oxazolone, liberating the C-terminal amino acid by reaction with acid and alcohol or with ester. Enzymatic cleavage of a CTAA may also be accomplished by a carboxypeptidase. Several carboxypeptidases exhibit amino acid preferences, e.g., carboxypeptidase B preferentially cleaves at basic amino acids, such as arginine and lysine. As described above, carboxypeptidases may also be modified in the same fashion as aminopeptidases to engineer carboxypeptidases that specifically bind to CTAAs having a C-terminal label. In this way, the carboxypeptidase cleaves only a single amino acid at a time from the C-terminus, and allows control of the degradation cycle. In some embodiments, the modified carboxypeptidase is non-selective as to amino acid residue identity while being selective for the C-terminal label. In other embodiments, the modified carboxypeptidase is selective for both amino acid residue identity and the C-terminal label.

C. Analysis

In some embodiments, the extended recording tag generated from performing the provided methods comprises information transferred from one or more secondary tags. In some embodiments, the extended recording tags further comprise identifying information regarding one or more binding agents and binding events. In some embodiments, the extended recording tags are amplified (or a portion thereof) prior to determining at least the sequence of the extended recording tag. In some embodiments, the extended recording tags (or a portion thereof) are released prior to determining at least the sequence of extended recording tag. In some embodiments, the transferred secondary sequences are analyzed.

The extended nucleic acid (e.g., recording tag) is any nucleic acid molecule or sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) that comprises identifying information for a macromolecule, e.g., a polypeptide. In certain embodiments, after a binding agent binds to a polypeptide, information regarding a binding agent can be transferred to the nucleic acid associated with the polypeptide while the binding agent is bound to the polypeptide. In certain embodiments, the information is transferred while the adaptor molecule is hybridized to both the coding tag and the recording tag (or portions thereof).

An extended nucleic acid associated with the macromolecule, e.g., the peptide or polypeptides, with identifying information from the secondary tag may comprise information regarding one or more binding agent(s) via the secondary tag, representing each binding cycle performed. However, in some cases, an extended nucleic acid may also experience a “missed” binding cycle, e.g., if a binding agent fails to bind to the polypeptide, because the adaptor molecule was not hybridized, because the primer extension reaction failed. Thus, an extended nucleic acid may represent 100%, or up to 95%, 90%, 85%, 80%, 75%, 70%, 65%, 60%, 65%, 55%, 50%, 45%, 40%, 35%, 30%, or any subrange thereof, of binding events that have occurred on its associated polypeptide. Moreover, the secondary tag information present in the extended nucleic acid may have at least 30%, 35%, 40%, 45%, 50%, 55%, 60%, 65%, 70%, 75%, 80%, 85%, 90%, 95%, or 100% identity to the corresponding secondary tags.

In certain embodiments, an extended recording tag associated with the immobilized peptide may comprise information from multiple secondary tags representing multiple, successive binding events. In these embodiments, a single, concatenated extended recording tag associated with the immobilized peptide or polypeptides can be representative of a single polypeptide. As referred to herein, transfer of secondary tag information to the recording tag associated with the immobilized peptide or polypeptides also includes transfer to an extended recording tag as would occur in methods involving multiple, successive binding events. Universal priming sequences may also be included in extended nucleic acids on the recording tag associated with the immobilized peptide or polypeptides for amplification and NGS sequencing.

In some examples, the final extended recording tag containing information from one or more binding agents is optionally flanked by universal priming sites to facilitate downstream amplification and/or DNA sequencing. The forward universal priming site (e.g., Illumina's P5-S1 sequence) can be part of the original design of the recording tag and the reverse universal priming site (e.g., Illumina's P7-S2′ sequence) can be added as a final step in the extension of the nucleic acid.

The length of the final extended recording tag generated by the methods described herein is dependent upon multiple factors, including the length of the secondary tag(s) (e.g., barcode and spacer), the length of the nucleic acids (e.g., optionally including any unique molecular identifier, spacer, universal priming site, barcode, or combinations thereof). After transfer of the final tag information to the extended nucleic acid, the tag can be capped by addition of a universal reverse priming site via ligation, primer extension or other methods known in the art. In some embodiments, the universal forward priming site in the nucleic acid (e.g., on the recording tag) is compatible with the universal reverse priming site that is appended to the final extended nucleic acid. In some embodiments, a universal reverse priming site is an Illumina P7 primer (5′-CAAGCAGAAGACGGCATACGAGAT-3′-SEQ ID NO:2) or an Illumina P5 primer (5′-AATGATACGGCGACCACCGA-3′-SEQ ID NO:1). The sense or antisense P7 may be appended, depending on strand sense of the nucleic acid to which the identifying information from the secondary tag is transferred to. An extended nucleic acid library can be cleaved or amplified directly from the support (e.g., beads) and used in traditional next generation sequencing assays and protocols.

In some embodiments, a primer extension reaction is performed on a library of single stranded extended nucleic acids (e.g., extended on the recording tag) to copy complementary strands thereof. In some embodiments, the peptide or polypeptides sequencing assay (e.g., ProteoCode assay), comprises several chemical and enzymatic steps in a cyclical progression.

Extended nucleic acid recording tags can be processed and analysed using a variety of nucleic acid sequencing methods. In some embodiments, extended recording tags containing the information from one or more secondary tags and any other nucleic acid components are processed and analysed. In some embodiments, the collection of extended recording tags can be concatenated. In some embodiments, the extended recording tag can be amplified prior to determining the sequence.

In some embodiments, the recording tag or extended recording tag comprises information from one or more secondary tags is analysed and/or sequenced. In some embodiments, the method includes analyzing the identifying information regarding the binding agent of the macromolecule analysis assay transferred to the recording tag.

Examples of sequencing methods include, but are not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing, duplex interrupted sequencing, and direct imaging of DNA using advanced microscopy.

Suitable sequencing methods for use in the invention include, but are not limited to, the following methods known in the art, such as sequencing by hybridization, sequencing by synthesis technology (e.g., HiSeg™ and Solexa™, Illumina), SMRT™ (Single Molecule Real Time) technology (Pacific Biosciences), true single molecule sequencing (e.g., HeliScope™ Helicos Biosciences), massively parallel next generation sequencing (e.g., SOLiD™, Applied Biosciences; Solexa and HiSeg™, Illumina), massively parallel semiconductor sequencing (e.g., Ion Torrent), pyrosequencing technology (e.g., GS FLX and GS Junior Systems, Roche/454), nanopore sequence (e.g., Oxford Nanopore Technologies).

A library of nucleic acids (e.g., extended nucleic acids) may be amplified in a variety of ways. A library of nucleic acids (e.g., recording tags comprising information from one or more secondary tags) undergo exponential amplification, e.g., via PCR or emulsion PCR. Emulsion PCR is known to produce more uniform amplification (Hori, Fukano et al., Biochem Biophys Res Commun (2007) 352(2): 323-328). Alternatively, a library of nucleic acids (e.g., extended nucleic acids) may undergo linear amplification, e.g., via in vitro transcription of template DNA using T7 RNA polymerase. The library of nucleic acids (e.g., extended nucleic acids) can be amplified using primers compatible with the universal forward priming site and universal reverse priming site contained therein. A library of nucleic acids (e.g., the recording tag) can also be amplified using tailed primers to add sequence to either the 5′-end, 3′-end or both ends of the extended nucleic acids. Sequences that can be added to the termini of the extended nucleic acids include library specific index sequences to allow multiplexing of multiple libraries in a single sequencing run, adaptor sequences, read primer sequences, or any other sequences for making the library of extended nucleic acids compatible for a sequencing platform. An example of a library amplification in preparation for next generation sequencing is as follows: a 20 μl PCR reaction volume is set up using an extended nucleic acid library eluted from ˜1 mg of beads (˜10 ng), 200 μM dNTP, 1 μM of each forward and reverse amplification primers, 0.5 μl (1 U) of Phusion Hot Start enzyme (New England Biolabs) and subjected to the following cycling conditions: 98° C. for 30 sec followed by 20 cycles of 98° C. for 10 sec, 60° C. for 30 sec, 72° C. for 30 sec, followed by 72° C. for 7 min, then hold at 4° C.

In certain embodiments, either before, during or following amplification, the library of nucleic acids (e.g., extended nucleic acids) can undergo target enrichment. In some embodiments, target enrichment can be used to selectively capture or amplify extended nucleic acids representing macromolecules (e.g., polypeptides) of interest from a library of extended nucleic acids before sequencing. In some aspects, target enrichment for protein sequencing is challenging because of the high cost and difficulty in producing highly-specific binding agents for target proteins. In some cases, antibodies are notoriously non-specific and difficult to scale production across thousands of proteins. In some embodiments, the methods of the present disclosure circumvent this problem by converting the protein code into a nucleic acid code which can then make use of a wide range of targeted DNA enrichment strategies available for DNA libraries. In some cases, peptides of interest can be enriched in a sample by enriching their corresponding extended nucleic acids. Methods of targeted enrichment are known in the art, and include hybrid capture assays, PCR-based assays such as TruSeq custom Amplicon (Illumina), padlock probes (also referred to as molecular inversion probes), and the like (see, Mamanova et al., (2010) Nature Methods 7: 111-118; Bodi et al., J. Biomol. Tech. (2013) 24:73-86; Ballester et al., (2016) Expert Review of Molecular Diagnostics 357-372; Mertes et al., (2011) Brief Funct. Genomics 10:374-386; Nilsson et al., (1994) Science 265:2085-8; each of which are incorporated herein by reference in their entirety).

In one embodiment, a library of nucleic acids (e.g., extended recording tags) is enriched via a hybrid capture-based assay. In a hybrid-capture based assay, the library of extended nucleic acids is hybridized to target-specific oligonucleotides that are labelled with an affinity tag (e.g., biotin). Extended nucleic acids hybridized to the target-specific oligonucleotides are “pulled down” via their affinity tags using an affinity ligand (e.g., streptavidin coated beads), and background (non-specific) extended nucleic acids are washed away. The enriched extended nucleic acids (e.g., extended nucleic acids) are then obtained for positive enrichment (e.g., eluted from the beads). In some embodiments, oligonucleotides complementary to the corresponding extended nucleic acid library representations of peptides of interest can be used in a hybrid capture assay. In some embodiments, sequential rounds or enrichment can also be carried out, with the same or different bait sets.

To enrich the entire length of a polypeptide in a library of extended nucleic acids representing fragments thereof (e.g., peptides), “tiled” bait oligonucleotides can be designed across the entire nucleic acid representation of the protein.

In another embodiment, primer extension and ligation-based mediated amplification enrichment (AmpliSeq, PCR, TruSeq TSCA, etc.) can be used to select and module fraction enriched of library elements representing a subset of polypeptides. Competing oligonucleotides can also be employed to tune the degree of primer extension, ligation, or amplification. In the simplest implementation, this can be accomplished by having a mix of target specific primers comprising a universal primer tail and competing primers lacking a 5′ universal primer tail. After an initial primer extension, only primers with the 5′ universal primer sequence can be amplified. The ratio of primer with and without the universal primer sequence controls the fraction of target amplified. In other embodiments, the inclusion of hybridizing but non-extending primers can be used to modulate the fraction of library elements undergoing primer extension, ligation, or amplification.

Targeted enrichment methods can also be used in a negative selection mode to selectively remove extended nucleic acids from a library before sequencing. Examples of undesirable extended nucleic acids that can be removed are those representing over abundant polypeptide species, e.g., for proteins, albumin, immunoglobulins, etc.

A competitor oligonucleotide bait, hybridizing to the target but lacking a biotin moiety, can also be used in the hybrid capture step to modulate the fraction of any particular locus enriched. The competitor oligonucleotide bait competes for hybridization to the target with the standard biotinylated bait effectively modulating the fraction of target pulled down during enrichment. The ten orders dynamic range of protein expression can be compressed by several orders using this competitive suppression approach, especially for the overly abundant species such as albumin. Thus, the fraction of library elements captured for a given locus relative to standard hybrid capture can be modulated from 100% down to 0% enrichment.

Additionally, library normalization techniques can be used to remove overly abundant species from the extended nucleic acid library. This approach works best for defined length libraries originating from peptides generated by site-specific protease digestion such as trypsin, LysC, GluC, etc. In one example, normalization can be accomplished by denaturing a double-stranded library and allowing the library elements to re-anneal. The abundant library elements re-anneal more quickly than less abundant elements due to the second-order rate constant of bimolecular hybridization kinetics (Bochman, Paeschke et al. 2012). The ssDNA library elements can be separated from the abundant dsDNA library elements using methods known in the art, such as chromatography on hydroxyapatite columns (VanderNoot, et al., 2012, Biotechniques 53:373-380) or treatment of the library with a duplex-specific nuclease (DSN) from Kamchatka crab (Shagin et al., (2002) Genome Res. 12:1935-42) which destroys the dsDNA library elements.

Any combination of fractionation, enrichment, and subtraction methods, of the polypeptides before attachment to the support and/or of the resulting extended nucleic acid library can economize sequencing reads and improve measurement of low abundance species.

In some embodiments, a library of nucleic acids (e.g., extended nucleic acids) is concatenated by ligation or end-complementary PCR to create a long DNA molecule comprising multiple different extended recorder tags (Du et al., (2003) BioTechniques 35:66-72; Muecke et al., (2008) Structure 16:837-841; U.S. Pat. No. 5,834,252, each of which is incorporated by reference in its entirety). This embodiment is preferable for nanopore sequencing in which long strands of DNA are analyzed by the nanopore sequencing device.

In some embodiments, direct single molecule analysis is performed on the nucleic acids (e.g., extended nucleic acids) (see, e.g., Harris et al., (2008) Science 320:106-109). The nucleic acids (e.g., extended nucleic acids) can be analysed directly on the support, such as a flow cell or beads that are compatible for loading onto a flow cell surface (optionally microcell patterned), wherein the flow cell or beads can integrate with a single molecule sequencer or a single molecule decoding instrument. For single molecule decoding, hybridization of several rounds of pooled fluorescently-labelled of decoding oligonucleotides (Gunderson et al., (2004) Genome Res. 14:970-7) can be used to ascertain both the identity and order of the secondary tags within the extended nucleic acids (e.g., on the recording tag). In some embodiments, cycle-specific secondary tags as described above may be used (see also, Gunderson et al., (2004) Genome Res. 14:970-7).

Following sequencing of the nucleic acid libraries (e.g., of extended nucleic acids), the resulting sequences can be collapsed by their UMIs if used and then associated to their corresponding polypeptides and aligned to the totality of the proteome. Resulting sequences can also be collapsed by their compartment tags and associated to their corresponding compartmental proteome, which in a particular embodiment contains only a single or a very limited number of protein molecules. Both protein identification and quantification can easily be derived from this digital peptide information.

### III. KITS AND ARTICLES OF MANUFACTURE

Provided herein are kits and articles of manufacture comprising components for preforming a macromolecule analysis assay. In some embodiments, the kit includes a binding agent comprising a coding tag, which comprises identifying information regarding the binding agent; an adaptor molecule comprising a first hybridization sequence substantially complementary to at least a portion of the coding tag, and a secondary tag. In some embodiment, the binding agent is configured to bind a macromolecule associated with a recording tag and reagents for transferring information from the secondary tag from the adaptor molecule to the recording tag are also provided. In some aspects, the kit includes a plurality or set of adaptor molecules. In some aspects, the kit includes a plurality or set of binding agents. In some embodiments, the set of binding agents is configured to be compatible with the set of adaptor molecules.

In some embodiments, the kits further contain other reagents for treating and analyzing the target macromolecules (e.g., proteins, polypeptides, or peptides). The kits and articles of manufacture may include any one or more of the reagents and components used in the methods described in Section I and II. In some embodiments, the kit comprises reagents for preparing samples, such as for preparing macromolecules from a sample and joining to a support. In some embodiments, the kits optionally include instructions for performing the macromolecule analysis assay. In some embodiments, the kits comprise one or more of the following components: binding agent(s), adaptor molecule(s), solid support(s), recording tag(s), reagent(s) for transferring information, sequencing reagent(s), and/or any needed buffer(s), etc.

In one aspect, provided herein are components used to prepare a reaction mixture. In some preferred embodiments, the reaction mixture is a solution. In preferred embodiments, the reaction mixture includes one or more of the following: adaptor molecule(s), binding agent(s) and associated coding tag(s), solid support(s), recording tag(s), reagent(s) for transferring information, sequencing reagent(s), and/or buffer(s).

In another aspect, disclosed herein is a kit for performing a macromolecule analysis assay comprising a library of binding agents, wherein each binding agent comprises or is associated with a coding tag. In some aspects, disclosed herein is a kit for performing a macromolecule analysis assay comprising a library of adaptor molecules, wherein each adaptor molecule comprises a first hybridization sequence and a secondary tag comprising identifying information regarding the binding moiety. In some examples, the binding moiety is capable of binding to one or more N-terminal, internal, or C-terminal amino acids of the target peptide or polypeptide, or capable of binding to the one or more N-terminal, internal, or C-terminal amino acids of a peptide modified by a functionalizing/modification reagent

In some embodiments, the kits and articles of manufacture further comprise a plurality of nucleic acid molecules or oligonucleotides. In some embodiments, the kits include a plurality of barcodes. The barcode(s) may include a compartment barcode, a partition barcode, a sample barcode, a fraction barcode, or any combination thereof. In some cases, the barcode comprises a unique molecule identifier (UMI). In some examples, the barcode comprises a DNA molecule, DNA with pseudo-complementary bases, an RNA molecule, a BNA molecule, an XNA molecule, a LNA molecule, a PNA molecule, a γPNA molecule, a non-nucleic acid sequenceable polymer, e.g., a polysaccharide, a polypeptide, a peptide, or a polyamide, or a combination thereof. In some embodiments, the barcodes are configured to attach the target macromolecules, e.g., the proteins, in the sample or to attach to nucleic components associated with the targets.

In some embodiments, the kit further comprises reagents for treating the macromolecules, e.g., the proteins. Any combination of fractionation, enrichment, and subtraction methods, of the proteins may be performed. For example, the reagent may be used to fragment or digest the proteins. In some cases, the kit comprises reagents and components to fractionate, isolate, subtract, enrich proteins. In some examples, the kits further comprises a protease such as trypsin, LysN, or LysC. In some embodiments, the kit comprises a support for immobilizing the one or more targets and reagents for immobilizing the target on a support.

In some embodiments, the kit also comprises one or more buffers or reaction fluids necessary for performing any of the steps of the macromolecule analysis assay. Buffers including wash buffers, reaction buffers, and binding buffers, elution buffers and the like are known to those or ordinary skill in the arts. In some embodiments, the kits further include buffers and other components to accompany other reagents described herein. The reagents, buffers, and other components may be provided in vials (such as sealed vials), vessels, ampules, bottles, jars, flexible packaging (e.g., sealed Mylar or plastic bags), and the like. Any of the components of the kits may be sterilized and/or sealed.

In some embodiments, the kit includes one or more reagents for nucleic acid sequence analysis. In some examples, the reagent for sequence analysis is for use in sequencing by synthesis, sequencing by ligation, single molecule sequencing, single molecule fluorescent sequencing, sequencing by hybridization, polony sequencing, ion semiconductor sequencing, pyrosequencing, single molecule real-time sequencing, nanopore-based sequencing, or direct imaging of DNA using advanced microscopy, or any combination thereof.

In addition to above-mentioned components, the subject kits may further include instructions for using the components of the kit to practice the subject methods, i.e., instructions for sample preparation, treatment and/or analysis. The kits described herein may also include other materials desirable from a commercial and user standpoint, including other buffers, diluents, filters, syringes, and package inserts with instructions for performing any methods described herein.

Any of the above-mentioned kit components, and any molecule, molecular complex or conjugate, reagent (e.g., chemical or biological reagents), agent, structure (e.g., support, surface, particle, or bead), reaction intermediate, reaction product, binding complex, or any other article of manufacture disclosed and/or used in the exemplary kits and methods, may be provided separately or in any suitable combination in order to form a kit.

### IV. EXEMPLARY EMBODIMENTS

Among the provided embodiments are:

1. A method for analyzing a macromolecule, comprising the steps of:

(a) providing a macromolecule and an associated recording tag joined to a support;

(b) contacting the macromolecule with a binding agent capable of binding to the macromolecule, wherein the binding agent comprises a coding tag with identifying information regarding the binding agent, to allow binding between the macromolecule and the binding agent;

(c) providing an adaptor molecule comprising a first hybridization sequence substantially complementary to at least a portion of the coding tag, and a secondary tag, to allow hybridization between the adaptor molecule (or the first hybridization sequence) and the coding tag (or the portion of the coding tag);

(d) transferring the information of the secondary tag to the recording tag to generate an extended recording tag; and

analyzing the extended recording tag.

2. The method of embodiment 1, wherein step (b) comprises contacting a plurality of macromolecules with a single binding agent or a plurality of binding agents and step (c) comprises providing a single adaptor molecule or a plurality of adaptor molecules.

3. The method of embodiment 2, wherein the plurality of adaptor molecules comprises at least one adaptor molecule capable of hybridizing to at least one coding tag associated with the binding agent.

4. The method of any one of embodiments 1-3, wherein multiple coding tags associated with the binding agent are configured to hybridize to adaptor molecules comprising the same secondary tag.

5. The method of any one of embodiments 1-4, wherein information from the secondary tag is transferred from the adaptor molecule to the recording tag after the coding tag associated with the binding agent hybridizes with the first hybridization sequence on the adaptor molecule.

6. The method of any one of embodiments 1-5, wherein the adaptor molecule further comprises a second hybridization sequence substantially complementary to a portion of the recording tag.

7. The method of embodiment 6, wherein the second hybridization sequence is substantially complementary to a sequence at the 3′ terminus of the recording tag.

8. The method of any one of embodiments 1-7, wherein the adaptor molecule comprises a DNA molecule, an RNA molecule, a PNA molecule, a BNA molecule, an XNA, molecule, an LNA molecule, a γPNA molecule, or a combination thereof.

9. The method of any one of embodiments 6-8, wherein the second hybridization sequence is at the 3′ terminus of the adaptor molecule.

10. The method of any one of embodiments 6-9, wherein the second hybridization sequence on the adaptor molecule is substantially complementary to a region on the recording tag generated from a previous information transfer of the secondary tag from the adaptor molecule to the recording tag.

11. The method of any one of embodiments 6-10, wherein the second hybridization sequence on the adaptor molecule comprises the secondary tag information or portion thereof on a different adaptor molecule.

12. The method of any one of embodiments 6-11, wherein information transfer of the secondary tag from the adaptor molecule to the recording tag occurs if:

the first hybridization sequence on the adaptor molecule hybridizes to the coding tag of a binding agent; and

the second hybridization sequence of the adaptor molecule hybridizes to a portion of the recording tag.

13. The method of any one of embodiments 1-12, wherein information transfer from the secondary tag to the recording tag is mediated by a ligase, e.g., a DNA ligase.

14. The method of any one of embodiments 1-12, wherein information transfer from the secondary tag to the recording tag is mediated by chemical ligation.

15. The method of any one of embodiments 1-12, wherein information transfer from the secondary tag to the recording tag is mediated by a polymerase, e.g., a DNA polymerase.

16. The method of embodiment 15, wherein the adaptor molecule comprises a spacer to stop extension after transfer of information from the secondary tag to the recording tag.

17. The method of embodiment 16, wherein the spacer is 5′ to the secondary tag on the adaptor molecule.

18. The method of any one of embodiments 1-17, wherein the secondary tag comprises a binding cycle specific sequence.

19. The method of any one of embodiments 1-18, wherein the first hybridization sequence is 5′ to the secondary tag in the adaptor molecule.

20. The method of any one of embodiments 13-18, wherein the first hybridization sequence is 3′ to the secondary tag in the adaptor molecule.

21. The method of any one of embodiments 6-20, wherein the adaptor molecule comprises from 5′ to 3′ direction: the first hybridization sequence, the secondary tag, and the second hybridization sequence.

22. The method of any one of embodiments 1-21, wherein the support is a three-dimensional support (e.g., a porous matrix or a bead).

23. The method of embodiment 22, wherein the support comprises a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combination thereof.

24. The method of any one of embodiments 1-23, wherein the macromolecule is a protein.

25. The method of any one of embodiments 1-23, wherein the macromolecule is a peptide.

26. The method of embodiment 25, wherein the peptide is obtained by fragmenting a protein from a biological sample.

27. The method of any one of embodiments 1-26, wherein the recording tag is a DNA molecule, an RNA molecule, a PNA molecule, a BNA molecule, an XNA, molecule, an LNA molecule, a γPNA molecule, or a combination thereof.

28. The method of any one of embodiments 1-27, which comprises repeating a cycle of steps (b), (c), and (d) one or more times prior to analyzing the extended recording tag.

29. The method of any one of embodiments 24-28, wherein the binding agent is configured to bind to a C-terminal amino acid residue of the protein or peptide.

30. The method of any one of embodiments 24-28, wherein the binding agent is configured to bind to an N-terminal amino acid residue of the protein or peptide.

31. The method of any one of embodiments 24-79, further comprising

(e) removing the N-terminal amino acid (NTAA) of the protein or peptide to expose a new NTAA of the protein or peptide.

32. The method of embodiment 31, wherein a cycle of steps (b), (c), (d) and (e) is repeated one or more times prior to analyzing the extended recording tag.

33. The method of any one of embodiments 24-32, further comprising treating the protein or peptide with a reagent for modifying a terminal amino acid of the protein or peptide.

34. The method of embodiment 33, wherein the reagent for modifying a terminal amino acid of a polypeptide comprises a chemical agent or an enzymatic agent.

35. The method of any one of embodiments 1-34, further comprising removing the binding agent.

36. The method of embodiment 35, wherein the binding agent is removed after transferring the information of the secondary tag to the recording tag.

37. The method of any one of embodiments 1-36, further comprising adding a universal priming site to the extended recording tag.

38. The method of any one of embodiments 1-37, wherein one or more extended recording tags are amplified prior to analysis.

39. The method of any one of embodiments 1-38, wherein analyzing the extended recording tag comprises a nucleic acid sequencing method.

40. The method of embodiment 39, wherein the nucleic acid sequencing method is sequencing by synthesis, sequencing by ligation, sequencing by hybridization, polony sequencing, ion semiconductor sequencing, or pyrosequencing.

41. The method of embodiment 39 or embodiment 40, wherein the nucleic acid sequencing method is single molecule real-time sequencing, nanopore-based sequencing, or direct imaging of DNA using advanced microscopy.

42. The method of any one of embodiments 1-41, wherein the binding agent and the coding tag are joined by a linker.

43. The method of any one of embodiments 1-42, wherein the secondary tag comprises a UMI.

44. The method of any one of embodiments 1-43, wherein the adaptor molecule further comprises a universal priming site.

45. The method of any one of embodiments 1-44, wherein the recording tag comprises a universal priming site.

46. The method of any one of embodiments 1-45, wherein the binding agent is a polypeptide or protein.

47. The method of embodiment 46, wherein the binding agent is an aminopeptidase or variant, mutant, or modified protein thereof; an aminoacyl tRNA synthetase or variant, mutant, or modified protein thereof; an anticalin or variant, mutant, or modified protein thereof; a ClpS, ClpS2, or variant, mutant, or modified protein thereof; a UBR box protein or variant, mutant, or modified protein thereof; or a modified small molecule that binds amino acid(s), i.e. vancomycin or a variant, mutant, or modified molecule thereof; or an antibody or binding fragment thereof; or any combination thereof.

48. The method of any one of embodiments 1-47, wherein the binding agent binds to a single amino acid residue, a dipeptide, a tripeptide or a post-translational modification of a polypeptide macromolecule.

49. A kit for information transfer, comprising:
a binding agent comprising a coding tag, which comprises identifying information regarding the binding agent;
an adaptor molecule comprising a first hybridization sequence substantially complementary to at least a portion of the coding tag, and a secondary tag;
wherein the binding agent is configured to bind a macromolecule associated with a recording tag; and
wherein information from the secondary tag is configured for transfer from the adaptor molecule to the recording tag.

50. The kit of embodiment 49, wherein the kit comprises a plurality of binding agents.

51. The kit of embodiment 49 or embodiment 50, wherein the kit comprises a plurality of adaptor molecules.

52. The kit of embodiment 51, wherein the plurality of adaptor molecules comprises at least one adaptor molecule capable of hybridizing to at least one coding tag associated with the binding agent.

53. The kit of any one of embodiments 50-52, wherein more than one binding agent is associated with the same secondary tag.

54. The kit of any one of embodiments 50-52, wherein multiple coding tags associated with the binding agent are configured to hybridize to adaptor molecules comprising the same secondary tag.

55. The kit of any one of embodiments 1-54, wherein the adaptor molecule further comprises a spacer.

56. The kit of any one of embodiments 1-55, wherein the adaptor molecule further comprises a second hybridization sequence complementary to a portion of the recording tag.

57. The kit of embodiment 56, wherein the second hybridization sequence is complementary to a sequence at the 3′ terminus of the recording tag.

58. The kit of any one of embodiments 49-57, wherein the adaptor molecule comprises a DNA molecule, an RNA molecule, a PNA molecule, a BNA molecule, an XNA, molecule, an LNA molecule, a γPNA molecule, or a combination thereof.

59. The kit of any one of embodiments 56-58, wherein the second hybridization sequence is at the 3′ terminus of the adaptor molecule.

60. The kit of any one of embodiments 56-59, wherein the second hybridization sequence on the adaptor molecule is complementary to a region on the recording tag generated from a previous information transfer of the secondary tag from the adaptor molecule to the recording tag.

61. The kit of any one of embodiments 56-59, wherein the second hybridization sequence on the adaptor molecule comprises the secondary tag information or portion thereof on a different adaptor molecule.

62. The kit of any one of embodiments 49-61, wherein information transfer from the secondary tag to the recording tag is mediated by a ligase, e.g., a DNA ligase.

63. The kit of any one of embodiments 49-61, wherein information transfer from the secondary tag to the recording tag is mediated by chemical ligation.

64. The kit of any one of embodiments 49-61, wherein information transfer from the secondary tag to the recording tag is mediated by a polymerase, e.g., a DNA polymerase.

65. The kit of embodiment 64, wherein the adaptor molecule comprises a spacer to stop extension after transfer of information from the secondary tag to the recording tag.

66. The kit of embodiment 65, wherein the spacer is 5′ to the secondary tag on the adaptor molecule.

67. The kit of any one of embodiments 49-66, wherein the adaptor molecule further comprises a binding cycle specific sequence.

68. The kit of any one of embodiments 49-67, wherein the secondary tag is a binding cycle specific sequence.

69. The kit of any one of embodiments 49-63, 67 and 68, wherein the first hybridization sequence is 5′ to the secondary tag in the adaptor molecule.

70. The kit of any one of embodiments 49-61 and 64-68, wherein the first hybridization sequence is 3′ to the secondary tag in the adaptor molecule.

71. The kit of any one of embodiments 49-68, wherein the adaptor molecule comprises from 5′ to 3′ direction: the first hybridization sequence, the secondary tag, and the second hybridization sequence.

72. The kit of any one of embodiments 49-71, further comprising a support for immobilizing the macromolecule and/or the recording tag.

73. The kit of embodiment 72, wherein the support is a three-dimensional support (e.g., a porous matrix or a bead).

74. The kit of embodiment 73, wherein the support comprises a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combination thereof.

75. The kit of any one of embodiments 49-74, wherein the recording tag is a DNA molecule, an RNA molecule, a PNA molecule, a BNA molecule, an XNA, molecule, an LNA molecule, a γPNA molecule, or a combination thereof.

76. The kit of any one of embodiments 49-75, wherein the adaptor molecule comprises a DNA molecule, an RNA molecule, a PNA molecule, a BNA molecule, an XNA, molecule, an LNA molecule, a γPNA molecule, or a combination thereof.

77. The kit of any one of embodiments 49-76, wherein the binding agent and the coding tag are joined by a linker.

78. The kit of any one of embodiments 49-77, wherein the secondary tag comprises a UMI.

79. The kit of any one of embodiments 49-78, wherein the adaptor molecule further comprises a universal priming site.

80. The kit of any one of embodiments 49-79, wherein the recording tag comprises a universal priming site.

81. The kit of any one of embodiments 49-80, wherein the binding agent is a polypeptide or protein.

82. The kit of embodiment 81, wherein the binding agent is an aminopeptidase or variant, mutant, or modified protein thereof; an aminoacyl tRNA synthetase or variant, mutant, or modified protein thereof; an anticalin or variant, mutant, or modified protein thereof; a ClpS, ClpS2, or variant, mutant, or modified protein thereof; a UBR box protein or variant, mutant, or modified protein thereof; or a modified small molecule that binds amino acid(s), i.e. vancomycin or a variant, mutant, or modified molecule thereof; or an antibody or binding fragment thereof; or any combination thereof.

83. The kit of any one of embodiments 49-82, wherein the binding agent binds to a single amino acid residue, a dipeptide, a tripeptide or a post-translational modification of a polypeptide macromolecule.

84. The method of any one of embodiments 1-48, wherein the first hybridization sequence in the adaptor molecule is complementary to a portion of the coding tag.

85. The method of any one of embodiments 1-48, wherein the first hybridization sequence in the adaptor molecule is complementary to the entire coding tag.

86. The kit of any one of embodiments 49-83, wherein the first hybridization sequence in the adaptor molecule is complementary to a portion of the coding tag.

87. The kit of any one of embodiments 49-83, wherein the first hybridization sequence in the adaptor molecule is complementary to the entire coding tag.

### V. EXAMPLES

The following examples are offered to illustrate but not to limit the methods, compositions, and uses provided herein. Certain aspects of the present invention, including, but not limited to, embodiments for information transfer between coding tags and recording tags, methods for attachment of nucleotide-polypeptide chimera to a support, methods of making nucleotide-polypeptide chimera, methods of generating barcodes, methods of generating specific binders recognizing an N-terminal amino acid of a polypeptide, reagents and methods for modifying and/or removing an N-terminal amino acid from a polypeptide were disclosed in US 20190145982 A1, US 20200348308 A1, US 20200348307 A1, WO 2020/223000, the contents of which are incorporated herein by reference in its entirety.

### Example 1: Exemplary Assay Including Information Transfer Via Splint Adaptor Molecule

This example describes an exemplary assay system including information transfer using a splint adaptor molecule containing a first hybridization sequence complementary to a region on the coding tag and a second hybridization sequence complementary to a region on the recording tag.

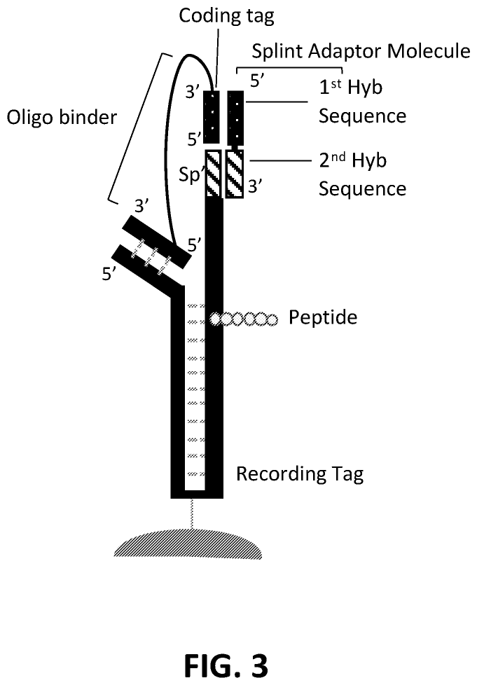

Phosphorylated DNA recording tag was attached to three different peptides, with an amino-terminal sequence of FA, AFA and AA respectively (FA-peptide: FAGVAMPGAEDDVVGSGSGK as set forth in SEQ ID NO: 3; AFA-peptide: AFAGVAMPGAEDDVVGSGSK as set forth in SEQ ID NO: 4; AA-peptide: AAGVAMPGAEDDVVGSGSK as set forth in SEQ ID NO: 5). The DNA recording tag with no peptide attached was also used. The peptide-DNA conjugates and no-peptide DNA recording tags were immobilized on magnetic beads (Dynabeads, Thermo Fisher, USA). For the model assay, an oligo binder that is configured to hybridize to a sequence at the 5′ end of the recording tag was used (FIG. 3). The oligo binder contained a nucleic acid coding tag containing a barcode. Two different coding tag sequences were tested. A DNA splint adaptor molecule was introduced that contained a first hybridization sequence complementary to the coding tag associated with the oligo binder and a second hybridization sequence that is complementary to the spacer region (Sp′) in the recording tags (FIG. 3). To test the effect of mismatched nucleotides in the first hybridization sequence, which is configured to hybridize to the coding tag, or mismatched nucleotides in the second hybridization sequence, which is configured to hybridize to the recording tag, splint adaptor molecules comprising a first hybridization sequence that contained a 3 nucleotide mismatch, a 4 nucleotide mismatch, or a 7 nucleotide (full) mismatch, or a second hybridization sequence that contained a 4 nucleotide mismatch, or a 8 nucleotide (full) mismatch, were used.

The oligo binder and DNA-peptide chimera immobilized beads was incubated with the splint adaptor molecule at 37° C. for 30 minutes, then at room temperature for 15 minutes. After a 1-minute wash in PBST (PBS+ Tween20), the beads were incubated with encoding mixture containing 50 mM Tris-HCl, pH7.5, 2 mM MgSO₄, 50 mM NaCl, 1 mM DTT, 0.1% Tween 20, 0.1 mg/mL BSA, 0.125 mM dNTPs, 0.125 units/μL Klenow fragment (3′->5′ exo-) (MCLAB, USA) at 37° C. for 5 minutes. The beads were washed once with PBST with 10% formamide, once with 0.1 M NaOH and once with PBST with 10% formamide. The resulting beads were resuspended in PB ST. The information of splint adaptor molecule (corresponding to the coding tag) was transferred to the recording tag, thereby generating an extended recording tag. The extended recording tag of the assay was subjected to qPCR with corresponding primers. As shown in Table 1, lower Ct values were obtained when the fully matched splint adaptor molecule was used (0 mismatch) indicating transferred information on the recording tag, whereas the splint adaptor molecule containing mismatches in either the 1st hyb sequence or 2nd hyb sequence produced higher Ct values relative to the 0 mismatch splint adaptor molecule. The data indicate that information corresponding to the coding tag was transferred from the splint adaptor molecule to the recording tag when hybridization occurred via a fully matched splint adaptor molecule.

TABLE 1
qPCR Assessment of Encoding (Information Transfer from Splint Adaptor Molecule)

| | | Splint Mismatch in | Splint Mismatch in | |
|---|---|---|---|---|
| | | 1st Hyb Sequence | 2nd Hyb Sequence | |
| Condition | Coding Tag | (# nucleotides) | (# nucleotides) | Ct Value |
| +Splint Adaptor | Coding Tag 1 | 0 | 0 | 23.4 |
| Molecule | | 3 | 0 | 29.7 |
| | | 4 | 0 | 29.9 |
| | | 7 (all) | 0 | 30.3 |
| | | 0 | 4 | 31.0 |
| | | 0 | 4 | 30.6 |
| | | 0 | 8 (all) | 30.9 |
| | Coding Tag 2 | 0 | 0 | 22.6 |
| | | 3 | 0 | 29.9 |
| | | 4 | 0 | 29.8 |
| | | 7 (all) | 0 | 29.7 |
| No Splint Adaptor | Coding Tag 1 | N/A | N/A | 31.1 |
| Molecule | Coding Tag 2 | N/A | N/A | 29.7 |
| No Klenow | Coding Tag 1 | 0 | 0 | 30.9 |
| | Coding Tag 2 | 0 | 0 | 29.9 |

For next-generation sequencing (NGS), the recording tags were capped to add a universal priming sequence. Capping was done using an extension reaction. The extended recording tags of the assay were subjected to PCR amplification and analyzed by NGS. As shown in Table 2, information transfer from the splint adaptor molecule to the recording tag was observed when the splint adaptor contained 0 mismatched nucleotides with the coding tag. In comparison, low signal was observed when mismatches were present. This was also the case when the splint adaptor molecule was not provided (negative control) or when the Klenow polymerase was not provided (negative control). The data indicates that hybridization between the sequence on the splint adaptor molecule and corresponding complementary region on the coding tag and the recording tag was sufficient to facilitate transfer of information from the splint adaptor molecule, resulting in an extended recording tag.

TABLE 2
NGS Assessment of Encoding (Information
Transfer from Splint Adaptor Molecule)

| | Splint Mismatch | | |
|---|---|---|---|
| | 1st Hyb Sequence | | Fraction of RT |
| Condition | (# nucleotides) | Peptide | reads enclosed |
| +Splint Adaptor | 0 | AA-PA | 0.168 |
| Molecule | 0 | AFA-PA | 0.378 |
| | 0 | FA-PA | 0.348 |
| | 0 | No Peptide | 0.336 |
| | 3 | AA-PA | 0.00 |
| | 3 | AFA-PA | 0.01 |
| | 3 | FA-PA | 0.00 |
| | 3 | No Peptide | 0.01 |
| | 4 | AA-PA | 0.00 |
| | 4 | AFA-PA | 0.00 |
| | 4 | FA-PA | 0.01 |
| | 4 | No Peptide | 0.01 |
| | 7 (all) | AA-PA | 0.00 |
| | 7 (all) | AFA-PA | 0.02 |
| | 7 (all) | FA-PA | 0.00 |
| | 7 (all) | No Peptide | 0.01 |
| No Splint Adaptor | N/A | AA-PA | 0.00 |
| Molecule | N/A | AFA-PA | 0.00 |
| | N/A | FA-PA | 0.01 |
| | N/A | No Peptide | 0.00 |
| No Klenow | 0 | AA-PA | 0.00 |
| | 0 | AFA-PA | 0.00 |
| | 0 | FA-PA | 0.01 |
| | 0 | No Peptide | 0.00 |

### Example 2: Generation of Specific N-Terminal Amino Acid (NTAA) Binders by Phage Display Library Screening

Library construction, phage panning, and clone characterization. High diversity (˜10¹⁰) phage libraries using NNK variant site encoding were constructed targeting residues positions within the pocket of the anticalin. The phage library construction is known in the art and disclosed, for example, in Miersch S, et al., Scalable high throughput selection from phage-displayed synthetic antibody libraries. J Vis Exp. 2015 Jan. 17; (95):51492. doi: 10.3791/51492, which is incorporated herein by reference. Three rounds of selection were used. A pin-based magnetic particle processor (Kingfisher, Thermo) was used for unit-automation of the panning procedure, which enables the handling of 96 magnetic pins, corresponding to the positions of a 96-well microtitre plate, essentially as described in Zoltán Konthur et al., Semi-automated Magnetic Bead-Based Antibody Selection from Phage Display Libraries, Springer Protocols Handbook, Antibody Engineering, pp 267-287, 2010, DOI 10.1007/978-3-642-01144-3_18, which is incorporated herein by reference. Using the disclosed standard protocols, phage libraries were panned against different NTAA target peptides. Clones from the panning output were isolated and characterized using a panel of peptides in a multiplex Luminex binding assay. Specific binders were isolated against a variety of modified and non-modified NTAAs.

In one example, using phage display technology, a highly-selective engineered ClpS2 variant for a NTAA F binder (binder specifically recognizing F at the N-terminus of a polypeptide) was obtained. Agrobacterium tumefaciens ClpS2 (4YJM, starting scaffold MSDSPVDLKPKPKVKPKLERPKLYKVMLLNDDYTPREFVTVVLKAVFRMSEDTGRRV MMTAHRFGSAVVVVCERDIAETKAKEATDLGKEAGFPLMFTTEPEE as set forth in SEQ ID NO: 6) was cloned into a phage display vector with PelB leader sequence at N-terminus. From a subset of positions (L28, N30, D31, D32, Y33, T34, P35, R36, V39, M58, A61, H62, L95), we created different combinations of mutations using NNK degenerate codons (N=A/C/G/T, K=G/T). FA-PA peptide (SEQ ID NO: 3) with biotin was coated onto Streptavidin magnetic beads, and served as the target to pull down binders to FA-PA. AA-PA peptide (SEQ ID NO: 5) is used as a competitor to enrich for specific binders.

In another example, lipocalins were used as starting scaffolds for directed evolution toward modified NTAAs by phage display technology. Anticalins have an intrinsic cup-like binding pocket, highly stable structure, good recombinant expression in E. coli., binding pocket evolvability using phage display, and demonstrated potential for strong and specific binding to small molecules. Many anticalins have an intrinsic ability to bind a modified-dipeptide residue. Based on computational modeling, we designed N-terminal modifier agents (M) such that when combined with the P1 amino acid (N-terminal residue), the M-P1 moiety occupies the anticalin β-barrel core, with the P1 sidechain oriented closer to the surface of the pocket. This design forces the P2 residue (penultimate residue) of the peptide to be located just outside the pocket or affinity determining region and contribute less energy to binding. As one example, Pyrazole methanimine (PMI) is used as the N-terminal modifier agent. Two selective engineered NTAA binders were obtained based on lipocalin/anticalin scaffolds. 31-F binder specifically recognizing F at the N-terminus of a polypeptide was obtained from the following starting scaffold, SEQ ID NO: 7: QVSVQPNFQQDKFLGRWFSAGLASNSSWLREKKAALSMAKSVVAPATDGGLNLTSTF LRKNQCETRTMLLQPAGSLGSYSYRSPHFGSTYSVSVVETDYDQYALLYSQGSKGPGE DFRMATLYSRTQTPRAELKEKFTAFSKAQGFTEDTIVFLPQTDKCMTEQ. From a subset of positions (19, 22, 29, 33, 38, 41, 53, 55, 57, 66, 68, 81, 86, 90, 92, 105, 107, 117, 119, 121, 123), we created different combinations of mutations using NNK degenerate codons; PMI1-FXGG-peg9-K(biotin) peptide (SEQ ID NO: 8) was coated onto Streptavidin magnetic beads and served as the target to pull down binders that recognizes PMI1-F at the N-terminus of a polypeptide. Also, 44-L binder specifically recognizing L at the N-terminus of a polypeptide was obtained from the following starting scaffold, SEQ ID NO: 9: GPVPTPPDNIQVQENFNISRIYGKWYNLAIGSTSPWLKKIMDRMTVSTLVLGEGATEAEI SMTSTRWRKGVCEETSGAYEKTDTDGKFLYHKSKWNITMESYVVHTNYDEYAIFLTK KFSRHHGPTITAKLYGRAPQLRETLLQDFRVVAQGVGIPEDSIFTMADRGECVPGEQ.

From a subset of positions (27, 30, 36, 37, 40, 43, 46, 48, 62, 64, 66, 75, 79, 99, 101, 114, 116, 128, 130, 132), we created different combinations of mutations using NNK degenerate codons; PMI1-LXGG-peg9-K(biotin) peptide (SEQ ID NO: 10) was coated onto Streptavidin magnetic beads and served as the target to pull down binders that recognizes PMI1-L at the N-terminus of a polypeptide.

### Example 3: Exemplary Assay Showing Information Transfer Via an Adaptor Molecule

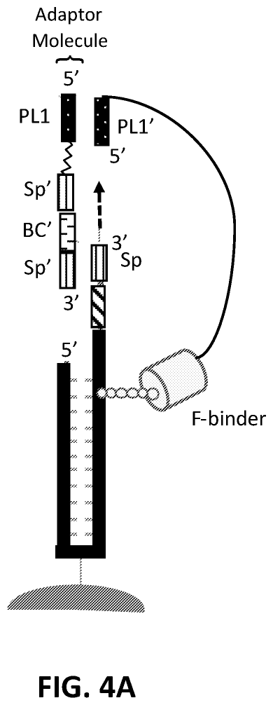

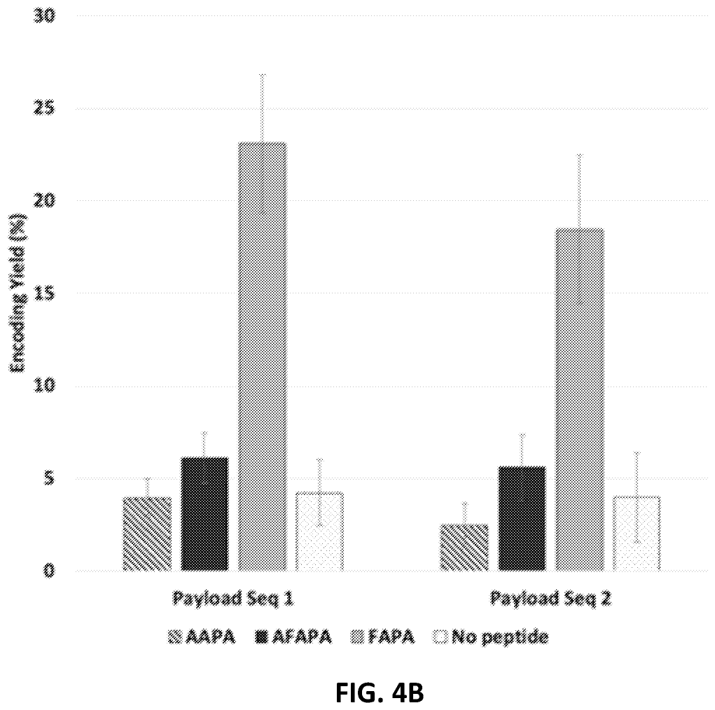

This example describes a specific embodiment for information transfer between a peptide molecule conjugated to a recording tag and a DNA-conjugated binder molecule conjugated that recognizes an N-terminal amino acid of the peptide molecule (FIG. 4). Information transfer, in this embodiment, occurs via a splint adaptor molecule containing a first hybridization sequence (PL1′) complementary to a region on the coding tag (PL1), followed by a PEG-based linker, a spacer sequence (Sp′), a barcode sequence (BC′) and another spacer sequence (Sp′) complementary to a region on the recording tag (Sp) (FIG. 4A).

In a particular example, a set of four recording tag (RT)-peptide chimeras were created by first “activating” the 5′ amine on the RT oligonucleotides by coupling to TCO-PEG12-NHS ester (Click Chemistry Tools). After TCO activation, the RT oligonucleotide, designed with an internal alkyne group, was coupled to azide-containing FA, AA and AF peptides (FA-peptide or FA-PA: FAGVAMPGAEDDVVGSGSGK as set forth in SEQ ID NO: 3; AFA-peptide or AFA-PA: AFAGVAMPGAEDDVVGSGSK as set forth in SEQ ID NO: 4; AA-peptide or AA-PA: AAGVAMPGAEDDVVGSGSK as set forth in SEQ ID NO: 5). These peptides having N-terminal FA, AA and AF amino acid sequences and an internal PA epitope were individually attached to recording tag oligonucleotides, amRT_Cs2, amRT_Cs4, and amRT_Cs5 (SEQ ID NOs: 11-13), respectively. A fourth recording tag, amRT_Cs1 (SEQ ID NOs: 14), was included as a no peptide control. An F-binder binding agent was conjugated to the coding tag oligonucleotide, amCT_s7 (SEQ ID NO: 15) comprised of the 8-mer barcode. The four chimeras that contain FA peptide, AA peptide, AF peptide or no peptide were combined and immobilized to mTet beads using iEDDA TCO-mTet chemistry. This 4-plex model system has been demonstrated an intra-molecular single molecule binding and specific encoding of an engineered F-binder on low recording tag density beads (1:10,000 and 1:100,000), see US 20200348308 A1. Absolute loading of the four different chimeras on beads was measured by an universal PA antibody since all three peptide types contained a PA antigen sequence, and all four chimeras were loaded in roughly equal amounts on the beads.

To increase efficiency of capture of the four RT-peptide chimeras on beads, a hybridization-based immobilization was employed as disclosed in WO 2020/223000. The four RT-peptide chimeras were hybridized and ligated to hairpin capture DNAs that were chemically immobilized on magnetic beads (Dynabeads, Thermo Fisher, USA). The capture nucleic acids were conjugated to the beads using trans-cyclooctene (TCO) and methyltetrazine (mTet)-based click chemistry. TCO-modified short hairpin capture nucleic acids (16 basepair stem, 5 base loop, 24 base 5′ overhang) were reacted with mTet-coated magnetic beads. Phosphorylated RT-peptide chimeras (10 nM) were annealed to the hairpin DNAs attached to beads in 5×SSC, 0.02% SDS, and incubated for 30 minutes at 37° C. The beads were washed once with PBST and resuspended in 1× Quick ligation solution (New England Biolabs, USA) with T4 DNA ligase. After a 30-minute incubation at 25° C., the beads were washed three times with 0.1 M NaOH+0.1% Tween 20 and three times with PBST. The total immobilized RT-peptide chimeras were quantified by qPCR using specific primer sets. Alternatively, peptides can be immobilized onto beads using a non-hybridization based method that did not involve a ligation step. The non-hybridization based method can be performed by incubating 30 μM TCO-modified DNA-tagged peptides including amino FA-terminal peptides, amino AFA-terminal peptides, and amino AA-terminal peptides, with mTet-coated magnetic beads overnight at 25° C. as described in US 20200348308 A1.

After capturing the four RT-peptide chimeras on beads, a binding and encoding assay was performed utilizing splint adaptor molecules as shown in FIG. 4A. A specific F-binder engineered from ClpS2 (obtained by Phage display library screening as disclosed in the previous example) conjugated with two different coding tags—hybridization sequences Payload seq 1 (PL1) and Payload seq 1 (PL2) was used for the assay. PL1 and PL2 were designed as random sequences that contain ˜50% CG and do not interact with corresponding barcode and spacer sequences on the adaptor molecules. The complementary sequences of PL1 and PL2 (am-PL1′ and am-PL2′, SEQ ID NOs: 16-17) were attached to SpyTag via a PEG linker, and the resulting SpyTag-PL1′ and SpyTag-PL2′ were conjugated to F binder-SpyCatcher fusion protein, thus creating F binder-PL1′ and F binder-PL2′ fusions.

For the encoding assay, which comprises an information transfer between the coding tag and the recording tag, the F binder-PL1′ and F binder-PL2′ were mixed with splint adaptor molecules, CT_PL1 S6 and CT_PL2 S6 (SEQ ID NOs: 18-19), respectively, in 1:4 molar ratio, forming two binder mixes. Both adaptor molecules also include the C3 spacer at the 3′ terminus of the oligonucleotide. The C3 spacer is a three carbon spacer. The C3 spacer located at the 3′ terminus of the oligonucleotide prohibits extension of the oligonucleotide by a polymerase and prevents ligation at the 3′ end. The architecture of splint adaptor molecules as shown in FIG. 4A allows for hybridization between splint adaptor molecule, coding tag and recording tag, providing conditions for transfer information between the coding tag and the recording tag. The bead-immobilized RT-peptide chimeras were incubated with 200 nM of each binder mix in 150 μL of PB ST (PBS+ Tween20) at room temperature. After 30 minutes incubation, the beads were washed twice with 200 μL of PBST with 500 mM NaCl and resuspended in encoding mixture containing 50 mM Tris-HCl, pH7.5, 2 mM MgSO₄, 50 mM NaCl, 1 mM DTT, 0.1% Tween 20, 0.1 mg/mL BSA, 0.125 mM dNTPs, 0.125 units/μL Klenow fragment (3′->5′ exo-) (MCLAB, USA) at 37° C. for 5 minutes. The beads were washed once with 0.1 M NaOH+0.01% Tween 20 and twice with PB ST. The information of splint adaptor molecule (corresponding to the coding tag) was transferred to the recording tag, thereby generating an extended recording tag. After all the encoding cycles are completed, 0.4 mM of a nucleic acid (the capping oligonucleotide set forth in SEQ ID NO: 44) was added into the encoding mixture and incubated at 25° C. for 10 minutes to add a universal priming sequence to the recording tags (extended or unextended) using an extension reaction to generate a final product for next-generation sequencing (NGS) readout. The extended recording tags of the assay were subjected to PCR amplification and analyzed by NGS. Encoding yield on each peptide was obtained by evaluating percentage of recording tags containing adaptor molecule's barcode information that was transferred during the assay. For both PL1 and PL2 sequences used, a high encoding yield during the assay was observed only for the target peptide FA-PA having F as the N-terminal amino acid (FIG. 4B), showing specificity of the information transfer for the cognate F-binder fusions. In an alternate embodiment, the splint adapter molecule can be annealed to the binder-peptide complex post binding and washing.

### Example 4: Exemplary Multi-Cycle Assay Showing Information Transfer Via an Adaptor Molecule

An exemplary two-cycle encoding assay is performed as follows. The first cycle encoding is performed as described in Example 3. Chimeric molecules comprised of peptide-DNA recording tags are immobilized on magnetic beads as described in Example 3. A mixture of several N-terminal modified selective binders is used in the assay to interact with an immobilized polypeptide; each binder comprises an engineered protein specifically recognizing a particular N-terminal amino acid of the polypeptide and associated with a coding tag containing information about the binder. The coding tag specific for each binder (PL′) is attached to SpyTag via a PEG linker, and the resulting SpyTag-PL′ is conjugated to binder-SpyCatcher fusion protein.

A set of 20 exemplar coding tag barcode sequences (PL′ sequences) of 20 nucleotides in length include the following sequences (SEQ ID NOs: 20-39) based on Elmas, A., et al. (2013). “Designing DNA Barcodes Orthogonal in Melting Temperature by Simulated Annealing Optimization.” Nucleic acid therapeutics 23: 140-151):

(SEQ ID NO: 20)
TGGTAGAGCCACAAACAGCC,
(SEQ ID NO: 21)
GGTACAAGCAACGATCTCCA,
(SEQ ID NO: 22)
GGACCATCTGAATCATGCGC,
(SEQ ID NO: 23)
GGATGACACGAACTCACGAC,
(SEQ ID NO: 24)
GGCGATCACAGACATTAACC,
(SEQ ID NO: 25)
CACAGCCGATAATTGCAGAC,
(SEQ ID NO: 26)
GGTACAGACACTGCGACAAC,
(SEQ ID NO: 27)
GTGGCAATTCGTCGCAATAC,
(SEQ ID NO: 28)
GGGTCATCACGGCTCATCAT,
(SEQ ID NO: 29)
GCCAGATGTCAACACAGCTA,
(SEQ ID NO: 30)
CCGCCAAACAAATGTGTGCA,
(SEQ ID NO: 31)
ATACACGCTCGGAAGACTGC,
(SEQ ID NO: 32)
ATGATGACCGCACTGACTGG,
(SEQ ID NO: 33)
GGACAGCAGATCCACCTAAG,
(SEQ ID NO: 34)
CCTGTGAGAGAAGCAGACAC,
(SEQ ID NO: 35)
CCGACAGATCAAGGCAGTTA,
(SEQ ID NO: 36)
AATCGCAGCCAAGTGAGTGA,
(SEQ ID NO: 37)
ATAGATGACGCACCACGGTC,
(SEQ ID NO: 38)
AGACACGACACACTGGCTTA,
(SEQ ID NO: 39)
AGGAGACGCCACATCGTATC.

For each binder, the binder-PL′ conjugates are mixed with corresponding adaptor molecules, containing a complementary PL sequences, a spacer and a unique barcode (BC′), in 1:4 molar ratio. The binder-specific coding tag adapter sequences are comprised of 15 to 30-mer barcodes which have been designed to hybrid efficiently and orthogonally to other members barcodes. Exemplar approaches to generating orthogonal hybridizing sets of barcodes (20-25 nt.) are disclosed in: Elmas, A., et al. (2013). “Designing DNA Barcodes Orthogonal in Melting Temperature by Simulated Annealing Optimization.” Nucleic acid therapeutics 23: 140-151; Pierce, S. E., et al. (2006). “A unique and universal molecular barcode array.” Nat Methods 3(8): 601-603; Cook, M. A., et al. (2008). “Systematic validation and atomic force microscopy of non-covalent short oligonucleotide barcode microarrays.” PLoS One 3(2): e1546; Xu, Q., et al. (2009). “Design of 240,000 orthogonal 25mer DNA barcode probes.” Proc Natl Acad Sci USA 106(7): 2289-2294; Casini, A., et al. (2014). “R2oDNA designer: computational design of biologically neutral synthetic DNA sequences.” ACS Synth Biol 3(8): 525-528.

In addition to good orthogonal hybridization properties amongst the members of the set, the barcodes can be further filtered to remove cross-reactivity with other DNA components of the ProteoCode system including universal priming sequences, spacer sequences, recording tag barcodes, etc.

In the case of two binders A and B used in the assay, conjugated to coding tag sequences, PLA1′ (TGGTAGAGCCACAAACAGCC, SEQ ID NO: 20) and PLB2′ (GGTACAAGCAACGATCTCCA, SEQ ID NO: 21), respectively, beads with polypeptide-recording tag fusion molecules are incubated at 30° C. temperature with 150 μL of mixture containing 200 nM binder A-PLA1′+ adaptor A_1^{st} cycle (GGCTGTTTGTGGCTCTACCA---GGTAAGAGCGACTGTAGTGTG, SEQ ID NO: 40; where “---” is a C3 spacer) and 200 nM binder B-PLB2′+ adaptor B_1st cycle (TGGAGATCGTTGCTTGTACC---GGTAAGAGCCGATGTAGTGTG, SEQ ID NO: 41) in PBS-T (PBS+ Tween 20). Both adaptor molecules (adaptor A_1^{st} cycle and adaptor B_1^{st} cycle) also include the C3 spacer at the 3′ terminus of the oligonucleotide. The C3 spacer is a three carbon spacer. The C3 spacer located at the 3′ terminus of the oligonucleotide prohibits extension of the oligonucleotide by a polymerase and prevents ligation at the 3′ end.

After 30 minutes incubation, the beads are washed twice with 200 μL PBS-T with 500 mM NaCl, and resuspended in encoding mixture containing 50 mM Tris-HCl, pH7.5, 2 mM MgSO4, 50 mM NaCl, 1 mM DTT, 0.1% Tween 20, 0.1 mg/mL BSA, 0.125 mM dNTPs, 0.125 units/μL Klenow fragment (3′-5′ exo-) (MCLAB, USA) at 37° C. for 5 minutes. The beads are washed once with 0.1 M NaOH+0.01% Tween 20 and twice with PBS-T. If during the first cycle specific binding occurs between the NTAA of the polypeptide and the binder, the information regarding this binder is transferred from binding-specific barcode in the adaptor molecule to the recording tag (becomes encoded in the recording tag). In addition, the adaptor molecules may also contain a cycle-specific barcode (in this case a unique barcode for the first cycle) that will be also encoded in the recording tag after information transfer. After information transfer, the N-terminal amino acid of the polypeptide is cleaved off by mild Edman-like methods described, for example in US 20200348307 A1 or WO 2020223133 A1. Alternatively, the N-terminal amino acid of the polypeptide is cleaved off enzymatically by engineered cleavases described, for example in WO 2020198264 A1. The cleavage exposes a new N-terminal amino acid of the polypeptide, and the system is ready for the second cycle. During the second cycle of encoding the same set of binder coding tag combinations can be used (for example, binders A and B), but with a new set of adaptor molecules. For example, a mixture will contain 200 nM binder A-PL1′+adaptor molecule1_2^{nd} cycle (GGCTGTTTGTGGCTCTACCA---AGAGATGGCACGTGGTAAGAG, SEQ ID NO: 42) and 200 nM binder B-PL2′+adaptor molecule2_2^{nd} cycle (TGGAGATCGTTGCTTGTACC---AGAGATGGTGCGTGGTAAGAG, SEQ ID NO: 43) in PBS-T. Both adaptor molecules (adaptor molecule1_2^{nd} cycle and adaptor molecule2_2^{nd} cycle) also include the C3 spacer at the 3′ terminus of the oligonucleotide. Adaptor molecules used for the second cycle will contain a cycle-specific barcode unique for the second cycle. The encoding conditions are the same for the first and the second cycle. After the second cycle encoding, either the third cycle can be done (following the NTAA cleavage) with new adaptor molecules containing a cycle-specific barcode unique for the third cycle, or the extended recording tag is capped by addition of a universal priming sequence to finish the encoding process. The capped recording tag is subjected to PCR amplification followed by sequencing and analysis using a next-generation sequencing method. The described approach overcomes a necessity to make and use a new pool of binder fusions for each cycle of encoding; rather, a new pool of cycle-specific adapter molecules is used for each cycle together with a single universal set of coding tag-conjugated binders.

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

TABLE 3
SEQUENCE TABLE

| SEQ | | |
|---|---|---|
| ID NO | Sequence (5′-3′) | Description |
| 1 | AATGATACGGCGACCACCGA | P5 primer |
| 2 | CAAGCAGAAGACGGCATACGAGAT | P7 primer |
| 3 | FAGVAMPGAEDDVVGSGSGK | FA-PA |
| 4 | AFAGVAMPGAEDDVVGSGSK | AFA-PA |
| 5 | AAGVAMPGAEDDVVGSGSK | AA-PA |
| 6 | MSDSPVDLKPKPKVKPKLERPKLYKVMLLN | scaffold for binder |
| | DDYTPREFVTVVLKAVFRMSEDTGRRVMM | selection 1 |
| | TAHRFGSAVVVVCERDIAETKAKEATDLGK | |
| | EAGFPLMFTTEPEE | |
| 7 | QVSVQPNFQQDKFLGRWFSAGLASNSSWLR | scaffold for binder |
| | EKKAALSMAKSVVAPATDGGLNLTSTFLRK | selection 2 |
| | NQCETRTMLLQPAGSLGSYSYRSPHFGSTYS | |
| | VSVVETDYDQYALLYSQGSKGPGEDFRMAT | |
| | LYSRTQTPRAELKEKFTAFSKAQGFTEDTIVF | |
| | LPQTDKCMTEQ | |
| 8 | PMI1-FXGG-peg9-K(biotin) | bait peptide for binder |
| | | selection 1 |
| 9 | GPVPTPPDNIQVQENFNISRIYGKWYNLAIGS | scaffold for binder |
| | TSPWLKKIMDRMTVSTLVLGEGATEAEISMT | selection 3 |
| | STRWRKGVCEETSGAYEKTDTDGKFLYHKS | |
| | KWNITMESYVVHTNYDEYAIFLTKKFSRHH | |
| | GPTITAKLYGRAPQLRETLLQDFRVVAQGVG | |
| | IPEDSIFTMADRGECVPGEQ | |
| 10 | PMI1-LXGG-peg9-K(biotin) | bait peptide for binder |
| | | selection 2 |
| 11 | ttcgtagtcc gcgacactag nnnnnnnnnn gttaatggac | amRT_Cs2 |
| | tgagtg | oligonucleotide, n is a, |
| | | c, g, or t |
| 12 | ttcgtagtcc gcgacactag nnnnnnnnnn cagtaccgac | amRT_Cs4 |
| | tgagtg | oligonucleotide, n is a, |
| | | c, g, or t |
| 13 | ttcgtagtcc gcgacactag nnnnnnnnnn gttggttaac | amRT_Cs5 |
| | tgagtg | oligonucleotide, n is a, |
| | | c, g, or t |
| 14 | ttcgtagtcc gcgacactag nnnnnnnnnn ttaagtcgac | amRT_Cs1 |
| | tgagtg | oligonucleotide, n is a, |
| | | c, g, or t |
| 15 | cactcagtca gactattcac tcagt | coding tag |
| | | oligonucleotide |
| 16 | CATAGACTAGTAGCCGGAAC | am-PL1′ |
| | | oligonucleotide |
| 17 | TCGACGTAATGACACCGCTC | am- |
| | | PL2′ oligonucleotide |
| 18 | GTTCCGGCTACTAGTCTATG-peg6- | CT_PL1_S6 adaptor |
| | CACTCAGTTTTTCCTGTCACTCAGT | molecule |
| 19 | GAGCGGTGTCATTACGTCGA-peg6- | CT_PL2_S6 adaptor |
| | CACTCAGTTTTTCCTGTCACTCAGT | molecule |
| 20 | TGGTAGAGCCACAAACAGCC | coding tag barcode |
| | | sequence 1 |
| 21 | GGTACAAGCAACGATCTCCA | coding tag barcode |
| | | sequence 2 |
| 22 | GGACCATCTGAATCATGCGC | coding tag barcode |
| | | sequence 3 |
| 23 | GGATGACACGAACTCACGAC | coding tag barcode |
| | | sequence 4 |
| 24 | GGCGATCACAGACATTAACC | coding tag barcode |
| | | sequence 5 |
| 25 | CACAGCCGATAATTGCAGAC | coding tag barcode |
| | | sequence 6 |
| 26 | GGTACAGACACTGCGACAAC | coding tag barcode |
| | | sequence 7 |
| 27 | GTGGCAATTCGTCGCAATAC | coding tag barcode |
| | | sequence 8 |
| 28 | GGGTCATCACGGCTCATCAT | coding tag barcode |
| | | sequence 9 |
| 29 | GCCAGATGTCAACACAGCTA | coding tag barcode |
| | | sequence 10 |
| 30 | CCGCCAAACAAATGTGTGCA | coding tag barcode |
| | | sequence 11 |
| 31 | ATACACGCTCGGAAGACTGC | coding tag barcode |
| | | sequence 12 |
| 32 | ATGATGACCGCACTGACTGG | coding tag barcode |
| | | sequence 13 |
| 33 | GGACAGCAGATCCACCTAAG | coding tag barcode |
| | | sequence 14 |
| 34 | CCTGTGAGAGAAGCAGACAC | coding tag barcode |
| | | sequence 15 |
| 35 | CCGACAGATCAAGGCAGTTA | coding tag barcode |
| | | sequence 16 |
| 36 | AATCGCAGCCAAGTGAGTGA | coding tag barcode |
| | | sequence 17 |
| 37 | ATAGATGACGCACCACGGTC | coding tag barcode |
| | | sequence 18 |
| 38 | AGACACGACACACTGGCTTA | coding tag barcode |
| | | sequence 19 |
| 39 | AGGAGACGCCACATCGTATC | coding tag barcode |
| | | sequence 20 |
| 40 | GGCTGTTTGTGGCTCTACCA-c3 spacer- | adaptor molecule 1 |
| | GGTAAGAGCGACTGTAGTGTG/3SpC3/ | |
| 41 | TGGAGATCGTTGCTTGTACC-c3 spacer- | adaptor molecule 2 |
| | GGTAAGAGCCGATGTAGTGTG/3SpC3/ | |
| 42 | GGCTGTTTGTGGCTCTACCA-c3 spacer- | adaptor molecule 3 |
| | AGAGATGGCACGTGGTAAGAG/3SpC3/ | |
| 43 | TGGAGATCGTTGCTTGTACC-c3 spacer- | adaptor molecule 4 |
| | AGAGATGGTGCGTGGTAAGAG/3SpC3/ | |
| 44 | GAC TGG AGT TCA GAC GTG TGC TCT TCC | Capping |
| | GAT CTT TCT ACT CTT CTC ACT CAG | oligonucleotide |
| | T/3SpC3/ | |

PMI1=Pyrazole methanimine modification at the N-terminus

peg9 spacer=internal nine atom polyethylene glycol spacer

peg6 spacer=internal six atom polyethylene glycol spacer

-c3 spacer-=an internal three-carbon spacer

/3 SpC3/=3′-terminal three-carbon spacer

1. A method for analyzing a macromolecule, comprising the steps of:
(a) providing a macromolecule and an associated recording tag joined to a support;
(b) contacting the macromolecule with a binding agent capable of binding to the macromolecule, wherein the binding agent comprises a coding tag with identifying information regarding the binding agent, to allow binding between the macromolecule and the binding agent;
(c) providing an adaptor molecule comprising a first hybridization sequence and a secondary tag, wherein the first hybridization sequence is substantially complementary to at least a portion of the coding tag, to allow hybridization between the first hybridization sequence and the coding tag, wherein step (c) is performed before, after or simultaneously with step (b);
(d) transferring information of the secondary tag to the recording tag to generate an extended recording tag, wherein the information of the secondary tag is transferred from the adaptor molecule to the recording tag after the coding tag associated with the binding agent hybridizes with the first hybridization sequence on the adaptor molecule; and
(e) analyzing the extended recording tag.

2. The method of claim 1, wherein step (b) comprises contacting a plurality of macromolecules with a plurality of binding agents and step (c) comprises providing a plurality of adaptor molecules, wherein the plurality of adaptor molecules comprises at least one adaptor molecule capable of hybridizing to at least one coding tag associated with the binding agent.

3. The method of claim 1, wherein multiple coding tags associated with the binding agent are configured to hybridize to adaptor molecules comprising the same secondary tag.

4. The method of claim 1, wherein the adaptor molecule further comprises a second hybridization sequence substantially complementary to a sequence at the 3′ terminus of the recording tag or substantially complementary to a region on the recording tag generated from a previous information transfer of the secondary tag from the adaptor molecule to the recording tag, and wherein information transfer of the secondary tag from the adaptor molecule to the recording tag occurs after:
the first hybridization sequence on the adaptor molecule hybridizes to the coding tag of the binding agent; and
the second hybridization sequence of the adaptor molecule hybridizes to a portion of the recording tag.

5. The method of claim 1, wherein the macromolecule is a polypeptide, analyzing the macromolecule comprises determining at least a portion of an amino acid sequence of the polypeptide, and recording tag, the coding tag and the adaptor molecule comprise a DNA molecule, an RNA molecule, a PNA molecule, a BNA molecule, an XNA, molecule, an LNA molecule, a γPNA molecule, or a combination thereof.

6. The method of claim 4, wherein the second hybridization sequence on the adaptor molecule comprises the secondary tag information or portion thereof on a different adaptor molecule.

7. The method of claim 1, wherein information transfer from the secondary tag to the recording tag is mediated by a DNA ligase or DNA polymerase.

8. (canceled)

9. The method of claim 7, wherein the adaptor molecule comprises a spacer to stop extension after transfer of information from the secondary tag to the recording tag.

10. The method of claim 1, wherein the secondary tag comprises a binding cycle specific sequence.

11. The method of claim 10, wherein the adaptor molecule comprises from 5′ to 3′ direction: the first hybridization sequence, the secondary tag, and the second hybridization sequence, and the secondary tag comprises an identifying information regarding the binding agent, a binding cycle-specific barcode, a unique molecular identifier, or a combination thereof.

12. The method of claim 5, wherein the binding agent is configured to bind to an N-terminal amino acid (NTAA) residue of the polypeptide.

13. The method of claim 5, wherein the method further comprises the following step: (a′) modifying an N-terminal amino acid (NTAA) residue of the polypeptide, thereby producing a modified NTAA residue, and the binding agent is configured to bind to the modified NTAA residue of the polypeptide.

14. The method of claim 13, further comprising the following steps after step (d) and before step (e): (i) removing the modified NTAA residue of the polypeptide to expose immediately adjacent amino acid residue of the polypeptide as a new NTAA residue; (ii) modifying the new NTAA residue, thereby producing a new modified NTAA residue; (iii) contacting the polypeptide with a second binding agent capable of binding to the new modified NTAA residue of the polypeptide, wherein the second binding agent comprises a second coding tag with identifying information regarding the second binding agent, to allow binding between the polypeptide and the binding agent; (iv) providing a second adaptor molecule comprising a second first hybridization sequence substantially complementary to at least a portion of the second coding tag, and a second secondary tag, to allow hybridization between the second first hybridization sequence and the second coding tag; (v) transferring an information of the second secondary tag to the extended recording tag, wherein the information of the second secondary tag is transferred from the second adaptor molecule to the extended recording tag after the second coding tag associated with the second binding agent hybridizes with the second first hybridization sequence on the second adaptor molecule.

15-16. (canceled)

17. A kit for analyzing a macromolecule, comprising:
a binding agent comprising a coding tag, which comprises identifying information regarding the binding agent, wherein the binding agent is configured to bind to the macromolecule associated with a recording tag joined to a support;
an adaptor molecule comprising a first hybridization sequence substantially complementary to at least a portion of the coding tag, and a secondary tag,
wherein an information of the secondary tag is configured for transferring from the adaptor molecule to the recording tag to generate an extended recording tag after the coding tag associated with the binding agent hybridizes with the first hybridization sequence on the adaptor molecule;
optionally, a means for transferring the information of the secondary tag or a means for analyzing the extended recording tag.

18. The kit of claim 17, wherein the kit comprises a plurality of binding agents or a plurality of adaptor molecules, wherein the plurality of adaptor molecules comprises at least one adaptor molecule capable of hybridizing to at least one coding tag associated with the binding agent.

19. The kit of claim 17, wherein the adaptor molecule further comprises a second hybridization sequence substantially complementary to a sequence at the 3′ terminus of the recording tag or substantially complementary to a region on the recording tag generated from a previous information transfer of the secondary tag from the adaptor molecule to the recording tag, and wherein information transfer of the secondary tag from the adaptor molecule to the recording tag is configured to occur after:
the first hybridization sequence on the adaptor molecule hybridizes to the coding tag of the binding agent; and
the second hybridization sequence of the adaptor molecule hybridizes to a portion of the recording tag.

20. The kit of claim 17, wherein the macromolecule is a polypeptide, analyzing the macromolecule comprises determining at least a portion of an amino acid sequence of the polypeptide, and recording tag, the coding tag and the adaptor molecule comprise a DNA molecule, an RNA molecule, a PNA molecule, a BNA molecule, an XNA, molecule, an LNA molecule, a γPNA molecule, or a combination thereof.

21. The kit of claim 20, wherein the means for transfer the information of the secondary tag comprises DNA ligase or DNA polymerase, and the means for analyzing the extended recording tag comprises a sequencing primer.

22. The kit of claim 19, wherein the adaptor molecule comprises from 5′ to 3′ direction: the first hybridization sequence, the secondary tag, and the second hybridization sequence, and the secondary tag comprises an identifying information regarding the binding agent, a binding cycle-specific barcode, a unique molecular identifier, or a combination thereof.

23. The kit of claim 20, further comprising a support for immobilizing the polypeptide and/or the recording tag.

24. (canceled)
